SEQUENCE LISTING (4 sequences)

SEQ ID NO: 1 - DNA, 1426 bp, Artificial Sequence
  DNA sequence encoding the alpha-RANKL-1 antibody heavy chain
aagcttgaccaccatggagtttgggctgagctggctttttcttgtggctattttaaaaggtgtccagtgtgaggtgcagctgttggagtctgggggaggcttggtacagcctggggggtccctgagactctcctgtgcagcctctggattcacctttagcagctatgccatgagctgggtccgccaggctccagggaaggggctggagtgggtctcaggtattactgggagtggtggtagtacatactacgcagactccgtgaagggccggttcaccatctccagagacaattccaagaacacgctgtatctgcaaatgaacagcctgagagccgaggacacggccgtatattactgtgcgaaagatccagggactacggtgattatgagttggttcgacccctggggccagggaaccctggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataagtcgac

SEQ ID NO: 2 - protein, 467 aa, Artificial Sequence
  Amino acid sequence of the alpha-RANKL-1 antibody heavy chain
MEFGLSWLFLVAILKGVQCEVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 3 - DNA, 728 bp, Artificial Sequence
  DNA sequence encoding the alpha-RANKL-1 antibody kappa light chain
tctagaccaccatggaaaccccagcgcagcttctcttcctcctgctactctggctcccagataccaccggagaaattgtgttgacgcagtctccaggcaccctgtctttgtctccaggggaaagagccaccctctcctgtagggccagtcagagtgttcgcggcaggtacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgttttactgtcagcagtatggtagttcacctcggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgataagtcgac

SEQ ID NO: 4 - protein, 235 aa, Artificial Sequence
  Amino acid sequence of the alpha-RANKL-1 antibody kappa light chain
METPAQLLFLLLLWLPDTTGEIVLTQSPGTLSLSPGERATLSCRASQSVRGRYLAWYQQKPGQAPRLLIYGASSRATGIPDRFSGSGSGTDFTLTISRLEPEDFAVFYCQQYGSSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2011/0060290 A1
(43) Pub. Date: Mar. 10, 2011

(54) STABILIZED PROTEIN COMPOSITIONS

(75) Inventors: Roberta Bonk, Newbury Park, CA (US); Mingda Eu, Oak Park, CA (US); Amy Huinker, Santa Monica, CA (US); Monica Pallitto, Newbury Park, CA (US); Margaret Ricci, CaMarillo, CA (US); Nicole Stackhouse, Bothell, WA (US)

(73) Assignee: Amgen Inc., Thousand Oaks, CA (US)

(21) Appl. No.: 12/866,400

(22) PCT Filed: Feb. 5, 2009
(86) PCT No.: PCT/US09/00759

§ 371 Date: Nov. 8, 2010

Related U.S. Application Data
(60) Provisional application No. 61/065,065, filed on Feb. 7, 2008.

(51) Int. Cl. A61M 5/00; A61K 39/395; A61P 19/08; A61P 29/00

(52) U.S. Cl. 604/181; 424/158.1; 424/133.1; 424/145.1

(57) ABSTRACT

Stabilized compositions of specific binding agents to RANKL, specific binding agents to TNF, and/or specific binding agents to IL-1R1 in containers are provided. Methods of making and using such compositions are also provided.

This application claims the benefit of U.S. Provisional Application No. 61/065,065, filed Feb. 7, 2008. U.S. Provisional Application No. 61/065,065 is incorporated herein by reference in its entirety for any purpose.

### FIELD

Stabilized compositions of specific binding agents to RANKL, specific binding agents to TNF, and/or specific binding agents to IL-1R1 in containers are provided. Methods of making and using such compositions are also provided.

### BACKGROUND

Certain therapeutic compositions comprise specific binding agents. In certain instances, therapeutic compositions are placed in containers, for example, for storage and shipping. In certain instances, such containers are compatible with storage and shipping conditions, as well as the mode of administration, for example, including, but not limited to, subcutaneous, intramuscular or intravenous injection. Certain exemplary containers include, but are not limited to, an ampoule, disposable syringe, including, but not limited to, disposable syringe suitable for prefilling, and multiple dose vial made of glass or plastic. In certain instances, a therapeutic composition is contained in a prefilled syringe, for example, a syringe into which a manufacturer has placed the therapeutic composition.

Therapeutic compositions in containers can, in certain instances, form particles and/or show aggregation upon exposure to shipping and/or storage conditions. Such compositions which exhibit particle formation and/or aggregation, in certain instances, are not suitable for administration and must be disposed of. It is desirable, in certain instances, to provide stabilized therapeutic compositions in containers which, when exposed to shipping and/or storage conditions, are less susceptible to particle formation and/or aggregation.

### SUMMARY

In certain embodiments, a prefilled syringe containing a composition comprising a specific binding agent is provided, wherein the specific binding agent contained in the prefilled syringe is stabilized.

In certain embodiments, a prefilled syringe containing a composition comprising a specific binding agent is provided, wherein a headspace between the composition and a syringe closure is minimized, and wherein the specific binding agent contained in the prefilled syringe is stabilized.

A method of preparing a prefilled syringe comprising introducing into the syringe a composition comprising a specific binding agent such that a headspace between the composition and a syringe closure is minimized, and wherein the specific binding agent contained in the prefilled syringe is stabilized.

In certain embodiments, a method for stabilizing a specific binding agent in a composition is provided, wherein the composition is contained in a prefilled syringe, comprising placing the composition in the prefilled syringe such that a headspace between the composition and a syringe closure is minimized, and wherein the specific binding agent contained in the prefilled syringe is stabilized.

### BRIEF DESCRIPTION OF THE FIGURES

FIG. 1 shows the stability of αRANKL-1 compositions at various protein concentrations incubated in vials at 4° C. for 24 months, and analyzed at various time points by native SEC-HPLC, according to the work discussed in Example 1. (A) Percent main peak (monomer); (B) percent aggregate (pre-peak).

FIG. 2 shows the stability under static conditions of αRANKL-1 compositions at various protein concentrations, after incubating in prefilled glass luer lock syringes or prefilled glass staked needle syringes at 4° C. for 24 weeks, and analyzed at various time points by native SEC-HPLC, according to the work discussed in Example 1.

FIG. 3 shows the percent main peak (monomer) of αRANKL-1 compositions in a polysorbate-free formulation in COP plastic (Resin CZ®) prefilled syringes after incubation at 4° C. for 4 weeks, 10 weeks, 22 weeks, 32 weeks, or 52 weeks, under static conditions or after shipping, and analyzed at various times by native SEC-HPLC, according to the work discussed in Example 1.

FIG. 4 shows the size distribution of sTNFR:Fc samples. The figure shows the sub-visible particle size, as indicated by the intensity weighted size distribution, of sTNFR:Fc samples subjected to various prefilling and shipping conditions according to the work discussed in Example 2.

FIG. 5 (A) is a schematic drawing of a staked-needle syringe and syringe components; FIG. 5 (B) is a schematic drawing of a prefilled syringe showing a headspace that is not minimized.

FIG. 6 shows a prefilled syringe containing a composition comprising αRANKL-1 and a headspace, or a minimized headspace, according to the work discussed in Example 2; (A) headspace not minimized, showing a headspace of 4.5 mm; (B) minimized headspace showing (left side): a minimized headspace of 1.5 mm with a meniscus; and (right side): a minimized headspace with a visible air bubble.

FIG. 7 shows a cDNA sequence encoding the αRANKL-1 antibody heavy chain (SEQ ID NO: 1). The figure shows the DNA sequence of the heavy chain expression plasmid beginning at a HindIII site, through a SalI site. The start codon begins at nucleotide 14, and the stop codon begins at nucleotide 1415.

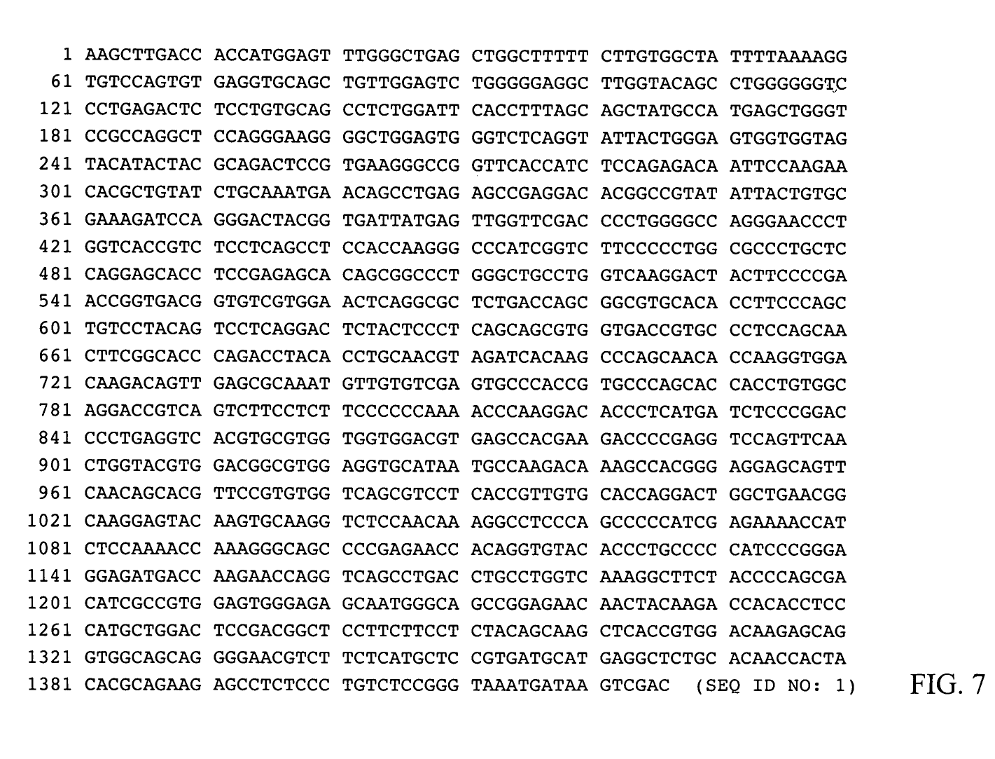

FIG. 8 shows the amino acid sequence of the αRANKL-1 antibody heavy chain (SEQ ID NO: 2). The IgG2 signal peptide is underlined, the variable region is in capital letters and is not underlined, and the constant region is in lower case.

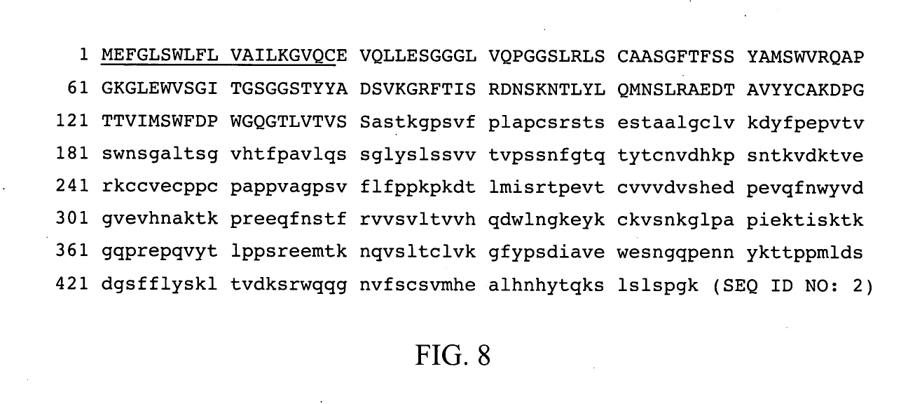

FIG. 9 shows a cDNA sequence encoding the αRANKL-1 antibody kappa light chain (SEQ ID NO: 3). The figure shows the DNA sequence of the kappa chain expression plasmid sequence from an XbaI site through a SalI site. The start codon begins at nucleotide 12; and the stop codon begins at nucleotide 717.

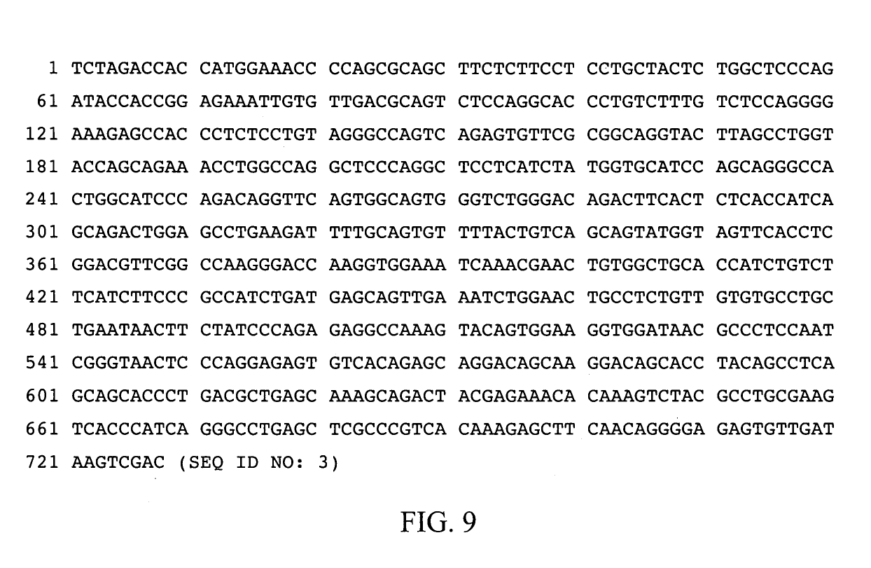

FIG. 10 shows the amino acid sequence of the αRANKL-1 antibody kappa light chain (SEQ ID NO: 4). The kappa signal peptide is underlined, the variable region is in capital letters and not underlined, and the constant region is in lower case.

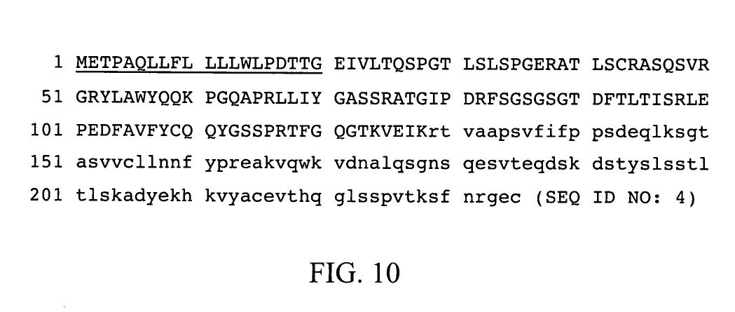

### DETAILED DESCRIPTION OF CERTAIN EMBODIMENTS

The section headings used herein are for organizational purposes only and are not to be construed as limiting the subject matter described. All documents or portions of documents cited in this application, including but not limited to patents, patent applications, articles, books, and treatises, are expressly incorporated by reference herein in their entirety for any purpose. In the event that one or more of the documents, or portions of documents, incorporated by reference defines a term that contradicts that term's definition in this application, this application controls.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)). Unless specific definitions are provided, the nomenclatures utilized in connection with, and the laboratory procedures and techniques of, analytical chemistry, synthetic organic chemistry, and medicinal and pharmaceutical chemistry described herein are those well known and commonly used in the art. Standard techniques may be used for chemical syntheses, chemical analyses, pharmaceutical preparation, formulation, delivery, and treatment of patients.

In this application, the use of the singular includes the plural unless specifically stated otherwise. In this application, the word “a” or “an” means “at least one” unless specifically stated otherwise. In this application, the use of “or” means “and/or” unless specifically stated otherwise. In the context of a multiple dependent claim, the use of “or” refers back to more than one preceding independent or dependent claim in the alternative only. Furthermore, the use of the term “including,” as well as other forms, such as “includes” and “included,” is not limiting. Also, terms such as “element” or “component” encompass both elements or components comprising one unit and elements or components that comprise more than one unit unless specifically stated otherwise.

Receptor Activator of NF-κB Ligand (RANKL), also known as Osteoprotegerin Ligand (OPGL), a member of the tumor necrosis factor (TNF) family of cytokines, promotes formation of osteoclasts through binding to the receptor, RANK. In certain instances, increased osteoclast activity correlates with a number of osteopenic disorders, including, but not limited to, post-menopausal osteoporosis, Paget's disease, lytic bone metastases, and rheumatoid arthritis. Therefore, a reduction in RANKL activity may result in a decrease in osteoclast activity and may reduce the severity of osteopenic disorders. Certain specific binding agents to RANKL, including, but not limited to, antibodies, have been described. See, e.g., U.S. Publication No. 2004/0033535, published Feb. 19, 2004, which is hereby incorporated by reference for any purpose.

Interleukin-1 (IL-1) is a cytokine associated with the inflammatory response. In certain instances, IL-1 stimulates cellular responses by interacting with a heterodimeric receptor complex comprised of two transmembrane proteins, IL-1 receptor type I (IL-1R1) and IL-1 receptor accessory protein (IL-1RAcP). It has been reported that IL-1 first binds to IL-1R1; IL-1RAcP is then recruited to this complex (Greenfeder et al., 1995, J. Biol. Chem. 270:13757-13765; Yoon and Dinarello, 1998, J. Immunology 160:3170-3179; Cullinan et al., 1998, J. Immunology 161:5614-5620), followed by signal transduction resulting in the induction of a cellular response. It has been postulated that, in certain instances, preventing IL-1 signaling by inhibiting IL-1 from binding IL-1 receptor, for example, IL-1R1, may be useful therapeutically for treating certain IL-1 mediated diseases. In certain instances, specific binding agents to IL-1R1 inhibit IL-1 binding to IL-1 receptor. Certain specific binding agents to IL-1R1, including, but not limited to, antibodies, have been described. See, e.g., U.S. Publication No. 2004/0097712, published May 20, 2004, which is hereby incorporated by reference for any purpose.

Tumor necrosis factor-α (TNFα, also known as cachectin) and tumor necrosis factor-β (TNFβ, also known as lymphotoxin) are homologous mammalian endogenous secretory proteins capable of inducing a wide variety of effects on a large number of cell types. The significant similarities in the structural and functional characteristics of these two cytokines have resulted in their collective description as “TNF.” Complementary cDNA clones encoding TNFα (Pennica et al., Nature 312:724, 1984) and TNFβ (Gray et al., Nature 312:721, 1984) have been isolated, permitting further structural and biological characterization of TNF.

TNF proteins initiate their biological effect on cells, in certain instances, by binding to specific TNF receptor (TNF-R) proteins expressed on the plasma membrane of a TNF-responsive cell. In addition to cell surface receptors for TNF, soluble proteins from human urine capable of binding TNF have also been identified (Peetre et al., Eur. J. Haematol. 41:414, 1988; Seckinger et al., J. Exp. Med. 167:1511, 1988; Seckinger et al., J. Biol. Chem. 264:11966, 1989; UK Patent Application, Publ. No. 2 218 101 A to Seckinger et al.; Engelmann et al., J. Biol. Chem. 264:11974, 1989). In addition, certain specific binding agents to TNF, and certain specific binding agents to TNF-R, including, but not limited to, polypeptides, soluble polypeptides, including, but not limited to, soluble fusion polypeptides, and antibodies, have been described. See, e.g., Mohler et al., J. Immunol. 151:1548-1561, 1993; U.S. Pat. No. 5,945,397, which are hereby incorporated by reference for any purpose.

### CERTAIN DEFINITIONS

As utilized in accordance with the present disclosure, the following terms, unless otherwise indicated, shall be understood to have the following meanings.

The term “receptor activator of NF-κB ligand” or “RANKL” refers to a polypeptide which promotes formation of osteoclasts through binding to a receptor activator of NF-κB (“RANK”). RANKL is also called “osteoprotegerin ligand” or “OPGL.” The term “RANKL” includes fragments of RANKL, as well as related polypeptides, which include, but are not limited to, allelic variants, splice variants, derivative variants, substitution variants, deletion variants, and/or insertion variants, fusion polypeptides, and interspecies homologs. In certain embodiments, a RANKL polypeptide includes terminal residues, such as, but not limited to, leader sequence residues, targeting residues, amino terminal methionine residues, lysine residues, tag residues and/or fusion protein residues.

The term “interleukin-1 receptor type 1” or “IL-1R1” refers to a polypeptide which is a transmembrane receptor that is stimulated by an inflammatory cytokine known as interleukin-1 (“IL-1”). The term “IL-1R1” includes fragments of IL-1R1, as well as related polypeptides, which include, but are not limited to, allelic variants, splice variants, derivative variants, substitution variants, deletion variants, and/or insertion variants, fusion polypeptides, and interspecies homologs. In certain embodiments, an IL-1R1 polypeptide includes terminal residues, such as, but not limited to, leader sequence residues, targeting residues, amino terminal methionine residues, lysine residues, tag residues and/or fusion protein residues.

The term “TNF receptor” or “TNF-R” refers to a polypeptide having an amino acid sequence of a native mammalian TNF receptor, or fragments thereof, as well as related polypeptides, which include, but are not limited to, allelic variants, splice variants, derivative variants, substitution variants, deletion variants, and/or insertion variants, fusion polypeptides, and interspecies homologs. Certain exemplary methods and assays to determine biological activity of TNF-R have been described, e.g., in U.S. Pat. No. 5,945,397; and Mohler et al., J. Immunol. 151:1548-1561 (1993). In certain embodiments, a TNF receptor includes terminal residues, such as, but not limited to, leader sequence residues, targeting residues, amino terminal methionine residues, lysine residues, tag residues and/or fusion protein residues. TNF-R is capable of binding TNF ligand. In certain embodiments, TNF-R transduces a biological signal initiated by a TNF ligand binding to a cell. In certain embodiments, TNF-R is capable of binding anti-TNF-R antibodies raised against TNF-R from natural (i.e., nonrecombinant) sources. The mature full-length human TNF-R is a glycoprotein having a molecular weight of about 80 kilodaltons (kDa). The terms “TNF receptor” or “TNF-R” include, but are not limited to, variants or subunits of native polypeptides having at least 20 amino acids and which exhibit at least some biological activity in common with TNF-R, for example, soluble TNF-R constructs which are devoid of a transmembrane region (and are secreted from the cell) but retain the ability to bind TNF. Various exemplary TNF-Rs, including soluble TNF-Rs, are disclosed, for example, in U.S. Pat. No. 5,945,397 and Mohler et al., J. Immunol. 151:1548-1561 (1993). Native human TNF-R is disclosed, for example, in U.S. Pat. No. 5,945,397, Smith et al., Science 248:1019-1023 (1990), Loetscher et al., Cell 61: 351-359 (1990), and Schall et al., Cell 61: 361-370 (1990).

The term “soluble TNF-R” or “sTNF-R” refers to a polypeptide having an amino acid sequence corresponding to all or part of the extracellular region of a native TNF-R, for example, including but not limited to, huTNF-RΔ235, huTNF-RΔ185 and huTNF-RΔ163, or a variant of amino acids 1-163, amino acids 1-185, or amino acids 1-235 of native human TNF-R as described in Smith et al., Science 248:1019-1023 (1990), and which are biologically active in that they bind to TNF ligand. In certain embodiments, sTNF-R is etanercept. Etanercept is a recombinant fusion protein that contains the extracellular domain of the p75 sTNF-R attached to a Fc fragment of a human IgG antibody. The amino acid sequence of etanercept was published in Clinical Pharmacology and Therapeutics 66(2):209, 1999, incorporated herein by reference, and the protein is available for sale under the tradename Enbrel® (Amgen Inc.).

The nomenclature for TNF-R and sTNF-R follows the convention of naming the protein (e.g., TNF-R) preceded by either hu (for human) or mu (for murine) and followed by a Δ (to designate a deletion) and the number of the C-terminal amino acid. For example, huTNF-RΔ235 refers to human TNF-R having Asp²³⁵ as the C-terminal amino acid (i.e., a polypeptide having the sequence of amino acids 1-235 of native human TNF-R as described in Smith et al., Science 248:1019-1023 (1990)). In the absence of any human or murine species designation, TNF-R refers generically to mammalian TNF-R. Similarly, in the absence of any specific designation for deletion mutants, the term TNF-R means all forms of TNF-R, including variants which possess TNF-R biological activity. Certain exemplary TNF-Rs include polypeptides which vary from the sequences described above by one or more substitutions, deletions, or additions, and which retain the ability to bind TNF or inhibit TNF signal transduction activity via cell surface bound TNF receptor proteins, for example huTNF-RΔx, wherein x is selected from any one of amino acids 163-235 of native human TNF-R as described in Smith et al., Science 248:1019-1023 (1990). In certain embodiments, analogous deletions are made to murine TNF-R (“muTNF-R”). In certain embodiments, inhibition of TNF signal transduction activity is determined by transfecting cells with recombinant TNF-R DNAs to obtain recombinant receptor expression. In such embodiments, the transfected cells are then contacted with TNF and the resulting metabolic effects examined. If an effect results which is attributable to the action of the ligand, then the recombinant receptor has signal transduction activity. Certain exemplary procedures for determining whether a polypeptide has signal transduction activity are disclosed, e.g., in Idzerda et al., J. Exp. Med. 171:861 (1990); Curtis et al., Proc. Natl. Acad. Sci. USA 86:3045 (1989); Prywes et al., EMBO J. 5:2179 (1986) and Chou et al., J. Biol. Chem. 262:1842 (1987). Alternatively, in certain embodiments, primary cells or cell lines which express an endogenous TNF receptor and have a detectable biological response to TNF are utilized.

The term “comprising,” when used in connection with an amino acid sequence, means that a compound may include additional amino acids on either or both of the N- or C-termini of the given sequence.

In the context of polypeptides, two or more polypeptides are “operably linked” if each linked polypeptide is able to function in its intended manner. A polypeptide that is able to function in its intended manner when operably linked to another polypeptide may or may not be able to function in its intended manner when not operably linked to another polypeptide. For example, in certain embodiments, a first polypeptide may be unable to function in its intended manner when unlinked, but may be stabilized by being linked to a second polypeptide such that it becomes able to function in its intended manner. Alternatively, in certain embodiments, a first polypeptide may be able to function in its intended manner when unlinked, and may retain that ability when operably linked to a second polypeptide.

As used herein, two or more polypeptides are “fused” when the two or more polypeptides are linked by translating them as a single contiguous polypeptide sequence or by synthesizing them as a single contiguous polypeptide sequence. In certain embodiments, two or more fused polypeptides may have been translated in vivo from two or more operably linked polynucleotide coding sequences. In certain embodiments, two or more fused polypeptides may have been translated in vitro from two or more operably linked polynucleotide coding sequences.

As used herein, two or more polypeptides are “operably fused” if each linked polypeptide is able to function in its intended manner.

In certain embodiments, a first polypeptide that contains two or more distinct polypeptide units is considered to be linked to a second polypeptide so long as at least one of the distinct polypeptide units of the first polypeptide is linked to the second polypeptide.

In certain embodiments, the language “a first polypeptide linked to a second polypeptide” encompasses situations where: (a) only one molecule of a first polypeptide is linked to only one molecule of a second polypeptide; (b) only one molecule of a first polypeptide is linked to more than one molecule of a second polypeptide; (c) more than one molecule of a first polypeptide is linked to only one molecule of a second polypeptide; and (d) more than one molecule of a first polypeptide is linked to more than one molecule of a second polypeptide. In certain embodiments, when a linked molecule comprises more than one molecule of a first polypeptide and only one molecule of a second polypeptide, all or fewer than all of the molecules of the first polypeptide may be covalently or noncovalently linked to the second polypeptide. In certain embodiments, when a linked molecule comprises more than one molecule of a first polypeptide, one or more molecules of the first polypeptide may be covalently or noncovalently linked to other molecules of the first polypeptide.

As used herein, a “flexible linker” refers to any linker that is not predicted by one skilled in the art, according to its chemical structure, to be fixed in three-dimensional space. In certain embodiments, a peptide linker comprising three or more amino acids is a flexible linker.

In the context of polypeptides, two or more polypeptides are “attached” if a first polypeptide is fused, operably fused, linked, and/or operably linked to one or more polypeptides.

The term “specific binding agent” refers to a natural or non-natural molecule that specifically binds to a target. Examples of specific binding agents include, but are not limited to, proteins, peptides, nucleic acids, carbohydrates, lipids; and small molecule compounds. In certain embodiments, a specific binding agent is an immunoglobulin. In certain embodiments, a specific binding agent is an immunoglobulin fragment. In certain embodiments, a specific binding agent is an antibody. In certain embodiments, a specific binding agent is an antigen binding region.

The term “specific binding agent to RANKL” refers to a specific binding agent that specifically binds any portion of RANKL. In certain embodiments, a specific binding agent to RANKL is an immunoglobulin. In certain embodiments, a specific binding agent to RANKL is an immunoglobulin fragment. In certain embodiments, a specific binding agent to RANKL is an antibody to RANKL. In certain embodiments, a specific binding agent is an antigen binding region.

The term “specific binding agent to IL-1R1” refers to a specific binding agent that specifically binds any portion of IL-1R1. In certain embodiments, a specific binding agent to IL-1R1 is an immunoglobulin. In certain embodiments, a specific binding agent to IL-1R1 is an immunoglobulin fragment. In certain embodiments, a specific binding agent to IL-1R1 is an antibody to IL-1R1. In certain embodiments, a specific binding agent is an antigen binding region.

The term “specific binding agent to TNF” refers to a specific binding agent that specifically binds any portion of TNF. In certain embodiments, a specific binding agent to TNF is a polypeptide. In certain embodiments, a specific binding agent to TNF is a soluble polypeptide. In certain embodiments, a specific binding agent to TNF is a soluble polypeptide operably fused to a second polypeptide, wherein the second polypeptide is not a specific binding agent to TNF. Such second polypeptides include for example, but are not limited to, Fc and Fc fragment. In certain embodiments, a specific binding agent to TNF is an immunoglobulin. In certain embodiments, a specific binding agent to TNF is an immunoglobulin fragment. In certain embodiments, a specific binding agent to TNF is an antibody to TNF. In certain embodiments, a specific binding agent is an antigen binding region.

The term “specific binding agent to TNF-R” refers to a specific binding agent that specifically binds any portion of TNF-R. In certain embodiments, a specific binding agent to TNF-R is an immunoglobulin. In certain embodiments, a specific binding agent to TNF-R is an immunoglobulin fragment. In certain embodiments, a specific binding agent to TNF-R is an antibody to TNF-R. In certain embodiments, a specific binding agent is an antigen binding region.

The term “specifically binds” refers to the ability of a specific binding agent to bind to a target with greater affinity than it binds to a non-target. In certain embodiments, specific binding refers to binding for a target with an affinity that is at least 10, 50, 100, 250, 500, or 1000 times greater than the affinity for a non-target. In certain embodiments, affinity is determined by an affinity ELISA assay. In certain embodiments, affinity is determined by a BIAcore® assay. In certain embodiments, affinity is determined by a kinetic method. In certain embodiments, affinity is determined by an equilibrium/solution method.

The term “target” refers to a molecule or a portion of a molecule capable of being bound by a specific binding agent. In certain embodiments, a target may have one or more epitopes. In certain embodiments, a target is an antigen.

The term “epitope” refers to a portion of a molecule capable of being bound by a specific binding agent. Exemplary epitopes may comprise any polypeptide determinant capable of specific binding to an immunoglobulin and/or T-cell receptor. Exemplary epitope determinants include, but are not limited to, chemically active surface groupings of molecules, for example, but not limited to, amino acids, sugar side chains, phosphoryl groups, and sulfonyl groups. In certain embodiments, epitope determinants may have specific three dimensional structural characteristics, and/or specific charge characteristics. In certain embodiments, an epitope is a region of an antigen that is bound by an antibody. Epitopes may be contiguous or non-contiguous. In certain embodiments, epitopes may be mimetic in that they comprise a three dimensional structure that is similar to an epitope used to generate the antibody, yet comprise none or only some of the amino acid residues found in that epitope used to generate the antibody.

“Antibody” or “antibody peptide(s)” both refer to an intact antibody, or a fragment thereof. In certain embodiments, the fragment includes contiguous portions of an intact antibody. In certain embodiments, the fragment includes non-contiguous portions of an intact antibody. In certain embodiments, the antibody fragment may be a binding fragment that competes with the intact antibody for specific binding. The term “antibody” also encompasses polyclonal antibodies and monoclonal antibodies. In certain embodiments, binding fragments are produced by recombinant DNA techniques. In certain embodiments, binding fragments are produced by enzymatic or chemical cleavage of intact antibodies. Binding fragments include, but are not limited to, Fab, Fab′, F(ab′)2, Fv, scFv, maxibodies, and single-chain antibodies. Non-antigen binding fragments include, but are not limited to, Fc fragments.

The term “polyclonal antibody” refers to a heterogeneous mixture of antibodies that bind to different epitopes of the same antigen.

The term “monoclonal antibodies” refers to a collection of antibodies encoded by the same nucleic acid molecule. In certain embodiments, monoclonal antibodies are produced by a single hybridoma or other cell line, or by a transgenic mammal. Monoclonal antibodies typically recognize the same epitope. The term “monoclonal” is not limited to any particular method for making an antibody.

“Chimeric antibody” refers to an antibody that has an antibody variable region of a first species fused to another molecule, for example, an antibody constant region of another second species. See, e.g., U.S. Pat. No. 4,816,567 and Morrison et al., Proc Natl Acad Sci (USA), 81:6851-6855 (1985). In certain embodiments, the first species may be different from the second species. In certain embodiments, the first species may be the same as the second species. In certain embodiments, a chimeric antibody is a CDR-grafted antibody.

The term “CDR-grafted antibody” refers to an antibody in which the CDR from one antibody is inserted into the framework of another antibody. In certain embodiments, the antibody from which the CDR is derived and the antibody from which the framework is derived are of different species. In certain embodiments, the antibody from which the CDR is derived and the antibody from which the framework is derived are of different isotypes.

The term “multi-specific antibody” refers to an antibody wherein two or more variable regions bind to different epitopes. The epitopes may be on the same or different targets. In certain embodiments, a multi-specific antibody is a “bi-specific antibody,” which recognizes two different epitopes on the same or different antigens.

The term “catalytic antibody” refers to an antibody in which one or more catalytic moieties is attached. In certain embodiments, a catalytic antibody is a cytotoxic antibody, which comprise a cytotoxic moiety.

The term “humanized antibody” refers to an antibody in which all or part of an antibody framework region is derived from a human, but all or part of one or more CDR regions is derived from another species, for example, including, but not limited to, a mouse.

The term “fully human antibody” refers to an antibody in which both the CDR and the framework comprise substantially human sequences. In certain embodiments, fully human antibodies are produced in non-human mammals, including, but not limited to, mice, rats, and lagomorphs. In certain embodiments, fully human antibodies are produced in hybridoma cells. In certain embodiments, fully human antibodies are produced recombinantly.

The term “heavy chain” includes any polypeptide having sufficient variable region sequence to confer specificity for a target. A full-length heavy chain includes a variable region domain, V_{H}, and three constant region domains, C_{H}1, C_{H}2, and C_{H}3. The V_{H} domain is at the amino-terminus of the polypeptide, and the C_{H}3 domain is at the carboxy-terminus. The term “heavy chain”, as used herein, encompasses a full-length heavy chain and fragments thereof.

The term “light chain” includes any polypeptide having sufficient variable region sequence to confer specificity for a target. A full-length light chain includes a variable region domain, V_{L}, and a constant region domain, C_{L}. Like the heavy chain, the variable region domain of the light chain is at the amino-terminus of the polypeptide. The term “light chain”, as used herein, encompasses a full-length light chain and fragments thereof.

The term “Fab fragment” refers to an antibody comprising one light chain and the C_{H}1 and variable regions of one heavy chain. The heavy chain of a Fab fragment cannot form a disulfide bond with another heavy chain. In certain embodiments, the heavy chain of a Fab fragment forms a disulfide bond with the light chain of a Fab fragment.

The term “Fab′ fragment” refers to an antibody comprising one light chain, the variable and C_{H}1 regions of one heavy chain, and some of the constant region between the C_{H}1 and C_{H}2 domains of the heavy chain. In certain embodiments, an interchain disulfide bond can be formed between two heavy chains of an Fab′ fragment to form a F(ab′)₂ molecule.

The term “F(ab′)₂ molecule” refers to an antibody comprising two Fab′ fragments connected by an interchain disulfide bond formed between two heavy chains.

An “Fv molecule” comprises the variable regions from both the heavy and light chains, but lacks the constant regions. A single chain variable fragment (scFv) comprises variable regions from both a heavy and a light chain wherein the heavy and light chain variable regions are fused to form a single polypeptide chain which forms an antigen-binding region. In certain embodiments, a scFV comprises a single polypeptide chain. A single-chain antibody comprises a scFV. In certain embodiments, a single-chain antibody comprises one or more additional polypeptides fused to a scFv. Exemplary additional polypeptides include, but are not limited to, one or more constant regions. Exemplary single-chain antibodies are discussed, e.g., in WO 88/01649 and U.S. Pat. Nos. 4,946,778 and 5,260,203.

The term “maxibody” refers to a scFv fused (may be by a linker or direct attachment) to an Fc or an Fc fragment. In certain embodiments, a single chain antibody is a maxibody. In certain embodiments, a single chain antibody is a maxibody that binds to HGF. Exemplary Ig-like domain-Fc fusions are disclosed in U.S. Pat. No. 6,117,655.

An “Fc fragment” comprises the C_{H}2 and C_{H}3 domains of the heavy chain and contains some of the constant region, between the C_{H}1 and C_{H}2 domains, such that an interchain disulfide bond can be formed between two heavy chains.

The terms “variable region” and “variable domain” are used interchangeably herein to refer to a portion of the light and/or heavy chains of an antibody. In various instances, variable domains include approximately the amino-terminal 120 to 130 amino acids in the heavy chain and about 100 to 110 amino-terminal amino acids in the light chain. In certain embodiments, variable regions of different antibodies differ extensively in amino acid sequence even among antibodies of the same species. The variable region of an antibody, in various instances, determines specificity of a particular antibody for its target.

The term “antigen binding fragment” refers to a polypeptide fragment comprising at least the variable domain of an immunoglobulin heavy chain and at least the variable domain of an immunoglobulin light chain. In certain embodiments, an antigen binding fragment is capable of binding to a ligand, preventing binding of the ligand to its receptor, and thereby interrupting a biological response resulting from ligand binding to the receptor. In certain embodiments, an antigen binding fragment is capable of binding to a receptor, preventing binding of the ligand to its receptor, and thereby interrupting a biological response resulting from ligand binding to the receptor. In certain embodiments, an antigen binding fragment is capable of binding a receptor and activating that receptor. In certain embodiments, an antigen binding fragment is capable of binding a receptor and inactivating that receptor.

The term “naturally-occurring” as applied to an object refers to the fact that an object can be found in nature. For example, a polypeptide or polynucleotide sequence that is present in an organism (including viruses) that can be isolated from a source in nature and which has not been intentionally modified by man in the laboratory or otherwise is naturally-occurring.

The term “isolated polynucleotide” refers to a polynucleotide of genomic, cDNA, or synthetic origin or some combination thereof, which by virtue of its origin the “isolated polynucleotide” (1) is not associated with all or a portion of a polynucleotide in which the “isolated polynucleotide” is found in nature, (2) is linked to a polynucleotide which it is not linked to in nature, or (3) does not occur in nature as part of a larger sequence.

The term “operably linked” refers to components that are in a relationship permitting them to function in their intended manner. For example, in the context of a polynucleotide sequence, a control sequence may be “operably linked” to a coding sequence when the control sequence and coding sequence are in association with each other in such a way that expression of the coding sequence is achieved under conditions compatible with the functioning of the control sequence.

The term “control sequence” refers to polynucleotide sequences which may effect the expression and processing of coding sequences with which they are in association. The nature of such control sequences may differ depending upon the host organism. Certain exemplary control sequences for prokaryotes include, but are not limited to, promoters, ribosomal binding sites, and transcription termination sequences. Certain exemplary control sequences for eukaryotes include, but are not limited to, promoters, enhancers, and transcription termination sequences. In certain embodiments, “control sequences” can include leader sequences and/or fusion partner sequences.

The terms “isolated polypeptide” and “isolated peptide” refer to any polypeptide that (1) is free of at least some proteins with which it would normally be found, (2) is essentially free of other proteins from the same source, e.g., from the same species, (3) is expressed by a cell from a different species, or (4) does not occur in nature.

The terms “polypeptide,” “peptide,” and “protein” are used interchangeably herein and refer to a polymer of two or more amino acids joined to each other by peptide bonds or modified peptide bonds, i.e., peptide isosteres. The terms apply to amino acid polymers containing naturally occurring amino acids as well as amino acid polymers in which one or more amino acid residues is a non-naturally occurring amino acid or a chemical analogue of a naturally occurring amino acid. An amino acid polymer may contain one or more amino acid residues that has been modified by one or more natural processes, such as post-translational processing, and/or one or more amino acid residues that has been modified by one or more chemical modification techniques known in the art.

As used herein, the twenty conventional amino acids and their abbreviations follow conventional usage. See Immunology—A Synthesis (2nd Edition, E. S. Golub and D. R. Gren, Eds., Sinauer Associates, Sunderland, Mass. (1991)). In certain embodiments, one or more unconventional amino acids may be incorporated into a polypeptide. The term “unconventional amino acid” refers to any amino acid that is not one of the twenty conventional amino acids. The term “non-naturally occurring amino acids” refers to amino acids that are not found in nature. Non-naturally occurring amino acids are a subset of unconventional amino acids. Unconventional amino acids include, but are not limited to, stereoisomers (e.g., D-amino acids) of the twenty conventional amino acids, unnatural amino acids such as α-,α-disubstituted amino acids, N-alkyl amino acids, lactic acid, homoserine, homocysteine, 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and imino acids (e.g., 4-hydroxyproline) known in the art. In the polypeptide notation used herein, the left-hand direction is the amino terminal direction and the right-hand direction is the carboxy-terminal direction, in accordance with standard usage and convention.

A “fragment” of a reference polypeptide refers to a contiguous stretch of amino acids from any portion of the reference polypeptide. A fragment may be of any length that is less than the length of the reference polypeptide.

A “variant” of a reference polypeptide refers to a polypeptide having one or more amino acid substitutions, deletions, or insertions relative to the reference polypeptide. In certain embodiments, a variant of a reference polypeptide has an altered post-translational modification site (i.e., a glycosylation site). In certain embodiments, a variant of a reference polypeptide has altered disulfide connectivity. In certain embodiments, both a reference polypeptide and a variant of a reference polypeptide are specific binding agents. In certain embodiments, both a reference polypeptide and a variant of a reference polypeptide are antibodies.

Variants of a reference polypeptide include, but are not limited to, glycosylation variants. Glycosylation variants include variants in which the number and/or type of glycosylation sites have been altered as compared to the reference polypeptide. In certain embodiments, glycosylation variants of a reference polypeptide comprise a greater or a lesser number of N-linked glycosylation sites than the reference polypeptide. In certain embodiments, an N-linked glycosylation site is characterized by the sequence Asn-X-Ser or Asn-X-Thr, wherein the amino acid residue designated as X may be any amino acid residue except proline. In certain embodiments, glycosylation variants of a reference polypeptide comprise a rearrangement of N-linked carbohydrate chains wherein one or more N-linked glycosylation sites (typically those that are naturally occurring) are eliminated and one or more new N-linked sites are created.

Variants of a reference polypeptide include, but are not limited to, cysteine variants. In certain embodiments, cysteine variants include variants in which one or more cysteine residues of the reference polypeptide are replaced by one or more non-cysteine residues; and/or one or more non-cysteine residues of the reference polypeptide are replaced by one or more cysteine residues. Cysteine variants may be useful, in certain embodiments, when a particular polypeptide must be refolded into a biologically active conformation, e.g., after the isolation of insoluble inclusion bodies. In certain embodiments, cysteine variants of a reference polypeptide have fewer cysteine residues than the reference polypeptide. In certain embodiments, cysteine variants of a reference polypeptide have an even number of cysteines to minimize interactions resulting from unpaired cysteines. In certain embodiments, cysteine variants have more cysteine residues than the native protein.

In certain embodiments, conservative modifications to the heavy and light chains of a particular antibody (and corresponding modifications to the encoding nucleotides) will produce antibodies having functional and chemical characteristics similar to those of the original antibody. In contrast, in certain embodiments, substantial modifications in the functional and/or chemical characteristics of a particular antibody may be accomplished by selecting substitutions in the amino acid sequence of the heavy and light chains that differ significantly in their effect on maintaining (a) the structure of the molecular backbone in the area of the substitution, for example, as a sheet or helical conformation, (b) the charge or hydrophobicity of the molecule at the target site, or (c) the bulk of the side chain.

Certain desired amino acid substitutions (whether conservative or non-conservative) can be determined by those skilled in the art at the time such substitutions are desired. In certain embodiments, amino acid substitutions can be used to identify important residues of particular antibodies, such as those which may increase or decrease the affinity of the antibodies or the effector function of the antibodies.

In certain embodiments, the effects of an antibody may be evaluated by measuring a reduction in the amount of symptoms of the disease. In certain embodiments, the disease of interest may be caused by a pathogen. In certain embodiments, a disease may be established in an animal host by other methods including introduction of a substance (such as a carcinogen) and genetic manipulation. In certain embodiments, effects may be evaluated by detecting one or more adverse events in the animal host. The term “adverse event” includes, but is not limited to, an adverse reaction in an animal host that receives an antibody that is not present in an animal host that does not receive the antibody. In certain embodiments, adverse events include, but are not limited to, a fever, an immune response to an antibody, inflammation, and/or death of the animal host.

Various antibodies specific to an antigen may be produced in a number of ways. In certain embodiments, an antigen containing an epitope of interest may be introduced into an animal host (e.g., a mouse), thus producing antibodies specific to that epitope. In certain instances, antibodies specific to an epitope of interest may be obtained from biological samples taken from hosts that were naturally exposed to the epitope. In certain instances, introduction of human immunoglobulin (Ig) loci into mice in which the endogenous Ig genes have been inactivated offers the opportunity to obtain human monoclonal antibodies (MAbs).

The term “agent” is used herein to denote a chemical compound, a mixture of chemical compounds, a biological molecule, a biological macromolecule, or an extract made from biological materials.

The term “stabilizing agent” refers to an agent that stabilizes a specific binding agent in a composition. A specific binding agent is “stabilized” in a composition if the specific binding agent retains more of its physical stability and/or chemical stability and/or biological activity in a composition comprising a stabilizing agent compared with the composition not comprising the stabilizing agent.

A specific binding agent in a composition contained in a container, e.g., a syringe, is “stabilized” if the specific binding agent retains at least the same or similar physical stability and/or chemical stability and/or biological activity after being subjected to one or more of the laboratory tests which simulate shipping conditions that are discussed in the following documents: Singh, J., S. P. Singh and G. Burgess, Measurement and Analysis of US Truck Vibration for Leaf Spring and Air Ride Suspensions and Development of Test Tests, Packaging Technology and Science, Vol 19, 2006; International Safe Transit Association (ISTA) Resource Book 2006, Test Procedure 3A; Singh, S. P. and J. Marcondes, “Vibration Levels in Commercial Truck Shipments as a Function of Suspension and Payload”, Journal of Testing and Evaluation, ASTM, Vol 20, No. 6, 466-469, 1992; Kipp, William I., Vibration Testing Equivalence, How Many Hours Of Testing Equals How Many Miles Of Transport?, Proceedings of ISTA Con 2000; Rouillard, V., A New Approach to Analyzing and Simulating Shock and Vibration, Proceedings of Dimensions 06, International Safe Transit Association, East Lansing, Mich. 48823, 2006; Singh, S. P., G. Burgess and P. Rojuckarin, “Test Protocol for Simulating Truck and Rail Vibration and Rail Impacts in Shipments of Automotive Engine Racks”, Packaging Technology and Science, Vol. 8, 33-41, 1995; Brandenburg and Lee. (2001). Fundamentals of Packaging Dynamics. L.A.B. Equipment Inc.: Skaneateles, N.Y.; and Singh, S. P., G. Burgess, Marcondes, Jorge A., and Antle, John R., “Measuring the Package Shipping Environment in Refrigerated Ocean Vessels”, Packaging Technology and Science, Vol. 6, 175-181, 1993. A specific binding agent in a composition contained in a container is considered “stabilized” if the specific binding agent retains at least the same or similar physical stability and/or chemical stability and/or biological stability after being subjected to at least one test, even if one or more of those properties is not retained after being subjected to one or more tests. In certain embodiments, the laboratory test includes vibration, shock/drop, and/or pressure changes to simulate air and/or vehicular travel. The laboratory test also includes a control, in which the specific binding agent contained in a container is not subjected to vibration, shock/drop, and/or pressure changes. Following vibration, shock/drop, and/or pressure changes to simulate air and/or vehicular travel, the physical stability and/or chemical stability and/or biological activity of the specific binding agent is determined and compared to the physical stability and/or chemical stability and/or biological activity of the control specific binding agent. In certain embodiments, a specific binding agent is considered to retain at least the same or similar physical stability and/or chemical stability and/or biological activity if the specific binding agent is suitable for use as a pharmaceutical in a human.

When a stabilizing agent is used, the phrase “retains its physical stability” means that a specific binding agent in a composition shows less aggregation and/or precipitation and/or denaturation in a composition comprising a stabilizing agent compared with the composition not comprising the stabilizing agent. The phrase “retains its physical stability” also means a specific binding agent in a composition contained in a container of a certain type, e.g., syringe, shows the same or similar or less aggregation and/or precipitation and/or denaturation after being subjected to one or more of the laboratory tests discussed in paragraph 84, which simulate shipping conditions. A specific binding agent in a composition contained in a container is considered to retain its physical stability if the specific binding agent shows the same or similar or less aggregation and/or precipitation and/or denaturation after being subjected to at least one test, even if one or more of those properties is not the same or similar or less after being subjected to one or more tests. In certain embodiments, a composition contained in a container of a certain type, e.g., syringe, shows the same or similar or less aggregation and/or precipitation and/or denaturation after being subjected to a laboratory test which simulates shipping conditions, followed by subsequent storage under static conditions as the specific binding agent in the composition contained in a container of the same type stored under static conditions and not subjected to a laboratory test which simulates shipping conditions. In certain embodiments, a composition contained in a container subjected to a laboratory test which simulates shipping conditions, and followed by subsequent storage under static conditions is stored at a temperature between 2° C. and 8° C. In certain embodiments, a composition contained in a container subjected to a laboratory test which simulates shipping conditions, and followed by subsequent storage under static conditions is stored at a temperature between 15° C. and 45° C. In certain embodiments, a composition contained in a container stored under static conditions and not subjected to a laboratory test which simulates shipping conditions, is stored in a freezer at a temperature between −20° C. and −80° C. In certain embodiments, a composition contained in a container subjected to a laboratory test which simulates shipping conditions, and followed by subsequent storage under static conditions is stored for at least 1 month to at least 24 months. Exemplary storage periods include, but are not limited to, at least 1 month, at least 3 months, at least 6 months, at least 9 months, at least 12 months, at least 18 months, and at least 24 months. Exemplary methods of determining the amount of aggregation and/or precipitation and/or denaturation of a specific binding agent include, but are not limited to, visual inspection; subvisible particulate counting by light obscuration, for example, by using a HIAC (Royco) instrument; microscopic particle counting; size-exclusion high-performance liquid chromatography (SEC-HPLC), and SDS-PAGE. A specific binding agent in a composition contained in a container is considered to retain its physical stability if the specific binding agent shows the same or similar or less aggregation and/or precipitation and/or denaturation as determined in at least one of those determining methods, even if one or more of those properties is not the same or similar or less as determined in one or more of those determining methods. In certain embodiments, a specific binding agent is considered to show the same or similar or less aggregation and/or precipitation and/or denaturation if the specific binding agent is suitable for use as a pharmaceutical in a human.

When a stabilizing agent is used, the phrase “retains its chemical stability” means that a specific binding agent in a composition shows less chemical alteration in a composition comprising a stabilizing agent compared with the composition not comprising the stabilizing agent. The phrase “retains its chemical stability” also means a specific binding agent in a composition contained in a container of a certain type, e.g., syringe, shows the same or similar or less chemical alteration after being subjected to one or more of the laboratory tests discussed in paragraph 84, which simulate shipping conditions. Examples of chemical alteration include, but are not limited to, size modification, for example, including, but not limited to, clipping. Clipping refers to cleavage of a specific binding agent that results in smaller fragments. In certain embodiments, clipping is a result of proteolysis. Examples of chemical alteration include, but are not limited to, charge alteration, for example, including, but not limited to, deamidation. Examples of chemical alteration include, but are not limited to, hydrophilic/hydrophobic alteration, for example, including, but not limited to, oxidation. Examples of chemical alteration include, but are not limited to, isomerization. A specific binding agent in a composition contained in a container is considered to retain its chemical stability if the specific binding agent shows the same or similar or less of any type of chemical alteration after being subjected to at least one test, even if one or more types of chemical alteration is not the same or similar or less after being subjected to one or more tests. In certain embodiments, a composition contained in a container of a certain type, e.g., syringe, shows the same or similar or less chemical alteration after being subjected to a laboratory test which simulates shipping conditions, followed by subsequent storage under static conditions as the specific binding agent in the composition contained in a container of the same type stored under static conditions and not subjected to a laboratory test which simulates shipping conditions. In certain embodiments, a composition contained in a container subjected to a laboratory test which simulates shipping conditions, and followed by subsequent storage under static conditions is stored at a temperature between 2° C. and 8° C. In certain embodiments, a composition contained in a container subjected to a laboratory test which simulates shipping conditions, and followed by subsequent storage under static conditions is stored at a temperature between 15° C. and 45° C. In certain embodiments, a composition contained in a container stored under static conditions and not subjected to a laboratory test which simulates shipping conditions, is stored in a freezer at a temperature between −20° C. and −80° C. In certain embodiments, a composition contained in a container subjected to a laboratory test which simulates shipping conditions, and followed by subsequent storage under static conditions is stored for at least 1 month to at least 24 months. Exemplary storage periods include, but are not limited to, at least 1 month, at least 3 months, at least 6 months, at least 9 months, at least 12 months, at least 18 months, and at least 24 months. Exemplary methods of determining the amount of chemical alteration of a specific binding agent include, but are not limited to, cation-exchange HPLC, reversed-phase HPLC, SDS-PAGE, and peptide mapping. A specific binding agent in a composition contained in a container is considered to retain its chemical stability if the specific binding agent shows the same or similar or less of any type of chemical alteration as determined in at least one of those determining methods, even if one or more types of chemical alteration is not the same or similar or less as determined in one or more of those determining methods. In certain embodiments, a specific binding agent is considered to show the same or similar or less chemical alteration if the specific binding agent is suitable for use as a pharmaceutical in a human.

When a stabilizing agent is used, the phrase “retains its biological activity” means that a specific binding agent in a composition demonstrates more biological activity at a given time after the composition was prepared in a composition comprising a stabilizing agent compared with the composition not comprising the stabilizing agent. The phrase “retains its biological activity” also means a specific binding agent in a composition contained in a container of a certain type, e.g., syringe, demonstrates at least the same or similar biological activity at a given time after the composition was prepared and after being subjected to one or more of the laboratory tests discussed in paragraph 84, which simulate shipping conditions. A specific binding agent in a composition contained in a container is considered to retain its biological activity if the specific binding agent demonstrates at least the same or similar of any type of biological activity at a given time after the composition was prepared and after being subjected to at least one test, even if one or more types of biological activity is not at least the same or similar at a given time after the composition was prepared and after being subjected to one or more tests. In certain embodiments, a composition contained in a container of a certain type, e.g., syringe, demonstrates at least the same or similar biological activity at a given time after the composition was prepared and after being subjected to a laboratory test which simulates shipping conditions, followed by subsequent storage under static conditions as the specific binding agent in the composition contained in a container of the same type stored under static conditions and not subjected to a laboratory test which simulates shipping conditions. In certain embodiments, a composition contained in a container subjected to a laboratory test which simulates shipping conditions, and followed by subsequent storage under static conditions is stored at a temperature between 2° C. and 8° C. In certain embodiments, a composition contained in a container subjected to a laboratory test which simulates shipping conditions, and followed by subsequent storage under static conditions is stored at a temperature between 15° C. and 45° C. In certain embodiments, a composition contained in a container stored under static conditions and not subjected to a laboratory test which simulates shipping conditions, is stored in a freezer at a temperature between −20° C. and −80° C. In certain embodiments, a composition contained in a container subjected to a laboratory test which simulates shipping conditions, and followed by subsequent storage under static conditions is stored for at least 1 month to at least 24 months. Exemplary storage periods include, but are not limited to, at least 1 month, at least 3 months, at least 6 months, at least 9 months, at least 12 months, at least 18 months, and at least 24 months. In certain embodiments, biological activity is determined by an assay appropriate for determining biological activity. Exemplary assays to determine biological activity of a specific binding agent include, but are not limited to, antigen binding assays and receptor phosphorylation assays. Exemplary antigen binding assays include, but are not limited to, ELISA assays, immunoprecipitation assays, and affinity assays, for example, including, but not limited to, BIAcore® assays. Certain exemplary methods and assays to determine biological activity of specific binding agents to RANKL have been described, e.g., in U.S. Publication No. 2004/0033535, published Feb. 19, 2004. Certain exemplary methods and assays to determine biological activity of specific binding agents to IL-1R1 have been described, e.g., in U.S. Publication No. 2004/0097712, published May 20, 2004. Certain exemplary methods and assays to determine biological activity of specific binding agents to TNF and of specific binding agents to TNF-R have been described, e.g., in U.S. Pat. No. 5,945,397. A specific binding agent in a composition contained in a container is considered to retain its biological activity if the specific binding agent demonstrates at least the same or similar of any type of biological activity at a given time after the composition was prepared as determined in at least one assay, even if one or more types of biological activity at a given time after the composition was prepared is not the same or similar as determined in one or more assays. In certain embodiments, a specific binding agent is considered to demonstrate at least the same or similar biological activity at a given time after the composition was prepared if the specific binding agent is suitable for use as a pharmaceutical in a human.

In certain embodiments, biologically active TNF receptors are capable of binding greater than 0.1 nmoles TNF per nmole receptor. In certain embodiments, biologically active TNF receptors are capable of binding greater than 0.5 nmole TNF per nmole receptor in standard binding assays. Certain exemplary binding assays and methods to determine binding of TNF and TNF-R have been described, e.g., in U.S. Pat. No. 5,945,397.

“Aggregation” refers to the formation of multimers of individual protein molecules through non-covalent or covalent interactions. Aggregation also refers to the formation of particles. Particles may be either subvisible or visible. Subvisible particles are of a size between 2 μM and 100 μM. Visible particles are of a size greater than 100 μM. Aggregation can be reversible or irreversible. In certain instances, when the loss of tertiary structure or partial unfolding occurs, hydrophobic amino acid residues which are typically hidden within the folded protein structure are exposed to the solution. In certain instances, this promotes hydrophobic-hydrophobic interactions between individual protein molecules, resulting in aggregation. Srisailam et al., J Am Chem Soc 124 (9):1884-8 (2002), for example, has determined that certain conformational changes of a protein accompany aggregation, and that certain regions of specific proteins can be identified as particularly responsible for the formation of aggregates. In certain instances, protein aggregation can be induced by heat (Sun et al., J Agric Food Chem 50(6): 1636-42 (2002)), organic solvents (Srisailam et al., supra), and reagents such as SDS and lysophospholipids (Hagihara et al., Biochem 41(3): 1020-6 (2002)). Aggregation can be a significant problem in in vitro protein purification and formulation. In certain instances, after formation of aggregates, solubilization with strong denaturating solutions followed by renaturation and proper refolding may be needed before biological activity is restored.

“Denaturation” refers to an alteration of the three-dimensional structure of a polypeptide. Three-dimensional structure of a polypeptide includes, but is not limited to, secondary structure and tertiary structure. Secondary structure refers to the local conformation of a portion of a polypeptide. Certain exemplary secondary structures include, but are not limited to, α helix; β conformation, β sheet, and β turn. Tertiary structure refers to the overall three-dimensional arrangement of atoms in a polypeptide. In certain instances, tertiary structure includes interactions between amino acids that are located far apart in the linear sequence. In certain instances, the alteration of three-dimensional structure is such that a polypeptide is partially or completely unfolded. In certain instances, the alteration of three-dimensional structure is sufficient to cause a partial or complete loss of function. In certain instances, denaturation can be induced by exposure of a polypeptide to any one or more of the following: heat; pH extremes; organic solvents, including, but not limited to, alcohol and acetone; detergents, including, but not limited to, SDS; and chaotropic reagents, including, but not limited to, urea and guanidine hydrochloride. In certain instances, denaturation of a polypeptide can be induced by exposure of the polypeptide to a surface of a container, for example, including, but not limited to, containers comprising glass, stainless steel, polycarbonate, polytetrafluoroethylene (Teflon®), polyurethane, silicone, polyvinyl chloride, ethylene-vinyl acetate, polyester, and polyolefin. In certain instances, denaturation of a polypeptide can be induced by exposure of the polypeptide to a surface of a container closure, for example, including, but not limited to, container closures comprising silicone oil, butyl rubber, fluorocarbon and tungsten. In certain instances, denaturation of a polypeptide can be induced by exposure of the polypeptide to a phase interface, for example, including, but not limited to an air/liquid interface, an ice/liquid interface, and an aqueous/oil interface. In certain instances, denaturation of certain polypeptides, for example, globular proteins, by exposure to organic solvents, urea, and detergents results in disruption of hydrophobic interactions within the polypeptide. In certain instances, denaturation of a polypeptide by exposure to, for example, pH extremes results in alteration of the net charge of the polypeptide, which causes electrostatic repulsion and disruption of certain hydrogen bonding within the polypeptide.

The term “shipping,” “ships,” or “shipped” refers to transporting a composition in a container in a vehicle, airplane, and/or ship, by any route, for any distance, and at any temperature.

The phrase “stored under static conditions,” or “static storage conditions” refers to keeping a composition in a container in a location without shipping.

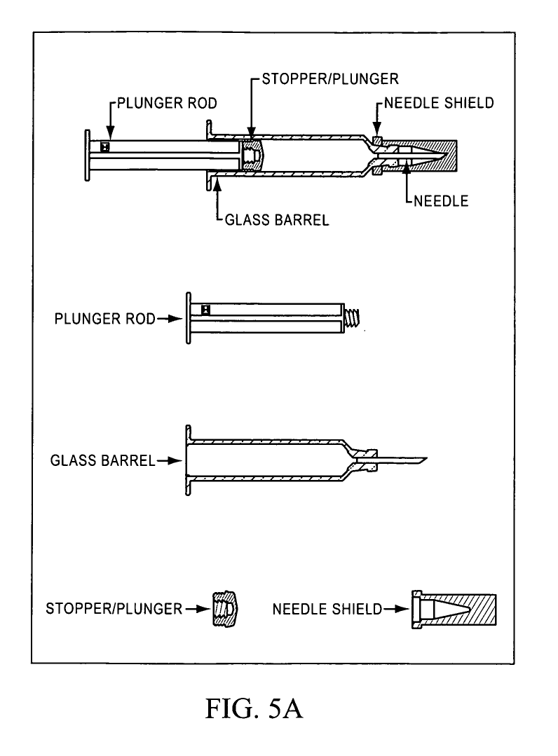

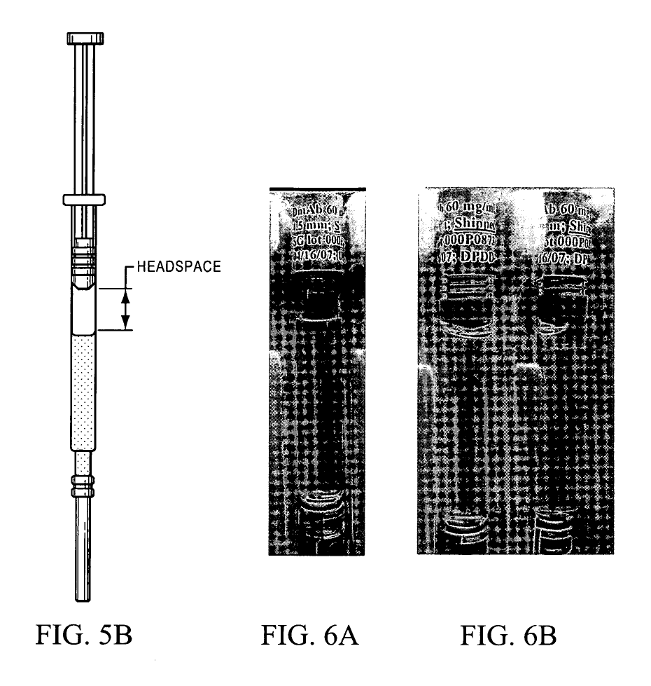

The term “headspace” refers to the space between a liquid in a container and the container closure. See, e.g., FIG. 5 (B) and FIGS. 6 (A) and (B). In certain embodiments, the headspace is of a size such that a meniscus is formed by the liquid in the container, which is visible by eye or by light microscopy. As used herein, a “meniscus” refers to a concave upper surface of a liquid in a container. When the container is in a vertical (upright) position, the meniscus extends across the diameter of a container and no liquid touches the bottom surface of the container closure. In certain embodiments, the headspace is the distance between the top of the meniscus and the bottom surface of the container closure, e.g., the flat body portion in the center of a plunger. In certain embodiments, the headspace is of a size such that a meniscus is not formed, but is of a size such that an air bubble is formed by the liquid in the container, which is visible by eye or by light microscopy. As used herein, an “air bubble” does not extend across the diameter of a container when the container is in a vertical position, and some, but not all, of the liquid touches the bottom surface of the container closure. In certain embodiments, the air bubble is spherical in shape. In certain of those embodiments, the headspace is the diameter of the air bubble. In certain embodiments, the air bubble is not spherical in shape. In certain such embodiments, the air bubble is elliptical in shape. In certain of those embodiments, the headspace is the largest dimension of the air bubble. In certain embodiments, headspace is measured using calipers. In certain such embodiments, a 10× magnifying lens is used with a certified and calibrated caliper. An exemplary caliper is Mitutoyo Series 500, MCN number 900-G1-222. In certain embodiments, headspace is measured using a microscope and microscope ruler. In certain such embodiments, calipers are used to record the distance between the top of the meniscus to the bottom of the flat body of the plunger using calipers. In certain embodiments, the headspace of a prefilled and stoppered syringe is measured with an optical comparator. An exemplary optical comparator is Deltronic DH 216, Horizontal Optical Comparator. In certain such embodiments, measurements are made by placing the syringe in a vertical position and parallel to the optical lens. A magnified image is projected onto a screen for inspection. Calipers on the optical comparator are used to record the distance between the top of the meniscus to the bottom of the flat body of the plunger. In certain embodiments, the headspace is the distance in millimeters from the top of the meniscus to the bottom of the flat body of the plunger.

As used herein, headspace is “minimized” when the headspace measurement is 3.0 mm or less using the caliper and/or microscope measurement methods described above in paragraph 90 and in Example 2 below. A headspace is considered “minimized” if the headspace measurement is 3.0 mm or less using at least one measurement method, even if the measurement is greater than 3.0 mm using one or more other tests. Certain exemplary minimized headspace measurements include, but are not limited to, 2.7 mm or less, or 2.5 mm or less, or 2 mm or less, or 1.5 mm or less, or 1 mm or less, or 0.5 mm or less, or 0.2 mm or less, or 0.1 mm or less, or no detectable headspace. In certain embodiments, the minimized headspace measurement is between 2.5 mm and 3.0 mm, or 2.0 mm and 2.5 mm, or 1.5 mm and 2.0 mm, or 1.0 and 1.5 mm. See, e.g., FIG. 6 (B). In certain embodiments, headspace is minimized when the headspace cannot be measured using the caliper and/or microscope measurement methods described herein. In certain such embodiments, there is no meniscus visible by eye or by light microscopy. In certain such embodiments, there is no air bubble visible by eye or by light microscopy.

A “container closure” refers to a part of a container or container assembly that covers or seals the container. In certain embodiments, the container closure holds a composition inside a container. In certain embodiments, the container closure is impermeable to microbial ingress. Exemplary container closures include, but are not limited to, caps, lids, plungers, and stoppers.

A “prefilled syringe” refers to a container for a composition, for example, a therapeutic composition, in which the container is a syringe, the composition is placed in the syringe prior to administration of the composition to a patient, and the syringe is covered with a syringe closure, for example, but not limited to, a plunger. In certain embodiments, the composition is placed in the syringe in a manufacturing fill facility. In certain embodiments, the syringe is washed and sterilized prior to placing the composition in the syringe. In certain embodiments, the prefilled syringe includes the composition for at least 1 day, or at least 7 days, or at least 14 days, or at least 1 month, or at least 6 months, or at least 1 year, or at least 2 years prior to administration of the composition to a patient. In certain embodiments, the prefilled syringe is subject to storage and/or shipping conditions.

The term “silicone” refers to a lubricant comprising a semi-inorganic polymer based on the structural unit R₂SiO, where R is an organic group. In certain embodiments, the silicone is polydimethylsiloxane, also referred to as silicone oil. In certain embodiments, the internal surface of a syringe barrel, the surface of a syringe plunger, and/or the surface of a syringe needle is coated with silicone. In certain embodiments, other types of containers and/or container closures, including, but not limited to, stopcocks, are coated with silicone. Certain exemplary polydimethylsiloxanes include, but are not limited to, Dow Corning® 360 Medical Fluid, including for example, but not limited to, Dow Corning® 360 Medical Fluid having a viscosity of 350 centistokes, Dow Corning® 360 Medical Fluid having a viscosity of 1000 centistokes, Dow Corning® 360 Medical Fluid having a viscosity of 12,500 centistokes, and Dow Corning® MDX4-4159 fluid. In certain embodiments, silicone oil is sprayed on the surface. In certain embodiments, silicone oil is wiped on the surface. In certain embodiments, silicone oil is baked. In certain embodiments, silicone oil is cross-linked.

A “lubricant” refers to a material that, when applied as a surface coating, reduces friction between moving parts. Certain exemplary lubricants include, but are not limited to, silicone, polytetrafluoroethylene (Teflon®), and TriboGlide® (TriboFilm Research, Inc., Raleigh, N.C.). Certain exemplary coatings of stoppers include, but are not limited to, Omniflex (Helvoet Pharma, Inc., Pennsauken, N.J.), Nanoskin (plasma-coated perfluoropolyether [PFPE] on Helvovet stoppers [formulation FM457] from TriboFilm, Raleigh, N.C.), and Fluorotec® (Daikyo Seiko, Ltd., Sumida-Ku, Tokyo).

The term “baked silicone” refers to silicone, which, after being applied to a container, for example, including, but not limited to, a syringe, is treated with heat thereby promoting binding of silicone to the surface of the container.

The term “cross-linked silicone” refers to a cross-linkable silicone oil which has been subjected to a cross-linking treatment. Cross-linkable silicone oils include, but are not limited to, silicone oils having reactive and/or functional chemical groups enabling cross-linking of the oil. An exemplary commercially available cross-linkable silicone oil includes, but is not limited to, Dow Corning® MDX4-4159. Exemplary cross-linking treatments include, but are not limited to, treatment by irradiation, including for example, but not limited to, exposure to an electron, x-ray, or γ-ray source; and treatment in an ionizing plasma, including for example, but not limited to, oxygen plasma.

The terms “silicone-free material” and “material lacks silicone” refers to material used in the manufacture of a container or a container closure in which no silicone has been added to coat a surface. In certain embodiments, silicone is not detectable as determined in one or more of the following tests: exposing the material to solvent that will extract silicone, and detecting silicone by either (1) an Inductively Coupled Plasma (ICP) assay coupled with Mass Spectrometry (ICP-MS), atomic emission spectroscopy (ICP-AES), or atomic absorption (ICP-AA), as described in Kennan J J, Breen L L, Lane T H, Taylor R B., Methods for detecting silicones in biological matrixes, Analytical Chemistry, 71(15):3054-60, 1999; Mundry T, Surmann P, Schurreit T., Trace analysis of silicone oil in aqueous parenteral formulation and glass containers by graphite furnace atomic absorption spectrometry, Drugs made in Germany, Vol 44, No 2, 47-56, 2001; Carter, J., L. Ebdon, and E. H. Evans, Speciation of silicon and phosphorous using liquid chromatography coupled with sector field high resolution ICP-MS, Microchemical Journal, 2004, 76(1-2): p. 35-41; or Klemens P, Heumann K G., Development of an ICP-HRIDMS method for accurate determination of traces of silicon in biological and clinical samples, Fresenius J Anal Chem, 371:758-763, 2001; or (2) a Fourier Transform Infrared (FTIR) spectroscopic assay, as described in Silverstein, R. M., Bassler, G. C., Morrill, T. C. Spectrometric Identification of Organic Compounds, 5th Ed., 1991; or Güngel, H., Menceoglu, Yildiz, B., Akbulut, O., Fourier Transform Infrared And 1H Nuclear Magnetic Resonance Spectroscopic Findings Of Silicone Oil Removed From Eyes And The Relationship Of Emulsification With Retinotomy And Glaucoma, The Journal Of Retinal And Vitreous Diseases, Vol 25, No 3, 332-338, 2005. Silicon is considered not to be detectable if it is not detectable in at least one of these tests, even if it is detectable in one or more other tests.

The terms “lubricant-free material” and “material lacks lubricant” refers to material used in the manufacture of a container or a container closure in which no lubricant has been added to coat a surface. In certain embodiments, lubricant is not detectable as determined in one or more of the following tests: exposing the material to solvent that will extract lubricant, and detecting lubricant by either (1) an Inductively Coupled Plasma (ICP) assay coupled with Mass Spectrometry (ICP-MS), atomic emission spectroscopy (ICP-AES), or atomic absorption (ICP-AA), as described in Kennan J J, Breen L L, Lane T H, Taylor R B., Methods for detecting silicones in biological matrixes, Analytical Chemistry, 71(15):3054-60, 1999; Mundry T, Surmann P, Schurreit T., Trace analysis of silicone oil in aqueous parenteral formulation and glass containers by graphite furnace atomic absorption spectrometry, Drugs made in Germany, Vol 44, No 2, 47-56, 2001; Carter, J., L. Ebdon, and E. H. Evans, Speciation of silicon and phosphorous using liquid chromatography coupled with sector field high resolution ICP-MS, Microchemical Journal, 2004, 76(1-2): p. 35-41; or Klemens P, Heumann K G., Development of an ICP-HRIDMS method for accurate determination of traces of silicon in biological and clinical samples, Fresenius J Anal Chem, 371:758-763, 2001; or (2) a Fourier Transform Infrared (FTIR) spectroscopic assay, as described in Silverstein, R. M., Bassler, G. C., Morrill, T. C. Spectrometric Identification of Organic Compounds, 5th Ed., 1991; or Güngel, H., Menceoglu, Yildiz, B., Akbulut, O., Fourier Transform Infrared And 1H Nuclear Magnetic Resonance Spectroscopic Findings Of Silicone Oil Removed From Eyes And The Relationship Of Emulsification With Retinotomy And Glaucoma, The Journal Of Retinal And Vitreous Diseases, Vol 25, No 3, 332-338, 2005. Lubricant is considered not to be detectable if it is not detectable in at least one of these tests, even if it is detectable in one or more other tests.

The term “high molecular weight plastic material” refers to a plastic material having a molecular weight of at least 40,000. In certain embodiments, high molecular weight plastic material comprises polymerized cyclic monomers. Certain exemplary high molecular weight plastic materials include, but are not limited to, cyclic olefin copolymer and cyclic olefin polymer.

A “buffering agent” or “buffer” refers to an agent that maintains the pH of a composition within a desired range.

The terms “osteopenic disorder,” “bone loss,” or “bone loss condition” includes, but is not limited to, osteoporosis; including, but not limited to, postmenopausal osteoporosis, endocrine osteoporosis (including, but not limited to, hyperthyroidism, hyperparathyroidism, Cushing's syndrome, and acromegaly), hereditary and congenital forms of osteoporosis (including, but not limited to, osteogenesis imperfecta, homocystinuria, Menkes' syndrome, and Riley-Day syndrome); and osteoporosis due to immobilization of extremities; Paget's disease of bone (osteitis deformans) in adults and juveniles; osteomyelitis, or an infectious lesion in bone, leading to bone loss; hypercalcemia resulting from solid tumors (including, but not limited to, breast, lung and kidney) and hematologic malignancies (including, but not limited to, multiple myeloma, lymphoma and leukemia), idiopathic hypercalcemia, and hypercalcemia associated with hyperthyroidism and renal function disorders; osteopenia following surgery, associated with use of steroids, such as glucocorticoids, and associated with disorders of the small and large intestine and with chronic hepatic and renal diseases; osteonecrosis, or bone cell death, associated with traumatic injury or nontraumatic necrosis; bone loss associated with anemia or inflammatory or autoimmune conditions such as systemic lupus erythematosus and rheumatoid arthritis, and periodontal disease.

In addition to those bone loss conditions, certain cancers, including those which metastasize to bone or are resident in bone are known to increase osteoclast activity and induce bone resorption. Such cancers include, but are not limited to, breast cancer, prostate cancer, and multiple myeloma. In certain instances, these cancers are known to produce factors that result in the over-expression of RANKL in the bone, and lead to increased osteoclast numbers and activity. Accordingly, bone loss disorders include, but are not limited to, breast cancer, prostate cancer, and solid tumors that have metastasized to bone or are capable of metastasizing to bone; multiple myeloma; and giant cell tumor of bone. Other bone loss conditions include, but are not limited to, chemotherapy-induced bone loss in patients with metastatic and non-metastatic cancer, including, but not limited to, breast cancer and prostate cancer. In certain instances, bone loss occurs during hormone ablative therapy, such as, for example, but not limited, with adjuvant aromatase inhibitors.

A disease or medical condition is considered to be an “interleukin-1 (IL-1) mediated disease” if the naturally-occurring or experimentally-induced disease or medical condition is associated with elevated levels of IL-1 in bodily fluids or tissue or if cells or tissues taken from the body produce elevated levels of IL-1 in culture. Elevated levels of IL-1 include, for example, but are not limited to, levels that exceed those normally found in a particular cell or tissue; and any detectable level of IL-1 in a cell or tissue that normally does not express a detectable level of IL-1. In certain instances, IL-1 mediated diseases are also recognized by either one or both of the following additional two conditions: (1) pathological findings associated with the disease or medical condition mimicked experimentally in animals by administration of IL-1 or by experimental conditions resulting in up-regulation of expression of IL-1; and (2) a pathology induced in experimental animal models of the disease or medical condition inhibited or abolished by treatment with agents that inhibit the action of IL-1. In certain IL-1 mediated diseases, at least two of the three conditions are met. In certain IL-1 mediated diseases, all three conditions are met.

Exemplary acute and chronic IL-1-mediated diseases include, but are not limited to, the following: acute pancreatitis; amyotrophic lateral sclerosis (ALS); Alzheimer's disease; cachexia/anorexia, including, but not limited to, AIDS-induced cachexia; asthma and other pulmonary diseases; atherosclerosis; autoimmune vasculitis; chronic fatigue syndrome; Clostridium associated illnesses, including, but not limited to, Clostridium-associated diarrhea; coronary conditions and indications, including, but not limited to, congestive heart failure, coronary restenosis, myocardial infarction, myocardial dysfunction (e.g., related to sepsis), and coronary artery bypass graft; cancer, including, but not limited to, multiple myeloma and myelogenous (e.g., AML or CML) and other leukemias, as well as tumor metastasis; diabetes (e.g., insulin-dependent diabetes); endometriosis; fever; fibromyalgia; glomerulonephritis; graft versus host disease/transplant rejection; hemorrhagic shock; hyperalgesia; inflammatory bowel disease; inflammatory conditions of a joint, including, but not limited to, osteoarthritis, psoriatic arthritis and rheumatoid arthritis; inflammatory eye disease, as may be associated with, e.g., corneal transplant; ischemia, including cerebral ischemia (e.g., brain injury as a result of trauma, epilepsy, hemorrhage or stroke, each of which may lead to neurodegeneration); Kawasaki's disease; learning impairment; lung diseases (e.g., ARDS); multiple sclerosis; myopathies (e.g., muscle protein metabolism, especially in sepsis); neurotoxicity (e.g., as induced by HIV); osteoporosis; pain, including, but not limited to, cancer-related pain; Parkinson's disease; periodontal disease; pre-term labor; psoriasis; reperfusion injury; septic shock; side effects from radiation therapy; temporal mandibular joint disease; sleep disturbance; uveitis and inflammatory conditions resulting from strain, sprain, cartilage damage, trauma, orthopedic surgery, infection or other disease processes.

A disease or medical condition is considered to be an “TNF-mediated disease” if the naturally-occurring or experimentally-induced disease or medical condition is associated with elevated levels of TNF in bodily fluids or tissue or if cells or tissues taken from the body produce elevated levels of TNF in culture. Elevated levels of TNF include, for example, but are not limited to, levels that exceed those normally found in a particular cell or tissue; and any detectable level of TNF in a cell or tissue that normally does not express a detectable level of TNF. In certain instances, TNF-mediated diseases are also recognized by either one or both of the following additional two conditions: (1) pathological findings associated with the disease or medical condition mimicked experimentally in animals by administration of TNF or by experimental conditions resulting in up-regulation of expression of TNF; and (2) a pathology induced in experimental animal models of the disease or medical condition inhibited or abolished by treatment with agents that inhibit the action of TNF. In certain TNF-mediated diseases, at least two of the three conditions are met. In certain TNF-mediated diseases, all three conditions are met.

Exemplary acute and chronic TNF-mediated diseases include, but are not limited to, cachexia, septic shock, AIDS, cardiomyopathy, autoimmune diseases, and inflammatory diseases, including, but not limited to, rheumatoid arthritis, psoriatic arthritis, juvenile rheumatoid arthritis, ankylosing spondylitis, and plaque psoriasis.

A specific binding agent “substantially inhibits binding” of a ligand to a receptor when an excess of specific binding agent reduces the quantity of receptor bound to the ligand by at least about 20%, 40%, 60%, 80%, 85%, or more (as measured in an in vitro competitive binding assay). In certain embodiments, a specific binding agent is an antibody. In certain such embodiments, an antibody substantially inhibits binding of RANKL to RANK, or substantially inhibits binding of IL-1 to IL-1R1. In certain embodiments, a specific binding agent is a soluble fusion polypeptide. In certain such embodiments, a soluble fusion polypeptide substantially inhibits binding of TNF to TNF-R.

The term “cancer” includes, but is not limited to solid tumors and hematologic malignancies. Exemplary cancers include, but are not limited to, breast cancer, colorectal cancer, gastric carcinoma, glioma, head and neck squamous cell carcinoma, hereditary and sporadic papillary renal carcinoma, leukemia, lymphoma, Li-Fraumeni syndrome, malignant pleural mesothelioma, melanoma, multiple myeloma, non-small cell lung carcinoma, osteosarcoma, ovarian cancer, pancreatic cancer, prostate cancer, small cell lung cancer, synovial sarcoma, thyroid carcinoma, Giant Cell Tumor, and transitional cell carcinoma of urinary bladder.

The term “pharmaceutical agent or drug” as used herein refers to a chemical compound or composition capable of inducing a desired therapeutic effect when properly administered to a patient. As used herein, a therapeutic effect may or may not include a prophylactic effect.

The term “modulator,” as used herein, is a compound that changes or alters the activity or function of a molecule. For example, a modulator may cause an increase or decrease in the magnitude of a certain activity or function of a molecule compared to the magnitude of the activity or function observed in the absence of the modulator. In certain embodiments, a modulator is an inhibitor, which decreases the magnitude of at least one activity or function of a molecule. Certain exemplary activities and functions of a molecule include, but are not limited to, binding affinity, enzymatic activity, and signal transduction. Certain exemplary inhibitors include, but are not limited to, proteins, peptides, antibodies, peptibodies, carbohydrates or small organic molecules. Peptibodies are described in, e.g., U.S. Pat. No. 6,660,843 and PCT Publication No. WO 01/83525.

As used herein, “substantially pure” means an object species is the predominant species present (i.e., on a molar basis it is more abundant than any other individual species in the composition). In certain embodiments, a substantially purified fraction is a composition wherein the object species comprises at least about 50 percent (on a molar basis) of all macromolecular species present. In certain embodiments, a substantially pure composition will comprise more than about 80%, 85%, 90%, 95%, or 99% of all macromolecular species present in the composition. In certain embodiments, the object species is purified to essential homogeneity (contaminant species cannot be detected in the composition by conventional detection methods) wherein the composition consists essentially of a single macromolecular species.

The term “patient” includes human and animal subjects.

### Certain Exemplary Specific Binding Agents

In certain instances, TNF is released by activated macrophages and T cells, inducing a wide variety of effects on a large number of cell types. In certain instances, TNF plays a role in regulating the normal immune response, as well as in various pathological and disease states. Certain such pathological and disease states include, but are not limited to, systemic toxicity associated with sepsis, pathogenesis of AIDS, and various autoimmune and inflammatory diseases, including, but not limited to, rheumatoid arthritis, juvenile rheumatoid arthritis, ankylosing spondylitis, and plaque psoriasis.

TNF proteins initiate their biological effect on cells, in certain instances, by binding to specific TNF receptor (TNF-R) proteins expressed on the plasma membrane of a TNF-responsive cell. Therefore, in certain instances, a reduction in TNF-mediated cellular responses may reduce the severity of arthritic, immune, autoimmune, and/or inflammatory disorders. According to certain embodiments, specific binding agents to TNF are used to treat immune, autoimmune, and/or inflammatory disorders, including, but not limited to, those mentioned above.

In certain embodiments, specific binding agents to TNF are soluble TNF-R. In certain embodiments, nucleotide sequences encoding soluble TNF-R, and corresponding amino acid sequences, are provided. In certain embodiments, soluble TNF-R is selected from huTNF-RΔ235, huTNF-RΔ185 and huTNF-RΔ163. See U.S. Pat. No. 5,945,397. In certain embodiments, soluble TNF-R is monovalent. Monovalent soluble TNF-R possesses single TNF-R binding sites for TNF ligand. In certain embodiments, soluble TNF-R is polyvalent. Polyvalent soluble TNF-R possesses multiple TNF-R binding sites for TNF ligand. In certain such embodiments, soluble TNF-R is bivalent. In certain such embodiments, bivalent soluble TNF-R comprises two tandem repeats of huTNF-RΔ235 separated by a linker region. In certain embodiments, a purified human soluble TNF-R capable of binding TNF is provided.

In certain embodiments, specific binding agents to TNF are soluble TNF-R fusion polypeptides. In certain embodiments, soluble TNF-R fusion polypeptides are polyvalent. In certain such embodiments, soluble TNF-R fusion polypeptides are bivalent (also referred to as dimeric). In certain embodiments, soluble TNF-R fusion polypeptides comprise soluble TNF-R fused to Fc.

Certain exemplary soluble TNF-R and soluble TNF-R fusion polypeptides and methods of making and using such polypeptides are described in U.S. Pat. No. 5,945,397 and Mohler et al., J. Immunol. 151:1548-1561 (1993). In certain such embodiments, a purified soluble human TNF-R fusion polypeptide is provided. In certain such embodiments, a purified soluble human TNF-R fusion polypeptide is sTNFR:Fc as described in Mohler et al., J. Immunol. 151:1548-1561 (1993), or etanercept, which is sold under the tradename Enbrel®, discussed in the Examples below.

In certain instances, RANKL is involved in the formation of osteoclasts. In certain instances, RANKL binds to a receptor, RANK, which increases osteoclast activity. In certain instances, increased osteoclast activity correlates with certain osteopenic disorders, including post-menopausal osteoporosis, Paget's disease, lytic bone metastases, and rheumatoid arthritis. Therefore, in certain instances, a reduction in RANKL activity may result in a decrease in osteoclast activity and may reduce the severity of osteopenic disorders. According to certain embodiments, specific binding agents to RANKL are used treat osteopenic disorders, including by not limited to, those mentioned above.

In certain embodiments, specific binding agents to RANKL are fully human monoclonal antibodies. In certain embodiments, nucleotide sequences encoding heavy and light chain immunoglobulin molecules, and corresponding amino acid sequences, particularly sequences corresponding to the variable regions are provided. In certain embodiments, sequences corresponding to complementarity determining regions (CDR's), specifically from CDR1 through CDR3, are provided. According to certain embodiments, a hybridoma cell line expressing such an immunoglobulin molecule is provided. According to certain embodiments, a hybridoma cell line expressing such a monoclonal antibody is provided. According to certain embodiments, a Chinese Hamster Ovary (CHO) cell line expressing such a monoclonal antibody is provided. In certain embodiments, a purified human monoclonal antibody to human RANKL is provided.

Certain exemplary antibodies to RANKL (also referred to as OPGL) and methods of making and using such antibodies are described in U.S. Publication No. 2004/0033535, published Feb. 19, 2004. In certain such embodiments, a purified human monoclonal antibody to human RANKL is provided. See, e.g., U.S. Publication No. 2004/0033535. In certain such embodiments, a purified human monoclonal antibody to human RANKL is αRANKL-1, discussed in the Examples below.

In certain instances, IL-1, a cytokine, is involved in the inflammatory response. In certain instances, IL-1 binds to a receptor, IL-1R1, followed by binding to IL-1RAcP. Those events are followed by signal transduction resulting in the induction of a cellular response, which, in certain instances, leads to inflammation. In certain instances, inflammation is associated with injuries resulting from mechanical damage, infection, or antigenic stimulation. In certain instances, inflammatory reactions are expressed pathologically. Such conditions arise, in certain instances, when the inflammation is expressed in an exaggerated manner, is inappropriately stimulated, or persists after the injurious agent is removed. Exemplary pathological conditions mediated by IL-1 include, but are not limited to, rheumatoid arthritis and osteoarthritis. Therefore, in certain instances, a reduction in IL-1 mediated signal transduction activity may result in a decrease in cellular responses leading to inflammation and may reduce the severity of arthritic and other inflammatory disorders. According to certain embodiments, specific binding agents to IL-1R1 are used treat inflammatory disorders, including by not limited to, those mentioned above.

In certain embodiments, specific binding agents to IL-1R1 are fully human monoclonal antibodies. In certain embodiments, nucleotide sequences encoding heavy and light chain immunoglobulin molecules, and corresponding amino acid sequences, particularly sequences corresponding to the variable regions are provided. In certain embodiments, sequences corresponding to complementarity determining regions (CDR's), specifically from CDR1 through CDR3, are provided. According to certain embodiments, a hybridoma cell line expressing such an immunoglobulin molecule is provided. According to certain embodiments, a hybridoma cell line expressing such a monoclonal antibody is provided. According to certain embodiments, a Chinese Hamster Ovary (CHO) cell line expressing such a monoclonal antibody is provided. In certain embodiments, a monoclonal antibody is selected from at least one of 15C4, 26F5, and 27F2. In certain embodiments, a purified human monoclonal antibody to human IL-1R1 is provided.

Certain exemplary antibodies to IL-1R1 and methods of making and using such antibodies are described in U.S. Publication No. 2004/0097712, published May 20, 2004. In certain such embodiments, a purified human monoclonal antibody to human IL-1R1 is provided. In certain such embodiments, a purified human monoclonal antibody to human IL-1R1 has a light chain variable region of SEQ ID NO:12 and a heavy chain variable region of SEQ ID NO:10 as set forth in U.S. Publication No. 2004/0097712; or alternatively a light chain variable region of SEQ ID NO:12 and a heavy chain variable region of SEQ ID NO:14 as set forth in U.S. Publication No. 2004/0097712; or alternatively a light chain variable region of SEQ ID NO:18 and a heavy chain variable region of SEQ ID NO:16 as set forth in U.S. Publication No. 2004/0097712.

In certain embodiments, a human monoclonal antibody to human RANKL and/or a human monoclonal antibody to IL-1R1 is a fully human monoclonal antibody. Certain fully human monoclonal antibodies can be obtained from engineered mouse strains as follows. One can engineer mouse strains deficient in mouse antibody production with large fragments of the human Ig loci in anticipation that such mice would produce human antibodies in the absence of mouse antibodies. Large human Ig fragments may preserve the large variable gene diversity as well as the proper regulation of antibody production and expression. By exploiting the mouse machinery for antibody diversification and selection and the lack of immunological tolerance to human proteins, the reproduced human antibody repertoire in these mouse strains may yield high affinity fully human antibodies against any antigen of interest, including human antigens. Using the hybridoma technology, antigen-specific human MAbs with the desired specificity may be produced and selected. Certain exemplary methods are described in WO 98/24893, U.S. Pat. No. 5,545,807, EP 546073B1, and EP 546073A1.

In certain embodiments, one may use constant regions from species other than human along with the human variable region(s). In certain embodiments, one may use constant regions from human along with variable region(s) from species other than human.

### Certain Exemplary Antibody Structure

Naturally occurring antibody structural units typically comprise a tetramer. Each such tetramer typically is composed of two identical pairs of polypeptide chains, each pair having one full-length light chain (in certain embodiments, about 25 kDa) and one full-length heavy chain (in certain embodiments, about 50-70 kDa).

The amino-terminal portion of each chain typically includes a variable region (V_{H} in the heavy chain and V_{L} in the light chain) of about 100 to 110 or more amino acids that typically is responsible for antigen recognition. The carboxy-terminal portion of each chain typically defines a constant region (C_{H} domains in the heavy chain and C_{L} in the light chain) that may be responsible for effector function. Antibody effector functions include activation of complement and stimulation of opsonophagocytosis. Human light chains are typically classified as kappa and lambda light chains. Heavy chains are typically classified as mu, delta, gamma, alpha, or epsilon, and define the antibody's isotype as IgM, IgD, IgG, IgA, and IgE, respectively. IgG has several subclasses, including, but not limited to, IgG1, IgG2, IgG3, and IgG4. IgM has subclasses including, but not limited to, IgM1 and IgM2. IgA is similarly subdivided into subclasses including, but not limited to, IgA1 and IgA2. Within full-length light and heavy chains, typically, the variable and constant regions are joined by a “J” region of about 12 or more amino acids, with the heavy chain also including a “D” region of about 10 more amino acids. See, e.g., Fundamental Immunology Ch. 7 (Paul, W., ed., 2nd ed. Raven Press, N.Y. (1989)). The variable regions of each light/heavy chain pair typically form the antigen binding site.

The variable regions typically exhibit the same general structure of relatively conserved framework regions (FR) joined by three hypervariable regions, also called complementarity determining regions or CDRs. The CDRs from the heavy and light chains of each pair typically are aligned by the framework regions, which may enable binding to a specific epitope. From N-terminal to C-terminal, both light and heavy chain variable regions typically comprise the domains FR1, CDR1, FR2, CDR2, FR3, CDR3, and FR4. The assignment of amino acids to each domain is typically in accordance with the definitions of Kabat Sequences of Proteins of Immunological Interest (National Institutes of Health, Bethesda, Md. (1987 and 1991)), or Chothia & Lesk J. Mol. Biol. 196:901-917 (1987); Chothia et al. Nature 342:878-883 (1989).

As discussed in the “Certain Definitions” section above, there are several types of antibody fragments. Exemplary antibody fragments include, but are not limited to, Fab fragment, Fab′ fragment, F(ab)₂ molecule, Fv molecule, scFv, maxibody, and Fc fragment.

In certain embodiments, functional domains, C_{H}1, C_{H}2, C_{H}3, and intervening sequences can be shuffled to create a different antibody constant region. For example, in certain embodiments, such hybrid constant regions can be optimized for half-life in serum, for assembly and folding of the antibody tetramer, and/or for improved effector function. In certain embodiments, modified antibody constant regions may be produced by introducing single point mutations into the amino acid sequence of the constant region and testing the resulting antibody for improved qualities, e.g., one or more of those listed above.

In certain embodiments, an antibody of one isotype is converted to a different isotype by isotype switching without losing its specificity for a particular target molecule. Methods of isotype switching include, but are not limited to, direct recombinant techniques (see e.g., U.S. Pat. No. 4,816,397) and cell-cell fusion techniques (see e.g., U.S. Pat. No. 5,916,771), among others. In certain embodiments, an antibody can be converted from one subclass to another subclass using techniques described above or otherwise known in the art without losing its specificity for a particular target molecule, including, but not limited to, conversion from an IgG2 subclass to an IgG1, IgG3, or IgG4 subclass.

### Certain Bispecific or Bifunctional Antibodies

A bispecific or bifunctional antibody typically is an artificial hybrid antibody having two different heavy/light chain pairs and two different binding sites. Bispecific antibodies may be produced by a variety of methods including, but not limited to, fusion of hybridomas or linking of Fab′ fragments. See, e.g., Songsivilai & Lachmann Clin. Exp. Immunol. 79: 315-321 (1990), Kostelny et al. J. Immunol. 148:1547-1553 (1992).

### Certain Preparation of Antibodies

In certain embodiments, antibodies can be expressed in cell lines other than hybridoma cell lines. In certain embodiments, sequences encoding particular antibodies, including chimeric antibodies, can be used for transformation of a suitable mammalian host cell. According to certain embodiments, transformation can be by any known method for introducing polynucleotides into a host cell, including, for example packaging the polynucleotide in a virus (or into a viral vector) and transducing a host cell with the virus or by transfecting a vector using procedures known in the art, as exemplified by U.S. Pat. Nos. 4,399,216; 4,912,040; 4,740,461; and 4,959,455.

In certain embodiments, an expression vector comprises one or more polynucleotide sequences discussed herein, including, but not limited to, polynucleotide sequences encoding one or more antibodies. In certain embodiments, a method of making a polypeptide comprising producing the polypeptide in a cell comprising any of the above expression vectors in conditions suitable to express the polynucleotide contained therein to produce the polypeptide is provided.

In certain embodiments, an expression vector expresses an antibody heavy chain. In certain embodiments, an expression vector expresses an antibody light chain. In certain embodiments, an expression vector expresses both an antibody heavy chain and an antibody light chain. In certain embodiments, a method of making an antibody comprising producing the antibody in a cell comprising at least one of expression vectors in conditions suitable to express the polynucleotides contained therein to produce the antibody is provided.

In certain embodiments, the transfection procedure used may depend upon the host to be transformed. Certain methods for introduction of heterologous polynucleotides into mammalian cells are known in the art and include, but are not limited to, dextran-mediated transfection, calcium phosphate precipitation, polybrene mediated transfection, protoplast fusion, electroporation, encapsulation of the polynucleotide(s) in liposomes, and direct microinjection of the DNA into nuclei.

Certain mammalian cell lines available as hosts for expression are known in the art and include, but are not limited to, many immortalized cell lines available from the American Type Culture Collection (ATCC), including but not limited to Chinese hamster ovary (CHO) cells, E5 cells, HeLa cells, baby hamster kidney (BHK) cells, monkey kidney cells (COS), human hepatocellular carcinoma cells (e.g., Hep G2), NS0 cells, SP20 cells, Per C6 cells, 293 cells, and a number of other cell lines. In certain embodiments, cell lines may be selected through determining which cell lines have high expression levels and produce antibodies with constitutive antigen binding properties.

In certain embodiments, the vectors that may be transfected into a host cell comprise control sequences that are operably linked to a polynucleotide encoding an antibody. In certain embodiments, control sequences facilitate expression of the linked polynucleotide, thus resulting in the production of the polypeptide encoded by the linked polynucleotide. In certain embodiments, the vector also comprises polynucleotide sequences that allow chromosome-independent replication in the host cell. Exemplary vectors include, but are not limited to, plasmids (e.g., BlueScript, puc, etc.), cosmids, and YACS.

### Certain Expression of Recombinant Polypeptides

In certain embodiments, recombinant expression vectors are used to amplify or express DNA encoding polypeptides, for example, including, but not limited to TNF-R. In certain embodiments, recombinant expression vectors are replicable DNA constructs which have synthetic or cDNA-derived DNA fragments encoding mammalian TNF-R or bioequivalent analogs operably linked to suitable transcriptional or translational regulatory elements derived from mammalian, microbial, viral or insect genes. Various recombinant expression vectors suitable for expression of synthetic or cDNA-derived DNA fragments encoding polypeptides are well known to one skilled in the art. Certain exemplary recombinant expression vectors are described in U.S. Pat. No. 5,945,397.

In certain embodiments, transformed host cells are cells which have been transformed or transfected with TNF-R vectors constructed using recombinant DNA techniques. Transformed host cells ordinarily express TNF-R, but host cells transformed for purposes of cloning or amplifying TNF-R DNA do not need to express TNF-R. In certain embodiments, expressed TNF-R will be deposited in the cell membrane or secreted into the culture supernatant, depending on the TNF-R DNA selected. Exemplary host cells for expression of mammalian TNF-R include, but are note limited to, prokaryotes, yeast or higher eukaryotic cells, wherein the expression of TNF-R is under the control of appropriate promoters. Prokaryotes include gram negative or gram positive organisms, for example E. coli or bacilli. Higher eukaryotic cells include, but are not limited to, established cell lines of mammalian origin. In certain embodiments, cell-free translation systems could also be employed to produce mammalian TNF-R using RNAs derived from the DNA constructs containing TNF-R. Certain exemplary cloning and expression vectors for use with bacterial, fungal, yeast, and mammalian cellular hosts are described by Pouwels et al. (Cloning Vectors: A Laboratory Manual, Elsevier, N.Y., 1985).

In certain embodiments, prokaryotic expression hosts are used for expression of TNF-R. In certain embodiments, prokaryotic expression vectors generally comprise one or more phenotypic selectable markers, for example a gene encoding proteins conferring antibiotic resistance or supplying an auxotrophic requirement, and an origin of replication recognized by the host to ensure amplification within the host. Exemplary prokaryotic hosts for transformation include E. coli, Bacillus subtilis, Salmonella typhimurium, and various species within the genera Pseudomonas, Streptomyces, and Staphyolococcus. Various prokaryotic expression vectors and methods of use are well known to one skilled in the art. Certain prokaryotic expression vectors are described in U.S. Pat. No. 5,945,397.

In certain embodiments, recombinant TNF-R proteins are expressed in yeast hosts, for example, Saccharomyces cerevisiae, and yeast of other genera, such as Pichia or Kluyveromyces. Various yeast expression vectors and methods of use are well known to one skilled in the art. Certain exemplary yeast expression vectors and methods of use are described in R. Hitzeman et al., European Patent Application Publication No. 0073657, and in Sherman et al., Laboratory Course Manual for Methods in Yeast Genetics, Cold Spring Harbor Laboratory, Cold Spring Harbor, N.Y., 1986.

In certain embodiments, mammalian or insect cell culture systems are employed to express recombinant protein. Examples of suitable mammalian host cell lines include, but are not limited to, the COS-7 lines of monkey kidney cells, described by Gluzman (Cell 23:175, 1981), and other cell lines capable of expressing an appropriate vector including, for example, L cells, C127, 3T3, Chinese hamster ovary (CHO), HeLa and BHK cell lines. Various mammalian and insect cell culture systems and methods of use are well known to one skilled in the art. Certain such exemplary systems are described in U.S. Pat. No. 5,945,397.

In certain embodiments, recombinant expression vectors comprising TNF-R cDNAs are stably integrated into a host cell's DNA. In certain embodiments, elevated levels of expression product is achieved by selecting for cell lines having amplified numbers of vector DNA. In certain embodiments, cell lines having amplified numbers of vector DNA are selected, for example, by transforming a host cell with a vector comprising a DNA sequence which encodes an enzyme which is inhibited by a known drug. In certain embodiments, the vector also comprises a DNA sequence which encodes the desired protein, e.g., TNF-R. In certain embodiments, the host cell is co-transformed with a second vector which comprises the DNA sequence encoding the desired protein, e.g., TNF-R. In certain embodiments, the transformed or co-transformed host cells are then cultured in increasing concentrations of the known drug, thereby selecting for drug-resistant cells which may contain amplified copies of the vector encoding the enzyme as well as the vector DNA encoding the desired protein (TNF-R) in the host cell's DNA.

Certain exemplary systems for such co-amplification include, include but are not limited to, use the gene for dihydrofolate reductase (DHFR), which can be inhibited by the drug methotrexate (MTX); and use of the gene for glutamine synthetase (GS), which is responsible for the synthesis of glutamate and ammonia using the hydrolysis of ATP to ADP and phosphate to drive the reaction. Those systems are well known to those skilled in the art. In addition, the GS co-amplification system, appropriate recombinant expression vectors and cells lines, are described in the following PCT applications: WO 87/04462, WO 89101036, WO 89/10404 and WO 86/05807.

In certain embodiments, recombinant proteins are expressed by co-amplification of DHFR or GS in a mammalian host cell, such as Chinese Hamster Ovary (CHO) cells, or alternatively in a murine myeloma cell line, such as SP2/0-Ag14 or NS0 or a rat myeloma cell line, such as YB2/3.0-Ag20, disclosed in PCT applications WO/89/10404 and WO 86/05807.

Certain additional eukaryotic vectors for expression of TNF-R DNA, including the vector, pCAV/NOT, are described in U.S. Pat. No. 5,945,397.

### Purification of Recombinant TNF-R

In certain embodiments, purified mammalian TNF receptors or analogs are prepared by culturing suitable host/vector systems to express the recombinant translation products of the TNF-R DNAs, which are then purified from culture media or cell extracts.

In certain embodiments, supernatants from systems which secrete recombinant protein into culture media are first concentrated using a commercially available protein concentration filter, for example, an Amicon or Millipore Pellicon ultrafiltration unit. In certain embodiments, following the concentration step, the concentrate is applied to a suitable purification matrix. Exemplary purification matrices include, but are not limited to, a TNF, lectin or antibody polypeptide bound to a suitable support; an anion exchange resin comprising, for example, pendant diethylaminoethyl (DEAE) groups, wherein the matrix is acrylamide, agarose, dextran, cellulose or other types commonly employed in protein purification; a cation exchange resin, comprising various insoluble matrices comprising sulfopropyl or carboxymethyl groups.

In certain embodiments, one or more reversed-phase high performance liquid chromatography (RP-HPLC) steps employing hydrophobic RP-HPLC media, e.g., silica gel having pendant methyl or other aliphatic groups, are employed to further purify a TNF-R composition. In certain embodiments, some or all of the foregoing purification steps, in various combinations, are employed to provide a homogeneous recombinant protein.

In certain embodiments, recombinant protein produced in bacterial culture is typically isolated by initial extraction from cell pellets, followed by one or more concentration, salting-out, aqueous ion exchange or size exclusion chromatography steps. In certain embodiments, high performance liquid chromatography (HPLC) is employed for final purification steps. In certain embodiments, microbial cells employed in expression of recombinant mammalian TNF-R are disrupted by any convenient method, for example, freeze-thaw cycling, sonication, mechanical disruption, or use of cell lysing agents.

In certain embodiments, yeast cells, which express mammalian TNF-R as a secreted protein, are fermented. An exemplary method to purify secreted recombinant protein resulting from a large-scale fermentation is discussed in Urdal et al. (J. Chromatog. 296:171, 1984).

### Certain Specific Binding Agent Compositions

In certain embodiments, a composition comprising at least one specific binding agent, at least one stabilizing agent, and a buffering agent is provided. In certain such embodiments, the composition further comprises at least one additional pharmaceutical agent. In certain embodiments, the specific binding agent is a specific binding agent to RANKL, a specific binding agent to TNF, and/or a specific binding agent to IL-1R1. In certain embodiments, the specific binding agent is a specific binding agent to RANKL, wherein the specific binding agent to RANKL is an antibody which specifically binds RANKL. In certain embodiments, the antibody is αRANKL-1. In certain embodiments, the specific binding agent is a specific binding agent to TNF, wherein the specific binding agent to TNF is a soluble TNF receptor. In certain embodiments, the soluble TNF receptor is sTNFR:Fc. In certain embodiments, the specific binding agent is a specific binding agent to IL-1R1, wherein the specific binding agent is an antibody which specifically binds IL-1R1. In certain embodiments, the antibody is selected from 15C4, 26F5 and 27F2 as described in U.S. Publication No. 2004/0097712.

In certain embodiments, the at least one specific binding agent to RANKL is at a concentration of 1 mg/ml to 150 mg/ml. In certain such embodiments, the at least one specific binding agent to RANKL is an antibody which specifically binds RANKL. In certain embodiments, the antibody is αRANKL-1. Certain exemplary concentrations of the at least one specific binding agent to RANKL include, but are not limited to, 30 mg/ml, 60 mg/ml, 70 mg/ml, 105 mg/ml, and 120 mg/ml. In certain embodiments, compositions will include more than one different specific binding agent to RANKL. In certain such embodiments, the more than one specific binding agents to RANKL bind more than one epitope.

In certain embodiments, the at least one specific binding agent to TNF is at a concentration of 1 mg/ml to 150 mg/ml. In certain such embodiments, the at least one specific binding agent to TNF is present at a concentration of 50 mg/ml. In certain embodiments, compositions will include more than one different specific binding agent to TNF. In certain such embodiments, the more than one specific binding agents to TNF bind more than one epitope. Exemplary formulations for a specific binding agent to TNF, including soluble TNFR:Fc, can be found in U.S. Patent Publication No. 2007-0243185, incorporated herein by reference in its entirety.

In certain embodiments, the at least one specific binding agent to IL-1R1 is at a concentration of 1 mg/ml to 200 mg/ml. In certain such embodiments, the at least one specific binding agent to IL-1R1 is an antibody which specifically binds IL-1R1. In certain embodiments, the antibody is selected from 15C4, 26F5 or 27F2 as described in U.S. Publication No. 2004/0097712. Certain exemplary concentrations of the at least one specific binding agent to IL-1R1 include, but are not limited to, 30 mg/ml, 70 mg/ml, 100 mg/ml, and 150 mg/ml. In certain embodiments, compositions will include more than one different specific binding agent to IL-1R1. In certain such embodiments, the more than one specific binding agents to IL-1R1 bind more than one epitope.

In certain embodiments, the pH of a composition comprising a buffering agent is below 6.6. In certain embodiments, the pH of a composition comprising a buffering agent is between 5.5 and 6.5. In certain such embodiments, the pH is 6.3. In certain embodiments, the pH of a composition comprising a buffering agent is between 4.5 and 5.5. In certain such embodiments, the pH is 5.2. Exemplary buffering agents include, but are not limited to, acetate, histidine, phosphate, glutamate, and propionate. In certain embodiments, the concentration of a buffering agent ranges from 1 mM to 50 mM. In certain embodiments, the concentration of the buffering agent is 25 mM. In certain embodiments, the concentration of the buffering agent is 10 mM.

In certain embodiments, the composition further comprises at least one sugar. As used herein, the term “sugar” refers to monosaccharides such as glucose and mannose, or polysaccharides including disaccharides such as sucrose and lactose, as well as sugar derivatives including sugar alcohols and sugar acids. Sugar alcohols include, but are not limited to, mannitol, xylitol, erythritol, threitol, sorbitol and glycerol. A non-limiting example of a sugar acid is L-gluconate. Certain exemplary sugars include, but are not limited to, trehalose and glycine. In certain embodiments, a sugar is provided at a concentration between 0.5% and 9.5%. In certain embodiments, a sugar is 1% sucrose. In certain embodiments, a sugar is 5.0% sorbitol.

In certain embodiments, the composition further comprises at least one surfactant. As used herein, the term “surfactant” refers to a surface-active agent comprising a hydrophobic portion and a hydrophilic portion. Examples of surfactants include, but are not limited to, detergents and bile acid salts. In certain instances, surfactants are categorized as anionic, nonionic, zwitterionic, or cationic, depending on whether they comprise one or more charged group. Nonionic surfactants contain non-charged polar groups and have no charge. Certain exemplary nonionic surfactants include, but are not limited to, polyethylene glycol (PEG), including, but not limited to, PEG 8000, and polysorbate, including but not limited to, polysorbate 80 (Tween® 80) and polysorbate 20 (Tween® 20), Triton X-100, polyoxypropylene-polyoxyethylene esters (Pluronic®), and NP-40. In certain embodiments, the surfactant is provided at a concentration between 0.001% and 1.0%. In certain embodiments, the surfactant is provided at a concentration between 0.003% and 0.3%. In certain embodiments, the surfactant is provided at a concentration of 0.01%. In certain embodiments, the surfactant is provided at a level below the critical micelle concentration (CMC) of the surfactant. In certain such embodiments, the composition comprises a human monoclonal antibody to human IL-1R1 and polysorbate 20, which has a CMC of 0.007%, and the concentration of polysorbate 20 is 0.004%. In certain embodiments, the surfactant is provided at a level above the CMC of the surfactant. In certain such embodiments, the composition comprises αRANKL-1 and polysorbate 20, which has a CMC of 0.007%, and the concentration of polysorbate 20 is 0.01%.

In certain embodiments, a composition comprising at least one specific binding agent, at least one stabilizing agent, and a buffering agent provides stabilization of at least one specific binding agent. In certain embodiments, the specific binding is a specific binding agent to RANKL, a specific binding agent to TNF, and/or a specific binding agent to IL-1R1. In certain embodiments, the specific binding agent is a specific binding agent to RANKL, wherein the specific binding agent to RANKL is an antibody which specifically binds RANKL. In certain embodiments, the antibody is αRANKL-1. In certain embodiments, the specific binding agent is a specific binding agent to TNF, wherein the specific binding agent to TNF is a soluble TNF receptor. In certain embodiments, the soluble TNF receptor is sTNFR:Fc. In certain embodiments, the specific binding agent is a specific binding agent to IL-1R1, wherein the specific binding agent to IL-1R1 is an antibody which specifically binds IL-1R1. In certain embodiments, the antibody is selected from 15C4, 26F5 and 27F2 as described in U.S. Publication No. 2004/0097712. In certain embodiments, the composition provides stabilization with respect to formation of fewer aggregates and/or dimers. In certain embodiments, the composition provides stabilization with respect to formation of fewer chemically altered forms.

In certain embodiments, the presence and degree of aggregation and/or chemically altered forms of a particular protein molecule in a sample can be determined by suitable methods known in the art, such as size exclusion chromatography (SEC), for example, also known as gel filtration chromatography or molecular sieving chromatography. In certain embodiments, a suitable method for determining the presence of aggregates and/or chemically altered forms in a sample is gel electrophoresis under non-denaturing conditions. The “gel” refers to a matrix of water and a polymer such as agarose or polymerized acrylamide. These methods separate molecules on the basis of the size of the molecule compared to the size of the pores of the gel. Certain other methods of measuring aggregation and/or chemically altered forms include, but are not limited to, hydrophobic interaction chromatography (HIC) and high performance liquid chromatography (HPLC). HPLC provides a separation based on any one of adsorption, ion exchange, size exclusion, HIC, or reverse phase chromatography. HIC separates native proteins on the basis of their surface hydrophobicity between the hydrophobic moieties of the protein and insoluble, immobilized hydrophobic groups on the matrix. Generally, the protein preparation in a high salt buffer is loaded on the HIC column. The salt in the buffer interacts with water molecules to reduce the solvation of the proteins in solution, thereby exposing hydrophobic regions in the protein which are then adsorbed by the hydrophobic groups on the matrix. The more hydrophobic the molecule, the less salt is needed to promote binding. Usually, a decreasing salt gradient is used to elute proteins from a column. As the ionic strength decreases, the exposure of the hydrophilic regions of the protein increases and proteins elute from the column in order of increasing hydrophobicity. See, for example, Protein Purification, 2d Ed., Springer-Verlag, New York, 176-179 (1988). In certain embodiments, the separations are improved through the use of high-resolution columns and decreased column retention times. See, for example, Chicz et al., Methods in Enzymology 182, pp. 392-421 (1990). Additional exemplary methods for monitoring protein stability are found in Lee, V., ed. Peptide and Protein Drug Delivery (Marcel Dekker, Inc., New York, N.Y., 1991). In certain embodiments, protein stability is measured at a certain temperature for a certain period of time. In certain embodiments, a specific binding agent to RANKL, a specific binding agent to TNF, and/or a specific binding agent to IL-1R1 is stabilized in a composition stored at room temperature (between 21° C. and 29° C.). Exemplary storage times include, but are not limited to, at least 1 month, at least 3 months, at least 6 months, at least 9 months, at least 12 months, at least 18 months, and at least 24 months. In certain embodiments, a specific binding agent to RANKL, a specific binding agent to TNF, and/or a specific binding agent to IL-1R1 is stabilized in a composition stored between 2° C. and 8° C. Exemplary storage time include, but are not limited to, at least 6 months, at least 9 months, at least 12 months, at least 18 months, and at least 24 months.

In certain embodiments, a specific binding agent to RANKL, a specific binding agent to TNF, and/or a specific binding agent to IL-1R1 is prepared, purified, and formulated as a liquid pharmaceutical composition. In certain embodiments, after preparation and purification, a specific binding agent to RANKL, a specific binding agent to TNF, and/or a specific binding agent to IL-1R1 is stored prior to formulation. In certain such embodiments, the specific binding agent to RANKL, the specific binding agent to TNF, and/or the specific binding agent to IL-1R1 is frozen, for example, at −20° C. or lower. In certain such embodiments, the specific binding agent to RANKL, the specific binding agent to TNF, and/or the specific binding agent to IL-1R1 is thawed at room temperature for further formulation. In certain embodiments, a liquid pharmaceutical formulation comprises a therapeutically effective amount a specific binding agent to RANKL, a specific binding agent to TNF, and/or a specific binding agent to IL-1R1. In certain embodiments, the amount of specific binding agent to RANKL, specific binding agent to TNF, and/or specific binding agent to IL-1R1 to formulate in a formulation will be determined by one skilled in the art, depending upon, for example, the route of administration and desired dose volume. In certain embodiments, the pharmaceutical formulation comprises a specific binding agent to RANKL at a concentration of 1 mg/ml to 150 mg/ml. In certain such embodiments, the specific binding agent to RANKL is an antibody which specifically binds RANKL. In certain embodiments, the antibody is αRANKL-1. In certain embodiments, the pharmaceutical formulation comprises a specific binding agent to TNF at a concentration of 1 mg/ml to 150 mg/ml. In certain such embodiments, the specific binding agent to TNF is a soluble TNF receptor. In certain embodiments, the soluble TNF receptor is sTNFR:Fc. In certain embodiments, the pharmaceutical formulation comprises a specific binding agent to IL-1R1 at a concentration of 1 mg/ml to 200 mg/ml. In certain such embodiments, the specific binding agent to IL-1R1 is an antibody which specifically binds IL-1R1. In certain embodiments, the antibody is selected from 15C4, 26F5, and 27F2 as described in U.S. Publication No. 2004/0097712. In certain embodiments, a pharmaceutical formulation comprises a therapeutically effective amount a specific binding agent to RANKL and a buffer that maintains the pH of the formulation below 6.6. In certain embodiments, a buffer maintains the pH of the formulation between 4.5 and 5.5. In certain such embodiments, a buffer maintains the pH of the formulation at 5.2. In certain embodiments, a pharmaceutical formulation comprises a therapeutically effective amount a specific binding agent to IL-1R1 and a buffer that maintains the pH of the formulation below 6.6. In certain embodiments, a buffer maintains the pH of the formulation between 4.5 and 5.5. In certain such embodiments, a buffer maintains the pH of the formulation at 5.0. In certain embodiments, a pharmaceutical formulation comprises a therapeutically effective amount a specific binding agent to TNF and a buffer that maintains the pH of the formulation between 5.5 and 6.5. In certain embodiments, a buffer maintains the pH of the formulation at 6.3.

In certain embodiments, specific binding agents including, but not limited to, antibodies and soluble polypeptides, which bind to a particular protein and block interaction with other binding compounds may have therapeutic use. In this application, when discussing the use of antibodies and soluble polypeptides to treat diseases or conditions, such use may include use of compositions comprising antibodies or soluble polypeptides; and/or combination therapies comprising antibodies or soluble polypeptides and one or more additional active ingredients. When antibodies or soluble polypeptides are used to “treat” a disease or condition, such treatment may or may not include prevention of the disease or condition.

In certain embodiments, a specific binding agent including, but not limited to, an antibody or a soluble polypeptide, is administered alone. In certain embodiments, an antibody or soluble polypeptide is administered prior to the administration of at least one other therapeutic agent. In certain embodiments, an antibody or soluble polypeptide is administered concurrent with the administration of at least one other therapeutic agent. In certain embodiments, an antibody or soluble polypeptide is administered subsequent to the administration of at least one other therapeutic agent. Exemplary therapeutic agents, include, but are not limited to, at least one cancer therapy agent. Exemplary cancer therapy agents include, but are not limited to, radiation therapy and chemotherapy.

In certain embodiments, pharmaceutical compositions comprising specific binding agents, e.g., antibodies or soluble polypeptides, can be administered in combination therapy, i.e., combined with other agents. Exemplary agents include, but are not limited to, in vitro synthetically prepared chemical compositions, antibodies, antigen binding regions, radionuclides, and combinations and conjugates thereof. In certain embodiments, an agent may act as an agonist, antagonist, allosteric modulator, or toxin. In certain embodiments, an agent may act to inhibit or stimulate its target (e.g., receptor or enzyme activation or inhibition), and thereby promote cell death or arrest cell growth. In certain embodiments, the combination therapy comprises a specific binding agent to RANKL, a specific binding agent to TNF, and/or a specific binding agent to IL-1R1, in combination with at least one anti-angiogenic agent. In certain embodiments, the specific binding agent to RANKL is an antibody which specifically binds RANKL. In certain embodiments, the antibody is αRANKL-1. In certain embodiments, the specific binding agent to TNF is a soluble TNF receptor. In certain embodiments, the soluble TNF receptor is sTNFR:Fc. In certain embodiments, the specific binding agent to IL-1R1 is an antibody which specifically binds IL-1R1. In certain embodiments, the antibody is selected from 15C4, 26F5 and 27F2 as described in U.S. Publication No. 2004/0097712.

Exemplary chemotherapy treatments include, but are not limited to anti-neoplastic agents including, but not limited to, alkylating agents including, but not limited to: nitrogen mustards; nitrosoureas; ethylenimines/methylmelamine; alkyl sulfonates; antimetabolites; pyrimidine analogs; purine analogs; natural products, including, but not limited to, antimitotic drugs, vinca alkaloids, podophyllotoxins; antibiotics; enzymes; biological response modifiers; miscellaneous agents, including, but not limited to, platinum coordination complexes; anthracenediones; substituted urea; methylhydrazine derivatives; adrenocortical suppressants; hormones and antagonists.

Exemplary cancer therapies, which may be administered with a specific binding agent to RANKL, a specific binding agent to TNF, and/or a specific binding agent to IL-1R1, also include, but are not limited to, targeted therapies. Examples of targeted therapies include, but are not limited to, use of therapeutic antibodies. Exemplary therapeutic antibodies, include, but are not limited to, mouse, mouse-human chimeric, CDR-grafted, humanized and fully human antibodies, and synthetic antibodies, including, but not limited to, those selected by screening antibody libraries. Exemplary antibodies include, but are not limited to, those which bind to cell surface proteins Her2, CDC20, CDC33, mucin-like glycoprotein, VEGF, and epidermal growth factor receptor (EGFR) present on tumor cells, and optionally induce a cytostatic and/or cytotoxic effect on tumor cells displaying these proteins.

In certain embodiments, cancer therapy agents are anti-angiogenic agents which decrease angiogenesis. In certain embodiments, cancer therapy agents are angiogenesis inhibitors.

In certain embodiments, a specific binding agent to RANKL, a specific binding agent to TNF, and/or a specific binding agent to IL-1R1 may be administered prior to, concurrent with, and subsequent to treatment with a cancer therapy agent. In certain embodiments, a specific binding agent to RANKL, a specific binding agent to TNF, and/or a specific binding agent to IL-1R1 may be administered prophylactically to prevent or mitigate the onset of bone loss by metastatic cancer. In certain embodiments, a specific binding agent to RANKL, a specific binding agent to TNF, and/or a specific binding agent to IL-1R1 may be administered for the treatment of an existing condition of bone loss due to metastasis.

Exemplary cancers include, but are not limited to, breast cancer, colorectal cancer, gastric carcinoma, glioma, head and neck squamous cell carcinoma, hereditary and sporadic papillary renal carcinoma, leukemia, lymphoma, Li-Fraumeni syndrome, malignant pleural mesothelioma, melanoma, multiple myeloma, non-small cell lung carcinoma, osteosarcoma, ovarian cancer, pancreatic cancer, prostate cancer, small cell lung cancer, synovial sarcoma, thyroid carcinoma, and transitional cell carcinoma of urinary bladder.

In certain embodiments, a specific binding agent to RANKL, a specific binding agent to TNF, and/or a specific binding agent to IL-1R1 may be used alone or with at least one additional therapeutic agent for the treatment of cancer. In certain embodiments, a specific binding agent to RANKL, a specific binding agent to TNF, and/or a specific binding agent to IL-1R1 is used in conjunction with a therapeutically effective amount of an additional therapeutic agent.

In certain embodiments, a specific binding agent to RANKL, a specific binding agent to TNF, and/or a specific binding agent to IL-1R1 is used with one or more particular therapeutic agents to treat various cancers. In certain embodiments, a specific binding agent to RANKL, a specific binding agent to TNF, and/or a specific binding agent to IL-1R1 is used with one or more particular therapeutic agents to treat or prevent malaria. In certain embodiments, a specific binding agent to RANKL, a specific binding agent to TNF, and/or a specific binding agent to IL-1R1 is used with one or more particular therapeutic agents to treat or prevent proliferative diabetic retinopathy. In certain embodiments, in view of the condition and the desired level of treatment, two, three, or more agents may be administered. In certain embodiments, such agents may be provided together by inclusion in the same formulation. In certain embodiments, such agents and a specific binding agent to RANKL, a specific binding agent to TNF, and/or a specific binding agent to IL-1R1 may be provided together by inclusion in the same formulation. In certain embodiments, such agents may be formulated separately and provided together by inclusion in a treatment kit. In certain embodiments, such agents and a specific binding agent to RANKL, a specific binding agent to TNF, and/or a specific binding agent to IL-1R1 may be formulated separately and provided together by inclusion in a treatment kit. In certain embodiments, such agents may be provided separately. In certain embodiments, when administered by gene therapy, the genes encoding protein agents and/or a specific binding agent to RANKL, a specific binding agent to TNF, and/or a specific binding agent to IL-1R1 may be included in the same vector. In certain embodiments, the genes encoding protein agents and/or a specific binding agent to RANKL, a specific binding agent to TNF, and/or a specific binding agent to IL-1R1 may be under the control of the same promoter region. In certain embodiments, the genes encoding protein agents and/or a specific binding agent to RANKL, a specific binding agent to TNF, and/or a specific binding agent to IL-1R1 may be in separate vectors.

It is understood that the response by individual patients to the aforementioned medications or combination therapies may vary, and an appropriate efficacious combination of drugs for each patient may be determined by his or her physician.

In certain embodiments, pharmaceutical compositions comprising a specific binding agent to RANKL, a specific binding agent to TNF, and/or a specific binding agent to IL-1R1 together with a pharmaceutically acceptable diluent, carrier, solubilizer, emulsifier, preservative and/or adjuvant are provided.

In certain embodiments, pharmaceutical compositions comprising a specific binding agent to RANKL, a specific binding agent to TNF, and/or a specific binding agent to IL-1R1 and a therapeutically effective amount of at least one additional therapeutic agent, together with a pharmaceutically acceptable diluent, carrier, solubilizer, emulsifier, preservative and/or adjuvant are provided.

In certain embodiments, therapies comprising a specific binding agent to RANKL, a specific binding agent to TNF, and/or a specific binding agent to IL-1R1 and at least one serine protease inhibitor, and methods of treatment using such therapies are provided. In certain embodiments, a therapy comprises a specific binding agent to RANKL, a specific binding agent to TNF, and/or a specific binding agent to IL-1R1, a serine protease inhibitor, and at least one additional agent described herein.

In certain instances, a disturbance of the protease/protease inhibitor balance can lead to protease-mediated tissue destruction, including, but not limited to, tumor invasion of normal tissue leading to metastasis.

In certain embodiments, a specific binding agent to RANKL, a specific binding agent to TNF, and/or a specific binding agent to IL-1R1 may be used with at least one therapeutic agent for inflammation. In certain embodiments, a specific binding agent to RANKL, a specific binding agent to TNF, and/or a specific binding agent to IL-1R1 may be used with at least one therapeutic agent for an immune disorder. Certain exemplary therapeutic agents for inflammation are described, e.g., in C. A. Dinarello and L. L. Moldawer Proinflammatory and Anti-Inflammatory Cytokines in Rheumatoid Arthritis: A Primer for Clinicians Third Edition (2001) Amgen Inc. Thousand Oaks, Calif.

In certain embodiments, pharmaceutical compositions include more than one different specific binding agent to RANKL, specific binding agent to TNF, and/or specific binding agent to IL-1R1. In certain such embodiments, the more than one specific binding agents to RANKL bind more than one epitope. In certain such embodiments, the more than one specific binding agents to TNF bind more than one epitope. In certain such embodiments, the more than one specific binding agents to IL-1R1 bind more than one epitope.

In certain embodiments, liquid compositions comprising one or more specific binding agent to RANKL, one or more specific binding agent to TNF, and/or one or more specific binding agent to IL-1R1 are prepared as aqueous or nonaqueous solutions or suspensions for subsequent administration to a patient.

In certain embodiments, materials for compositions are nontoxic to recipients at the dosages and concentrations employed.

In certain embodiments, the pharmaceutical composition contains formulation materials for modifying, maintaining or preserving, for example, the pH, osmolarity, viscosity, clarity, color, isotonicity, odor, sterility, stability, rate of dissolution or release, adsorption or penetration of the composition. Exemplary formulation materials include, but are not limited to, oils, vitamins, salts, amino acids (including, but not limited to, nonpolar amino acids (including, but not limited to, alanine, valine, leucine, isoleucine, proline, methionine, phenylalanine, or tryptophan)); antimicrobials; antioxidants (including, but not limited to, ascorbic acid, sodium sulfite or sodium hydrogen-sulfite); buffers (including, but not limited to, acetate, histidine, phosphate, citrate, or propionate); bulking agents (including, but not limited to, mannitol or glycine); chelating agents (including, but not limited to, ethylenediamine tetraacetic acid (EDTA)); complexing agents (including, but not limited to, caffeine, polyvinylpyrrolidone, beta-cyclodextrin or hydroxypropyl-beta-cyclodextrin); fillers; sugar or sugar alcohols (including, but not limited to, monosaccharides, disaccharides, polysaccharides, or water soluble glycans); other carbohydrates, for example, saccharides or glucans (including, but not limited to, fructose, glucose, mannose, sorbose, xylose, maltose, sucrose, lactose, dextran, pullulan, dextrin, α and β cyclodextrin, soluble starch, hydroxyethyl starch, carboxymethylcellulose, or mixtures thereof); sugar alcohols (including, but not limited to, mannitol or sorbitol); proteins (including, but not limited to, serum albumin, gelatin or immunoglobulins); coloring, flavoring and diluting agents; emulsifying agents; hydrophilic polymers (including, but not limited to, polyvinylpyrrolidone, including, but not limited to, polyvinylpyrrolidone with an average molecular weight between 2,000 and 3,000, or polyethylene glycol, including, but not limited to, polyethylene glycol with an average molecular weight between 3,000 and 5,000); low molecular weight polypeptides; salt-forming counterions (including, but not limited to, sodium); preservatives (including, but not limited to, benzalkonium chloride, benzoic acid, salicylic acid, thimerosal, phenethyl alcohol, methylparaben, propylparaben, chlorhexidine, sorbic acid or hydrogen peroxide); solvents (including, but not limited to, glycerin or propylene glycol); suspending agents; surfactants or wetting agents (including, but not limited to, polyoxypropylene-polyoxyethylene esters (Pluronic®), PEG, sorbitan esters, polysorbates including, but not limited to, polysorbate 20, polysorbate 80, triton, tromethamine, lecithin, cholesterol, tyloxapal); stabilizing agents (including, but not limited to, nonpolar amino acids); tonicity enhancing agents (including, but not limited to, alkali metal halides, for example, sodium or potassium chloride, mannitol sorbitol); delivery vehicles; diluents; excipients and/or pharmaceutical adjuvants. (Remington's Pharmaceutical Sciences, 18^{th} Edition, A. R. Gennaro, ed., Mack Publishing Company (1990).

In certain embodiments, a specific binding agent to RANKL, a specific binding agent to TNF, and/or a specific binding agent to IL-1R1 is linked to a half-life extending vehicle known in the art. Such vehicles include, but are not limited to, the Fc domain, polyethylene glycol (PEG), polyoxyethylated polyols, and dextran. Such vehicles and methods are described, e.g., in U.S. Pat. Nos. 4,179,337; 4,495,285; 4,609,546; 4,766,106; 6,660,843; and published PCT Application No. WO 99/25044. In certain instances, PEG is soluble in water at room temperature and has the general formula: R(O—CH₂—CH₂)ₙO—R where R is hydrogen, or a protective group, including, but not limited to, an alkyl or alkanol group, and where “n” is a positive integer. In certain embodiments, the protective group has between 1 and 8 carbons. In certain such embodiments, the protective group is methyl. In certain embodiments, “n” is between 1 and 1,000. In certain embodiments, PEG has an average molecular weight between 1,000 and 40,000. Those ranges and any ranges discussed in this application include the endpoints and all values between the endpoints. In certain embodiments, PEG has at least one hydroxy group. In certain such embodiments, the hydroxy group is a terminal hydroxy group. In certain such embodiments, the terminal hydroxy group is activated by N-hydroxysuccinimide to react with a free amino group on a specific binding agent to RANKL, a specific binding agent to TNF, and/or a specific binding agent to IL-1R1 to form a covalently conjugated molecule. In certain embodiments, the type and amount of the reactive groups may be varied to achieve a covalently conjugated PEG/specific binding agent. Preparation of conjugated PEG molecules is within the skill of the art.

In certain embodiments, a half-life extending vehicle is polyoxyethylated polyol. Exemplary polyoxyethylated polyols include, but are not limited to, polyoxyethylated sorbitol, polyoxyethylated glucose, and polyoxyethylated glycerol (POG). In certain embodiments, POG has an average molecular weight between 1,000 and 40,000. That range and any ranges discussed in this application include the endpoints and all values between the endpoints. Certain exemplary structures of POG are found, for example, in Knauf et al., J. Biol. Chem. 263:15064-15070 (1988). Certain exemplary POG conjugates are found, for example, in U.S. Pat. No. 4,766,106.

In certain embodiments, the optimal pharmaceutical composition will be determined by one skilled in the art depending upon, for example, the intended route of administration, delivery format and desired dosage. See, for example, Remington's Pharmaceutical Sciences, supra. In certain embodiments, such compositions may influence the physical state, stability, rate of in vivo release and rate of in vivo clearance of the antibodies of the invention.

In certain embodiments, the primary vehicle or carrier in a pharmaceutical composition is aqueous in nature. For example, in certain embodiments, a suitable vehicle or carrier may be water for injection, physiological saline solution or artificial cerebrospinal fluid, possibly supplemented with other materials common in compositions for parenteral administration. In certain embodiments, the vehicle or carrier is sterile. In certain embodiments, additional components are included. Exemplary additional components include, but are not limited to, fixed oils; polyethylene glycols; glycerin; propylene glycol and other synthetic solvents; antibacterial agents including, but not limited to, benzyl alcohol and methyl parabens; antioxidants including, but not limited to, ascorbic acid and sodium bisulfite; and chelating agents including, but not limited to ethylenediaminetetraacetic acid. In certain embodiments, neutral buffered saline or saline mixed with serum albumin are further exemplary vehicles. In certain embodiments, pharmaceutical compositions comprise Tris buffer of about pH 7.0-8.5, or acetate buffer of about pH 5.0-5.5, or glutamate buffer of about pH 5.0-5.5, or succinate buffer of about pH 5.0-5.5, or histidine buffer of about pH 5.0-5.5, or aspartate buffer of about pH 5.0-5.5, or phosphate buffer of about pH 6.0-6.5, which may further include sucrose, sorbitol or a suitable substitute therefore. In certain embodiments, pharmaceutical compositions are self-buffering. See, e.g., International Application No.: PCT/US2006/022599, published on Dec. 28, 2006. In certain embodiments, a composition comprising a specific binding agent to RANKL, a specific binding agent to TNF, and/or a specific binding agent to IL-1R1, with or without at least one additional therapeutic agents, may be prepared for storage by mixing the selected composition having the desired degree of purity with optional formulation agents (Remington's Pharmaceutical Sciences, supra) in the form of an aqueous solution. In certain embodiments, a pharmaceutical composition is enclosed in a container. Exemplary containers include, but are not limited to, an ampoule, disposable syringe, including, but not limited to, disposable syringe suitable for prefilling, and multiple dose vial made of glass or plastic. In certain embodiments, a composition comprising a specific binding agent to RANKL, a specific binding agent to TNF, and/or a specific binding agent to IL-1R1 is contained in a prefilled syringe. In certain embodiments, the specific binding agent to RANKL is an antibody which specifically binds RANKL. In certain embodiments, the antibody is αRANKL-1. In certain embodiments, the specific binding agent to TNF is a soluble TNF receptor. In certain embodiments, the soluble TNF receptor is sTNFR:Fc. In certain embodiments, the specific binding agent to IL-1R1 is an antibody which specifically binds IL-1R1. In certain embodiments, the antibody is selected from 15C4, 26F5 and 27F2 as described in U.S. Publication No. 2004/0097712. Exemplary syringes suitable for prefilling are described, for example, in U.S. Pat. No. 5,607,400. Syringes suitable for prefilling are available commercially from various sources, for example, Daikyo Seiko, Ltd (Tokyo, Japan), Becton-Dickinson (Franklin Lakes, N.J.), Bunder Glass (Düsseldorf, Germany), and Schott-Form a Vitrum (Lebanon, Pa.).

In certain embodiments, pharmaceutical compositions can be selected for parenteral delivery. Exemplary parenteral delivery includes, but is not limited to, intravenous, intramuscular, intradermal, or subcutaneous administration. In certain embodiments, the compositions may be selected for delivery through the digestive tract, such as orally. The preparation of such pharmaceutically acceptable compositions is within the skill of the art.

In certain embodiments, the formulation components are present in concentrations that are acceptable to the site of administration. In certain embodiments, a pharmaceutical composition comprises a therapeutically effective amount a specific binding agent to RANKL, a specific binding agent to TNF, and/or a specific binding agent to IL-1R1 and a buffer. In certain embodiments, the specific binding agent to RANKL is an antibody which specifically binds RANKL. In certain embodiments, the antibody is αRANKL-1. In certain embodiments, the specific binding agent to TNF is a soluble TNF receptor. In certain embodiments, the soluble TNF receptor is sTNFR:Fc. In certain embodiments, the specific binding agent to IL-1R1 is an antibody which specifically binds IL-1R1. In certain embodiments, the antibody is selected from 15C4, 26F5 and 27F2 as described in U.S. Publication No. 2004/0097712. In certain embodiments, buffers are used to maintain the composition at physiological pH or at a slightly lower pH. In certain embodiments, buffers are between pH 5.5 and pH 8.0. In certain embodiments, buffers are between pH 5.5 and pH 6.5. In certain embodiments, buffers are between pH 4.5 and pH 5.5. Exemplary buffers include, but are not limited to, acids and/or salts thereof, including, but not limited to, succinic acid or succinate, citric acid or citrate, acetic acid or acetate, tartaric acid or tartarate, phosphoric acid or phosphate, propionic acid or propionate, gluconic acid or gluconate, glutamic acid or glutamate, histidine, glycine, aspartic acid or aspartate, maleic acid or maleate, and malic acid or malate buffers. In certain instances, a “salt” refers to an electrically-neutral substance formed between an anion of an acid and an oppositely charged ion. In certain such instances, the oppositely charged ion is referred to as a “counterion.” Exemplary counterions include, but are not limited to, sodium, potassium, ammonium, calcium, and magnesium. In certain embodiments, the concentration of buffer in a formulation is between 1 mM and 50 mM. In certain embodiments, the concentration of buffer in a formulation is between 5 mM and 30 mM. In certain embodiments, the concentration of buffer in a formulation is between 10 mM and 25 mM. Those ranges and any ranges discussed in this application include the endpoints and all values between the endpoints. In certain embodiments, the concentration of buffer in a formulation is 10 mM. In certain embodiments, the concentration of buffer in a formulation is 25 mM.

In certain embodiments, the pharmaceutical formulation comprises a specific binding agent to RANKL, a specific binding agent to TNF, and/or a specific binding agent to IL-1R1 at a concentration of 1 mg/ml to 200 mg/ml and a buffer. In certain embodiments, the buffer is at a concentration between 1 mM and 50 mM, and the pH of the formulation is below 6.6. In certain such embodiments, the pharmaceutical formulation comprises a specific binding agent to RANKL at a concentration of 60 mg/ml and a buffer at a concentration of 10 mM, and the pH of the formulation is 5.2. In certain embodiments, the specific binding agent to RANKL is an antibody which specifically binds RANKL. In certain embodiments, the antibody is αRANKL-1. In certain such embodiments, the pharmaceutical formulation comprises a specific binding agent to TNF at a concentration of 50 mg/ml and a buffer at a concentration of 25 mM, and the pH of the formulation is 6.3. In certain embodiments, the specific binding agent to TNF is a soluble TNF receptor. In certain embodiments, the soluble TNF receptor is sTNFR:Fc.

In certain embodiments, a pharmaceutical formulation comprises a therapeutically effective amount a specific binding agent to RANKL, a specific binding agent to TNF, and/or a specific binding agent to IL-1R1 and a buffer. In certain embodiments, the buffer is a phosphate buffer or an acetate buffer, at a concentration that maintains the pH of the formulation below 6.6. In certain embodiments, the pH of the formulation is between 4.0 and 6.0. The term “phosphate buffer” refers to a buffer comprising a salt of phosphoric acid. The term “acetate buffer” refers to a buffer comprising a salt of acetic acid. In certain embodiments, the phosphate or acetate counterion is sodium. In certain such embodiments, the buffer is sodium phosphate or sodium acetate. Other exemplary counterions include, but are not limited to, potassium, ammonium, calcium, and magnesium. In certain embodiments, the concentration of the phosphate buffer or acetate buffer in the formulation is between 1 mM and 50 mM. In certain embodiments, the concentration of the phosphate buffer or acetate buffer in the formulation is between 5 mM and 30 mM. In certain embodiments, the concentration of the phosphate buffer or acetate buffer in the formulation is between 10 mM and 25 mM. Those ranges and any ranges discussed in this application include the endpoints and all values between the endpoints. In certain embodiments, the concentration of the phosphate buffer or acetate buffer in the formulation is 10 mM. In certain embodiments, the concentration of the phosphate buffer or acetate buffer in the formulation is 25 mM. In certain embodiments, the pharmaceutical formulation comprises a specific binding agent to RANKL, a specific binding agent to TNF, and/or a specific binding agent to IL-1R1 at a concentration of 1 mg/ml to 200 mg/ml and a buffer. In certain embodiments, the buffer is a phosphate buffer or an acetate buffer, at a concentration between 1 mM and 50 mM, and the pH of the formulation is below 6.6. In certain such embodiments, the pharmaceutical formulation comprises a specific binding agent to RANKL at a concentration of 60 mg/ml and acetate buffer at a concentration of 10 mM, and the pH of the formulation is 5.2. In certain embodiments, the specific binding agent to RANKL is an antibody which specifically binds RANKL. In certain embodiments, the antibody is αRANKL-1. In certain embodiments, the pharmaceutical formulation comprises a specific binding agent to TNF at a concentration of 50 mg/ml and phosphate buffer at a concentration of 25 mM, and the pH of the formulation is 6.3. In certain such embodiments, the specific binding agent to TNF is a soluble TNF receptor. In certain embodiments, the soluble TNF receptor is sTNFR:Fc.

In certain embodiments, a pharmaceutical formulation comprises a therapeutically effective amount of a specific binding agent to RANKL, a specific binding agent to TNF, and/or a specific binding agent to IL-1R1; a buffer at a concentration that maintains the pH of the formulation below 6.6; and an amount of an isotonizing agent sufficient to provide a formulation that is isotonic. In certain embodiments, the buffer is a phosphate buffer or an acetate buffer. A formulation that is “isotonic” has an osmolarity between 270 mOsm and 370 mOsm. In certain embodiments, the pH of the formulation is between 4.0 and 6.0. Certain methods of determining the isotonicity of a solution are within the knowledge of those skilled in the art. See, e.g., Setnikar et al., J. Am. Pharm. Assoc. 48:628-30 (1959). Exemplary isotonizing agents include, but are not limited to, sodium chloride; amino acids, including, but not limited to, alanine, arginine, valine, and glycine; sugars and sugar alcohols (polyols), including, but not limited to, glucose, dextrose, fructose, sucrose, maltose, mannitol, trehalose, glycerol, sorbitol, and xylitol; acetic acid, other organic acids or their salts, and relatively minor amounts of citrates or phosphates. In certain embodiments, the isotonizing agent is provided at a concentration of at least 5%. In certain embodiments, the isotonizing agent is sucrose at a concentration of 9%.

In certain embodiments, a pharmaceutical formulation comprises a therapeutically effective amount a specific binding agent to RANKL, a specific binding agent to TNF, and/or a specific binding agent to IL-1R1; a buffer at a concentration that maintains the pH of the formulation below 6.6; and a surfactant. In certain embodiments, the buffer is a phosphate buffer or an acetate buffer. In certain embodiments, the pH of the formulation is between 4.0 and 6.0. In certain embodiments, the surfactant is a nonionic surfactant. Certain exemplary nonionic surfactants include, but are not limited to, polyoxyethylene sorbital esters (polysorbates), polyoxypropylene-polyoxyethylene esters (Pluronic®), polyoxyethylene alcohols, simethicone, polyethylene glycols, lysophosphatidylcholine, and polyoxyethylene-p-t-octylphenols. Certain exemplary surfactants include, but are not limited to, PEG 8000, polysorbate 80 (Tween® 80), and polysorbate 20 (Tween® 20). In certain embodiments, the surfactant is provided at a concentration between 0.001% and 1.0%. In certain embodiments, the surfactant is provided at a concentration between 0.003% and 0.3%. In certain embodiments, the surfactant is provided at a concentration of 0.01%. Those ranges and any ranges discussed in this application include the endpoints and all values between the endpoints.

In certain embodiments, when parenteral administration is contemplated, a therapeutic composition may be in the form of a pyrogen-free, parenterally acceptable aqueous solution comprising a desired specific binding agent to RANKL, desired specific binding agent to TNF, and/or desired specific binding agent to IL-1R1, with or without additional therapeutic agents, in a pharmaceutically acceptable vehicle. In certain embodiments, a vehicle for parenteral injection is sterile distilled water in which a specific binding agent to RANKL, a specific binding agent to TNF, and/or a specific binding agent to IL-1R1, with or without at least one additional therapeutic agent, is formulated as a sterile, isotonic solution, properly preserved. In certain embodiments, the preparation can involve the formulation of the desired molecule with an agent, such as injectable microspheres, bio-erodible particles, polymeric compounds (such as polylactic acid or polyglycolic acid), beads or liposomes, that may provide for the controlled or sustained release of the product which may then be delivered via a depot injection. In certain embodiments, hyaluronic acid may also be used, and may have the effect of promoting sustained duration in the circulation. In certain embodiments, implantable drug delivery devices may be used to introduce the desired molecule.

Additional pharmaceutical compositions will be evident to those skilled in the art, including formulations involving specific binding agents to RANKL, specific binding agents to TNF, and/or specific binding agents to IL-1R1, with or without at least one additional therapeutic agents, in sustained- or controlled-delivery formulations. In certain embodiments, techniques for formulating a variety of other sustained- or controlled-delivery vehicles, such as liposome carriers, bio-erodible microparticles or porous beads and depot injections, are also known to those skilled in the art. See for example, PCT Application No. PCT/US93/00829 which describes the controlled release of porous polymeric microparticles for the delivery of pharmaceutical compositions. In certain embodiments, sustained-release preparations may include semipermeable polymer matrices in the form of shaped articles, e.g. films, or microcapsules. Sustained release matrices may include polyesters, hydrogels, polylactides (U.S. Pat. No. 3,773,919 and EP 058,481), copolymers of L-glutamic acid and gamma ethyl-L-glutamate (Sidman et al., Biopolymers, 22:547-556 (1983)), poly(2-hydroxyethyl-methacrylate) (Langer et al., J. Biomed. Mater. Res., 15:167-277 (1981) and Langer, Chem. Tech., 12:98-105 (1982)), ethylene vinyl acetate (Langer et al., supra) or poly-D(−)-3-hydroxybutyric acid (EP 133,988). In certain embodiments, sustained release compositions may also include liposomes, which can be prepared by any of several methods known in the art. See, e.g., Gabizon et al., Cancer Research 42:4734-4739 (1982); Eppstein et al., Proc. Natl. Acad. Sci. USA, 82:3688-3692 (1985); Szoka et al., Ann. Rev. Biophys. Eng. 9:467-508 (1980); EP 036,676; EP 088,046 and EP 143,949. In certain embodiments, drug delivery systems known in the art are used. Such drug delivery systems are described in, for example, Poznansky et al., Drug Delivery Systems, R. L. Juliano, ed., Oxford, N.Y., pp. 253-315 (1980); Poznansky et al., Pharmacol Rev. 36:277-336 (1984).

The pharmaceutical composition to be used for in vivo administration typically is sterile. In certain embodiments, this may be accomplished by filtration through sterile filtration membranes. In certain embodiments, parenteral compositions generally are placed into a container having a sterile access port, for example, an intravenous solution bag or vial having a stopper pierceable by a hypodermic injection needle. In certain embodiments, parenteral compositions are placed in a syringe suitable for prefilling with the compositions.

In certain embodiments, the effective amount of a pharmaceutical composition comprising a specific binding agent to RANKL, a specific binding agent to TNF, and/or a specific binding agent to IL-1R1, with or without at least one additional therapeutic agent, to be employed therapeutically will depend, for example, upon the therapeutic context and objectives. One skilled in the art will appreciate that the appropriate dosage levels for treatment, according to certain embodiments, will thus vary depending, in part, upon the molecule delivered, the indication for which a specific binding agent to RANKL, a specific binding agent to TNF, and/or a specific binding agent to IL-1R1, with or without at least one additional therapeutic agent, is being used, the route of administration, and the size (body weight, height, body surface and/or organ size) and/or condition (the age, physical condition, and/or general health) of the patient. In certain embodiments, the clinician will consider the severity and history of the disease for which a specific binding agent to RANKL, a specific binding agent to TNF, and/or a specific binding agent to IL-1R1, with or without at least one additional therapeutic agent, is being used. In certain embodiments, the clinician may titer the dosage and modify the route of administration to obtain the optimal therapeutic effect. In certain embodiments, a typical dosage may range from about 0.1 μg/kg to up to about 100 mg/kg or more, depending on the factors mentioned above. In certain embodiments, a higher dosage of specific binding agent to RANKL, specific binding agent to TNF, and/or specific binding agent to IL-1R1 is used with increasing weight of the patient undergoing therapy. In certain embodiments, the dosage may range from 0.1 μg/kg up to about 100 mg/kg; or 1 μg/kg up to about 100 mg/kg; or 5 μg/kg up to about 100 mg/kg.

In certain embodiments, the frequency of dosing will take into account the pharmacokinetic parameters of a specific binding agent to RANKL, a specific binding agent to TNF, and/or a specific binding agent to IL-1R1 and/or any additional therapeutic agents in the formulation used. In certain embodiments, a clinician will administer the composition until a dosage is reached that achieves the desired effect. In certain embodiments, the composition may therefore be administered as a single dose, or as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. In certain embodiments, the effective dosage of a specific binding agent to RANKL, a specific binding agent to TNF, and/or a specific binding agent to IL-1R1 used for treatment increases over the course of a patient treatment. In certain embodiments, the effective dosage of a specific binding agent to RANKL, a specific binding agent to TNF, and/or a specific binding agent to IL-1R1 used for treatment decreases over the course of a patient treatment. In certain embodiments, appropriate dosages may be ascertained through use of appropriate dose-response data.

In certain embodiments, the dosing regimen includes an initial administration of a therapeutically effective dose of a specific binding agent to RANKL, a specific binding agent to TNF, and/or a specific binding agent to IL-1R1, with or without at least one additional therapeutic agent, on days 1, 7, 14, and 21 of a treatment period. In certain embodiments, the dosing regimen includes an initial administration of a therapeutically effective dose of a specific binding agent to RANKL, a specific binding agent to TNF, and/or a specific binding agent to IL-1R1, with or without at least one additional therapeutic agent, on days 1, 2, 3, 4, 5, 6, and 7 of a week in a treatment period. In certain embodiments, the dosing regimen includes an initial administration of a therapeutically effective dose of a specific binding agent to RANKL, a specific binding agent to TNF, and/or a specific binding agent to IL-1R1, with or without at least one additional therapeutic agent, on days 1, 3, 5, and 7 of a week in a treatment period. In certain embodiments, the dosing regimen includes an initial administration of a therapeutically effective dose of a specific binding agent to RANKL, a specific binding agent to TNF, and/or a specific binding agent to IL-1R1, with or without at least one additional therapeutic agent, on days 1 and 3 of a week in a treatment period. In certain embodiments, the dosing regimen includes an initial administration of a therapeutically effective dose of a specific binding agent to RANKL, a specific binding agent to TNF, and/or a specific binding agent to IL-1R1, with or without at least one additional therapeutic agent, on day 1 of a week in a treatment period. In certain embodiments, the treatment period comprises 1 week, 2 weeks, 3 weeks, one month, 3 months, 6 months, one year, or more. In certain embodiments, treatment periods are subsequent or separated from each other by one day, one week, 2 weeks, one month, 3 months, 6 months, one year, or more.

In certain embodiments, the same therapeutically effective dose of a specific binding agent to RANKL, a specific binding agent to TNF, and/or a specific binding agent to IL-1R1 is administered at each dosing over the course of a treatment period. In certain embodiments, different therapeutically effective doses of a specific binding agent to RANKL, a specific binding agent to TNF, and/or a specific binding agent to IL-1R1 are administered at each dosing over the course of a treatment period. In certain embodiments, the same therapeutically effective dose of a specific binding agent to RANKL, a specific binding agent to TNF, and/or a specific binding agent to IL-1R1 is administered at certain dosings over the course of a treatment period and different therapeutically effective doses are administered at certain other dosings.

In certain embodiments, the initial therapeutically effective dose of a specific binding agent to RANKL, a specific binding agent to TNF, and/or a specific binding agent to IL-1R1 is in a lower dosing range, for example, from 0.1 μg/kg up to 20 mg/kg, with subsequent doses in an upper dosing range, for example, from 20 mg/kg up to 100 mg/kg. In certain embodiments, the initial therapeutically effective dose of a specific binding agent to RANKL, a specific binding agent to TNF, and/or a specific binding agent to IL-1R1 is in an upper dosing range, for example, from 20 mg/kg up to 100 mg/kg, with subsequent doses in a lower dosing range, for example, from 0.1 μg/kg up to 20 mg/kg. Those ranges and any ranges discussed in this application include the endpoints and all values between the endpoints.

In certain embodiments, the initial therapeutically effective dose of a specific binding agent to RANKL, a specific binding agent to TNF, and/or a specific binding agent to IL-1R1 is administered as a “loading dose.” “Loading dose” refers to an initial dose of a specific binding agent to RANKL, a specific binding agent to TNF, and/or a specific binding agent to IL-1R1 that is administered to a patient, where the dose of the specific binding agent to RANKL, the specific binding agent to TNF, and/or the specific binding agent to IL-1R1 administered falls within a higher dosing range, for example, 20 mg/kg up to 100 mg/kg. That range and any ranges discussed in this application include the endpoints and all values between the endpoints. In certain embodiments, the loading dose is administered as a single administration, for example, including, but not limited to, a single infusion administered intravenously. In certain embodiments, the loading dose is administered as multiple administrations, for example, including, but not limited to, multiple infusions administered intravenously. In certain embodiments, the loading dose is administered over a 24-hour period. In certain embodiments, after administration of the loading dose, the patient is administered one or more additional therapeutically effective doses of the specific binding agent to RANKL, the specific binding agent to TNF, and/or the specific binding agent to IL-1R1. In certain such embodiments, subsequent therapeutically effective doses of the specific binding agent to RANKL, the specific binding agent to TNF, and/or the specific binding agent to IL-1R1 are administered according to a weekly dosing schedule, for example, but not limited to, once every two weeks, once every three weeks, or once every four weeks. In certain such embodiments, the dose of subsequent therapeutically effective doses falls within a lower dosing range, for example, 0.1 μg/kg up to 20 mg/kg.

In certain embodiments, after administration of the loading dose, the patient is administered one or more additional therapeutically effective doses of the specific binding agent to RANKL, the specific binding agent to TNF, and/or the specific binding agent to IL-1R1 according to a “maintenance schedule.” Exemplary maintenance schedules include, but are not limited to, administration once a month, once every six weeks, once every two months, once every ten weeks, once every three months, once every 14 weeks, once every four months, once every 18 weeks, once every five months, once every 22 weeks, once every six months, once every seven months, once every eight months, once every nine months, once every ten months, once every eleven months, or once every twelve months. In certain embodiments, subsequent doses are administered at more frequent intervals, for example, once every two weeks to once every month. In certain such embodiments, subsequent doses of a specific binding agent to RANKL, a specific binding agent to TNF, and/or a specific binding agent to IL-1R1 fall within a lower dosing range, for example, 0.1 μg/kg up to 20 mg/kg. In certain embodiments, subsequent doses are administered at less frequent intervals, for example, once every month to once every twelve months. In certain such embodiments, subsequent doses of a specific binding agent to RANKL, a specific binding agent to TNF, and/or a specific binding agent to IL-1R1 fall within a higher dosing range, for example, 20 mg/kg up to 100 mg/kg.

In certain embodiments, the route of administration of the pharmaceutical composition is in accord with known methods, e.g. orally, through injection by intravenous, intraperitoneal, intracerebral (intra-parenchymal), intracerebroventricular, intramuscular, intra-ocular, intraarterial, intraportal, or intralesional routes; by sustained release systems or by implantation devices. In certain embodiments, the compositions may be administered by bolus injection or continuously by infusion, or by implantation device.

In certain embodiments, intravenous administration occurs by infusion over a period of 1 to 10 hours. In certain embodiments, intravenous administration occurs by infusion over a period of 1 to 8 hours. In certain embodiments, intravenous administration occurs by infusion over a period of 2 to 7 hours. In certain embodiments, intravenous administration occurs by infusion over a period of 4 to 6 hours. Those ranges and any ranges discussed in this application include the endpoints and all values between the endpoints. In certain embodiments, the infusion period depends on the specific binding agent to RANKL, the specific binding agent to TNF, and/or the specific binding agent to IL-1R1 to be administered. The determination of certain appropriate infusion periods is within the skill of the art. In certain embodiments, the initial infusion is given over a period of 4 to 6 hours, with subsequent infusions delivered more quickly. In certain such embodiments, subsequent infusions are administered over a period of 1 to 6 hours.

In certain embodiments, a specific binding agent to RANKL, a specific binding agent to TNF, and/or a specific binding agent to IL-1R1 and/or any additional therapeutic agents can be placed into syringes and stoppered such that the prefilled syringes have a minimized headspace. In certain embodiments, the specific binding agent to RANKL is an antibody which specifically binds RANKL. In certain embodiments, the antibody is αRANKL-1. In certain embodiments, the specific binding agent to TNF is a soluble TNF receptor. In certain embodiments, the soluble TNF receptor is sTNFR:Fc. In certain embodiments, the specific binding agent to IL-1R1 is an antibody which specifically binds IL-1R1. In certain embodiments, the antibody is selected from 15C4, 26F5 and 27F2 as described in U.S. Publication No. 2004/0097712. In certain embodiments, syringes containing a specific binding agent to RANKL, a specific binding agent to TNF, and/or a specific binding agent to IL-1R1 are stoppered with Fluorotec/B2 coated plungers, for example, including, but not limited to Daikyo/West (Becton Dickinson, part numbers 47165910 and 47165919) and Dupont (Becton Dickinson, part numbers 5080958 and 5115079) using either a vacuum stopper placement method or a mechanical stopper placement method, as described below.

In certain embodiments, a vacuum stopper placement method includes use of a vacuum stopper placement unit, for example, including, but not limited to Autoclavable Stopper Placement Unit (ASPU), ImproSystems Hypak filler, catalog number 897400. In certain embodiments, syringes containing a specific binding agent to RANKL, a specific binding agent to TNF, and/or a specific binding agent to IL-1R1 are placed in the unit and stoppered under 75 pounds per square inch inlet pressure with vacuum cycle settings of FC1—21″ Hg, FC2—6.5″ Hg, FC3 26.5″ Hg. In certain embodiments, those settings result in at least a 3 mm headspace. In certain embodiments, stoppered and prefilled syringes with minimized headspace are produced from stoppered and prefilled syringes having at least a 3 mm headspace by manually manipulating such stoppered and prefilled syringes to express air from the needle by orienting the syringe with the needle up such that the bubble rises to the base of the needle, expelling the air out of the needle, and reshielding of the needle.

In certain embodiments, a mechanical stopper placement method includes use of a mechanical stopper placement unit, for example, including, but not limited to, Groninger, model SVH200. In certain embodiments, syringes containing a specific binding agent to RANKL, a specific binding agent to TNF, and/or a specific binding agent to IL-1R1 are placed in the unit and stoppers are mechanically positioned. In certain embodiments, stoppers are positioned using a vacuum. In certain embodiments, a vent tube is used during the stoppering process. To produce stoppered and prefilled syringes with minimized headspace, the stoppers are positioned against the upper surface of the liquid composition containing a specific binding agent to RANKL, a specific binding agent to TNF, and/or a specific binding agent to IL-1R1 such that the stopper is as close as possible to the liquid surface with a maximum of contact between the bottom surface of the stopper and the upper surface of the liquid. In certain embodiments, the distance between the bottom surface of the stopper and the meniscus is minimized.

In certain embodiments, the headspace of a prefilled and stoppered syringe is measured manually with a calibrated caliper. An exemplary method of calibrating a caliper is to place it in a fully closed position (0.00″) and then calibrate with gauge blocks 0.050″ and 4.000″ according to the manufacturer's instructions. In certain embodiments, the headspace of a prefilled and stoppered syringe is measured with a microscope and microscope ruler. In certain such embodiments, calipers are used to record the distance between the top of the meniscus to the bottom of the flat body of the plunger using calipers. In certain embodiments, the headspace of a prefilled and stoppered syringe is measured with an optical comparator. An exemplary optical comparator is Deltronic DH 216, Horizontal Optical Comparator. In certain such embodiments, measurements are made by placing the syringe in a vertical position and parallel to the optical lens. A magnified image is projected onto a screen for inspection. Calipers on the optical comparator are used to record the distance between the top of the meniscus to the bottom of the flat body of the plunger. In certain embodiments, the headspace is the distance in millimeters from the top of the meniscus to the bottom of the flat body of the plunger.

In certain prefilled syringes, the headspace varies from 2 mm to 5 mm. In certain prefilled syringes, the headspace is 3 mm±0.00254 mm. In certain prefilled syringes having a minimized headspace, the headspace is less then 2.9 mm, or less than 2.7 mm, or less than 2.5 mm, or less than 2.3 mm, or less than 2 mm, or less than 1.5 mm, or less than 1.0 mm, or there is no detectable headspace.

In certain embodiments, syringe barrels comprise material such as, but not limited to, glass, cyclic olefin polymer (“COP”), or cyclic olefin copolymer (“COC”). In certain embodiments, a silicone coating is applied to a syringe barrel. In certain such embodiments, the silicone coating is cross-linked silicone, baked high viscosity silicone, or sprayed-on silicone oil. In certain embodiments, the silicone coating is applied by the syringe manufacturer of the syringe. Certain syringe manufacturers include, but are not limited to, Daikyo, Schott-Form a Vitrum, Bunder, and Becton-Dickinson. In certain embodiments, syringe barrels do not comprise a silicone coating.

In certain embodiments, a syringe plunger is coated. Exemplary syringe plunger coatings include, but are not limited to, polytetrafluoroethylene (PTFE), Teflon®, and ethylene tetrafluoroethylene (ETFE), Fluorotec®. In certain embodiments, the coating is applied by the manufacturer. Certain manufacturers include, but are not limited to, Daikyo and Becton-Dickinson.

### EXAMPLES

### Example 1

The following experiments were performed to evaluate the stability of specific binding agent compositions stored in containers under certain conditions. Stability was monitored under static storage conditions and after shipping. Specifically, certain aspects of syringes were investigated to identify parameters that affect protein aggregation, which, under certain conditions, lead to visible particle formation in the compositions. Various silicone coatings of containers and closures of prefilled syringes were investigated. The specific binding agent used in the experiments below was αRANKL-1.

### General Methods

The concentration of αRANKL-1 in the following experiments varied between 30 mg/ml and 105 mg/ml. αRANKL-1 was formulated in 10 mM sodium acetate, 5% sorbitol, pH 5.2. For experiments using vials, compositions were placed into 3-cc vials to a final volume of 1 ml. For experiments using syringes, 1 ml syringes were used. Compositions in containers were stored for up to 24 months. Compositions in containers were monitored for antibody monomer, high molecular species (aggregates), or low molecular weight species (for example, molecules created by clipping) by native SEC-HPLC or non-reduced, denaturing SEC-HPLC. Visible particles were assessed in compositions in containers by visual inspection of containers as described below.

Native SEC-HPLC was performed using two TSKgel G3000-SWxL 7.8 mm×300 mm columns (Tosoh Bioscience) employed in tandem, with 5 μm particle size and pore size of 250 Å, on an Agilent 1100 Series HPLC with diode array detection. The mobile phase was 100 mM sodium phosphate, 500 mM sodium chloride, 5% ethanol, pH 7.0. The flow rate was 0.5 ml/minute. The sample load was 120 μg protein, and the column eluate was monitored at 235 nm and at 280 nm. Integrated peak areas in the chromatograms were used to quantify the amounts of monomer, which elutes with the main peak; and high molecular weight species, also referred to as aggregates, which elutes with the pre-peak.

Visual inspection of containers for visible particles was conducted in a visual inspection cabinet, the Phoenix Imaging Manual Inspection Booth, catalog no. MIB-100. The visual inspection cabinet has separate, non-reflective black and white surfaces. The black and white surfaces are of sufficient size to serve as a background for the entire container during the inspection process. The visual inspection cabinet also has a light source that provides illumination of at least 2000 Lux at the position of the sample.

To inspect for visible particles, containers were gently swirled or inverted while holding the sample upright at eye level in the visual inspection cabinet. Care was taken to ensure that air bubbles were not introduced while swirling or inverting the containers. Each container was visually observed for approximately five seconds in front of the white surface. Then each container was visually observed for approximately five seconds in front of the black surface. In some cases, a magnifying glass was used, in addition to the light source, to confirm the presence or absence of visible particles.

The presence or absence of visible particles was observed as described above. Then a particle score was assigned to each container and recorded as follows. A score of 0 indicates no particles observed; a score of 1 indicates one or two particles observed; a score of 2 indicates three to nine particles observed; a score of 3 indicates ten to 49 particles observed; a score of 4 indicates 50 or more particles observed.

### Stability in Glass Vials Under Static Storage Conditions

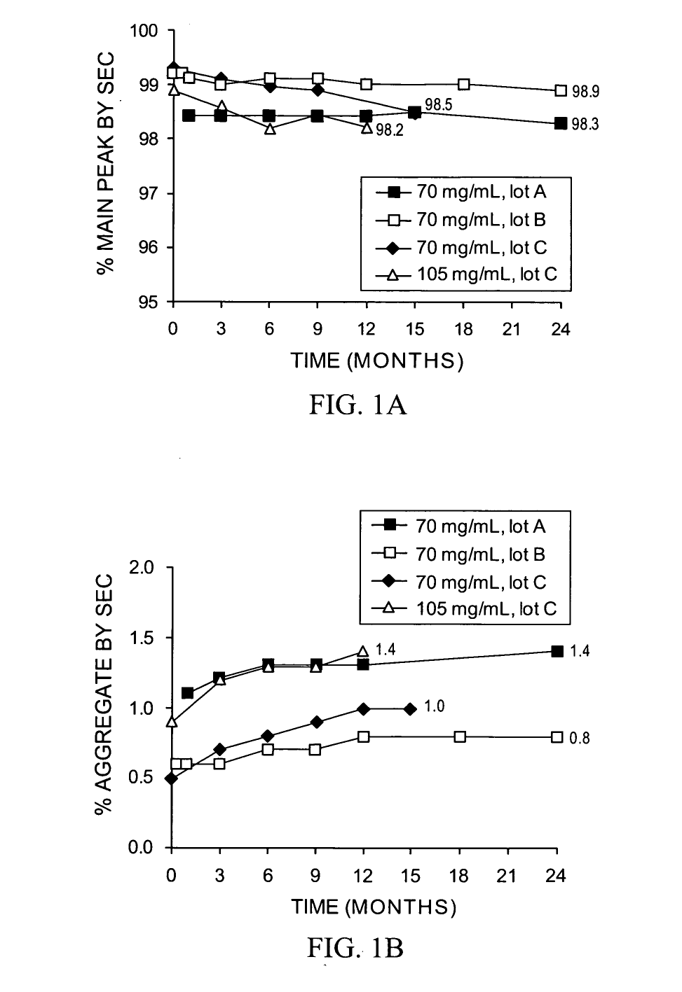

FIG. 1 shows the results of native SEC-HPLC analysis of αRANKL-1 compositions at a protein concentration of either 70 mg/ml or 105 mg/ml, stored in glass vials for 24 months under static conditions, and analyzed at various time points as indicated in the figure. Three different lots were analyzed (lots A, B, and C). FIG. 1 (A) shows the % main peak (monomer) and FIG. 1 (B) shows the aggregate (pre-peak). The results indicate that αRANKL-1 shows little aggregate formation when stored in glass vials at 4° C. for up to 24 months under static conditions. The figure also shows that similar results were obtained for formulations containing 70 mg/ml protein and for formulations containing 105 mg/ml protein.

### Stability in Prefilled Glass Syringes Under Static Storage Conditions

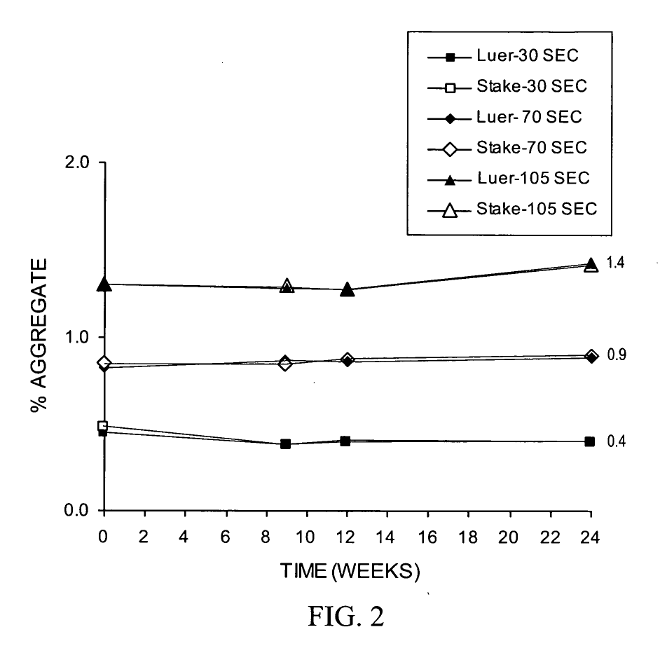

FIG. 2 shows the results of native SEC-HPLC of αRANKL-1 compositions at different protein concentrations, stored in either prefilled glass luer lock syringes or prefilled glass staked-needle syringes, and analyzed at various time points as indicated in the figure. The results indicate that αRANKL-1 shows little aggregate formation when stored under static conditions in either prefilled glass luer lock syringes or prefilled glass staked-needle syringes at 4° C. for up to 24 weeks. The figure also shows that similar results were obtained for formulations containing 30 mg/ml protein, 70 mg/ml protein, and 105 mg/ml protein.

Stability in Prefilled Glass Syringes after Shipping

In contrast to the stability results discussed above, prefilled glass staked needle syringes containing αRANKL-1 at 60 mg/ml protein and shipped by air at a temperature between 2° C. and 8° C., for a distance of 1050 miles, showed visible particles after shipping, as assessed by visual inspection (data not shown).

### Effect of Silicone Coating of Containers and Closures on Shipping Stability

To investigate the effects of various syringe and plunger materials and coatings on particle formation in prefilled syringes after shipping, the following experiment was performed. An αRANKL-1 composition at 60 mg/ml protein was placed into different types of containers having different types of closures, each having different silicone and other coatings as indicated in Tables 1 and 2. The manufacturers and the catalog numbers for the containers and closures used in these experiments are provided in Tables 1 and 2. Containers comprised of glass, cyclic olefin polymer (“COP;” [Resin Cz®]), or cyclic olefin copolymer (“COC”) were tested. Three different silicone coatings were tested: baked-on high viscosity silicone, cross-linked silicone, and sprayed-on silicone oil. Certain containers did not comprise a silicone coating. Two different closure coatings were tested: polytetrafluoroethylene (PTFE), Teflon® and ethylene tetrafluoroethylene (ETFE), Fluorotec®.

Each experimental group listed in Table 3 consisted of 10 containers. The containers were stored at 4° C. for up to one week before being subjected to shipping conditions. The shipping conditions were by air at a temperature ranging from 2° C. to 8° C. within C167 polyurethane shippers according to conditions specified by the American Society for Testing and Materials (ATSM). Prefilled syringes were shipped by air from Thousand Oaks, Calif. to Boulder, Colo., then from Boulder, Colo. to Thousand Oaks, Calif., for a total of two airplane flights (two air pressure cycles; each flight having one air pressure cycle, for take-off and for landing). After shipping, visible particles were assessed in each of the containers by visual inspection. After inspection, a particle score was assigned to each container as described above under General Methods. The results (for all 10 containers in each group) are shown in Table 3.

The results indicate that the particle score was 0 or 1 in the COP syringes, each of which comprised a barrel made of a high molecular weight plastic material lacking silicone. Table 3, group 1. The group 1 syringe closures were coated with PTFE, which lacks silicone. Table 3, group 1. In addition, the particle score was 0 or 1 in COC syringes, each of which comprised a barrel coated with cross-linked silicone. Table 3, group 2. The group 2 syringe closures were coated with Fluorotec B2, which lacks silicone. The particle score was also 0 or 1 in glass syringes, each of which comprised barrels either lacking silicone, or coated with baked-on high viscosity silicone, and having closures coated with either Fluorotec B2 lacking silicone, or Fluorotec B2 and cross-linked silicone. Table 3, groups 3 and 5. In addition, the particle score was 0 or 1 in glass vials lacking silicone, and having closures coated with Fluorotec B2 and cross-linked silicone. Table 3, group 6. In contrast, glass syringes comprising a barrel coated with sprayed-on silicone oil and having closures coated with Fluorotec B2 and cross-linked silicone had a particle score of 4, corresponding to the greatest amount of visible particles. Table 3, group 4. Thus, those results suggest that silicone from prefilled syringe barrels coated with sprayed-on silicone oil contributes to formation of visible particles during shipping.

In addition, two different methods of sterilization were carried out according to standard procedures that are known in the art. Those methods were: E-beam (gamma irradiation) and steam. Two different levels of E-beam sterilization were tested, 15 kGy and 25 kGy. It was found that the neither the sterilization method nor the level of E-beam sterilization affected the particle score (data not shown).

TABLE 1
Containers

| Container Type | | Manufacturer | Catalog Number |
|---|---|---|---|
| 1. | cyclic olefin polymer [Crystal | Daikyo (West, | 2601468 |
| | Zenith (Resin CZ ®)] plastic | distributor) | |
| | syringe lacking silicone | | |
| 2. | cyclic olefin copolymer plastic | Schott-Forma | PG130002 |
| | syringe lacking silicone | Vitrum | |
| 3. | baked-on high viscosity | Bunder | 61650004 |
| | siliconized glass syringe | | |
| 4. | sprayed-on siliconized glass | Becton- | 47217010 |
| | syringe | Dickinson | |
| 5. | glass syringe lacking silicone | Becton- | 47217010 (special |
| | | Dickinson | order without |
| | | | silicone oil) |
| 6. | glass vial lacking silicone | Alcan | 2702B67B |

TABLE 2
Closures

| Closure Type | | Manufacturer | Catalog Number |
|---|---|---|---|
| 1. | PTFE coated stopper | Daikyo (West, | 26014619 |
| | lacking silicone | distributor) | |
| 2. | Flurotec B2 plunger | Daikyo/West | 47205410 (special |
| | (ETFE) lacking silicone | (Becton-Dickinson, | order without |
| | | distributor) | silicone oil) |
| 3. | siliconized Flurotec B2 | Daikyo/West | 47205410 |
| | plunger (ETFE) | (Becton-Dickinson, | |
| | | distributor) | |
| 4. | siliconized Flurotec vial | Daikyo (West, | 19500039 |
| | stopper | distributor) | |

TABLE 3

| | Container | | | Closure | | | |
|---|---|---|---|---|---|---|---|
| | container | | | closure | | | Particle |
| Group | type | material | lubricant | type | coating | lubricant | Score |
| 1 | 1 (syringe) | COP | none | 1 (stopper) | PTFE | none | 0-1 |
| 2 | 2 (syringe) | COC | cross- | 2 (plunger) | Flurotec | none | 0-1 |
| | | | linked | | B2 | | |
| | | | silicone | | | | |
| 3 | 3 (syringe) | glass | baked-on | 2 (plunger) | Flurotec | none | 0-1 |
| | | | high | | B2 | | |
| | | | viscosity | | | | |
| | | | silicone | | | | |
| 4 | 4 (syringe) | glass | sprayed- | 3 (plunger) | Flurotec | cross- | 4 |
| | | | on silicone | | B2 | linked | |
| | | | oil | | | silicone | |
| 5 | 5 (syringe) | glass | none | 3 (plunger) | Flurotec | cross- | 0-1 |
| | | | | | B2 | linked | |
| | | | | | | silicone | |
| 6 | 6 (vial) | glass | none | 4 (stopper) | Flurotec | cross- | 0-1 |
| | | | | | B2 | linked | |
| | | | | | | silicone | |

### Stability in Prefilled Plastic Syringes

In the following experiments, the concentration of αRANKL-1 was either 60 mg/ml or 120 mg/ml. αRANKL-1 was formulated in 10 mM sodium acetate, 5% sorbitol, pH 5.2. αRANKL-1 compositions were sterile filtered by passing the solution through a 0.2 μM cellulose filter. Samples (1.0 ml) were then manually added into 1 ml COP (Resin CZ®) plastic syringes (see Table 1). Syringes with samples in them were stoppered with Fluorotec coated plungers (see Table 2) according to a vacuum stopper placement method as described below.

For the vacuum stopper placement method, a vacuum stopper placement unit (HYPAK® Autoclavable Stopper Placement Unit, ImproSystems, catalog no. 897400) was used. Syringes were placed in the unit and stoppered under 75 pounds per square inch inlet pressure with vacuum cycle settings of FC1—21″ Hg, FC2—6.5″ Hg, FC3 26.5″ Hg. Those settings resulted in a >3 mm headspace, which was not minimized.

In addition, two different methods of sterilization were carried out according to standard procedures that are known in the art. Those methods were: electronic beam (E-Beam) at two different energy levels, 15 kGy or 25 kGy, and steam.

Following aseptically placing of samples in them, and the stoppering procedure, prefilled syringes were stored under static conditions or were subjected to shipping conditions followed by storage under static conditions. The static storage conditions were storage at 4° C. for up to 52 weeks. The shipping conditions were by air at a temperature ranging from 2° C. to 8° C. within C167 polyurethane shippers according to conditions specified by the American Society for Testing and Materials (ATSM). Prefilled syringes were shipped by air from Thousand Oaks, Calif. to Memphis, Tenn., then from Memphis, Tenn. to Puerto Rico, then from Puerto Rico to Memphis, Tenn., and finally from Memphis, Tenn. to Thousand Oaks, Calif., for a total of four airplane flights (four air pressure cycles; each flight having one air pressure cycle, for take-off and for landing). After shipping, the prefilled syringes were stored under static storage conditions at 4° C. for up to 52 weeks.

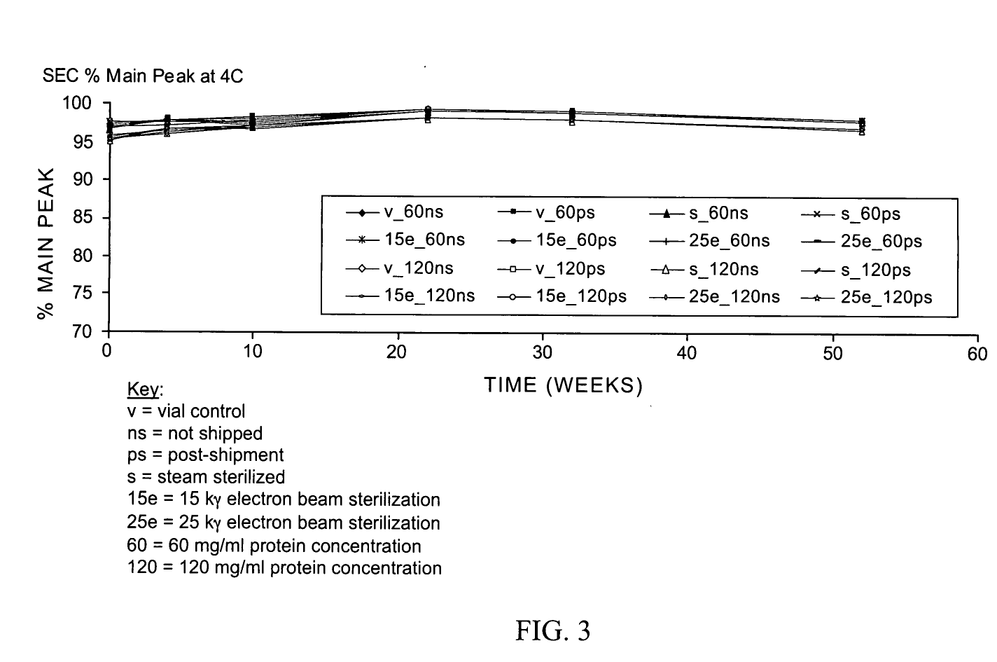

At each timepoint as indicated in FIG. 3, samples were removed from each prefilled syringe for monitoring of antibody monomer, high molecular weight species (aggregates), or low molecular weight species (for example, dimer molecules) by native SEC-HPLC. Native SEC-HPLC was performed using two TSKgel G3000-SWxL 7.8 mm×300 mm columns (Tosoh Bioscience) employed in tandem, with 5 μm particle size and pore size of 250 Å, on an Agilent 1100 Series HPLC with diode array detection. The mobile phase was 100 mM sodium phosphate, 500 mM sodium chloride, 5% ethanol, pH 7.0. The flow rate was 0.5 ml/minute. The sample load was 120 μg protein, and the column eluate was monitored at 235 nm and at 280 nm. Integrated peak areas in the chromatograms were used to quantify the amounts of monomer, which elutes with the main peak; and high molecular weight species, also referred to as aggregates, which elutes with the pre-peak.

FIG. 3 shows the results of the experiments as analyzed by native SEC-HPLC. In FIG. 3, the % main peak (monomer) is shown at each timepoint for each condition tested. The results indicate that αRANKL-1 showed little aggregate formation when placed into COP (Resin CZ®) plastic syringes, stoppered according to a vacuum stopper placement method to form a >3 mm headspace, and stored either under static conditions or subjected to shipping conditions. In addition, two different methods of sterilization were carried out according to standard procedures that are known in the art. Those methods were: E-beam (gamma irradiation) and steam. Two different levels of E-beam sterilization were tested, 15 kGy and 25 kGy. The method of sterilization used in these experiments also did not affect the results.

### Example 2

The results discussed above in Example 1 suggested that plunger movement during shipping of certain prefilled containers contributes to protein aggregation, which may lead to formation of visible particles in the composition. Therefore, parameters that contribute to plunger movement during shipping were considered. One such parameter is headspace. It was hypothesized that the smaller the headspace, the less the amount of plunger movement and consequently, according to the hypothesis, less visible particles would be observed in the composition after shipping. To test that hypothesis, the following experiment was carried out.

The following experiments were performed to assess the effects of minimized headspace on formation of visible particles during shipping of prefilled syringes containing specific binding agent compositions. Different methods of placing compositions in syringes and stoppering syringes to produce minimized headspace were investigated. The specific binding agents used in the experiments below were either sTNFR:Fc or αRANKL-1.

### Visible Particle Analysis

In the following experiments, the concentration of sTNFR:Fc in the compositions was 50 mg/ml. sTNFR:Fc was formulated in 25 mM phosphate, 25 mM arginine HCl, 100 mM NaCl, 1% sucrose, pH 6.3. The concentration of αRANKL-1 was 60 mg/ml. αRANKL-1 was formulated in 10 mM sodium acetate, 5% sorbitol, 0.01% polysorbate-20, pH 5.2.

Specific binding agent compositions were sterile filtered by passing the solution through a 0.2 μM cellulose filter. Samples (1.0 ml) were then manually added into 1 ml Hypak glass syringes (see Table 1). Syringes with samples in them were stoppered with Fluorotec coated plungers (see Table 2) using either a vacuum stopper placement method or a mechanical stopper placement method, as described below.

For the vacuum stopper placement method, a vacuum stopper placement unit (Autoclavable Stopper Placement Unit (ASPU), ImproSystems, catalog number 897400) was used. Syringes containing samples were placed in the unit and stoppered under 75 pounds per square inch inlet pressure with vacuum cycle settings of FC1—24″ Hg, FC2—22.5″ Hg, FC3 26.3″ Hg. The chamber vacuum was 23.5″ Hg. Those settings resulted in a >3 mm headspace. FIG. 6 (A) shows a 4.5 mm headspace. To produce stoppered and prefilled syringes with minimized headspace, syringes containing samples were placed in the unit and stoppered under 75 pounds per square inch inlet pressure with vacuum cycle settings of FC1—24″ Hg, FC2—22.5″ Hg, FC3 29.2″ Hg. The chamber vacuum was 27.5″ Hg. Those settings resulted in a minimized headspace. FIG. 6 (B) shows 1.5 mm headspaces, one with a meniscus (left side of FIG. 6 (B)) and one with an air bubble (right side of FIG. 6(B)).

To manually produce stoppered and prefilled syringes with minimized headspace, the stoppered and prefilled syringes from the unit were manually manipulated to express air from the needle by orienting the syringe with the needle up such that the bubble rises to the base of the needle, expelling the air out of the needle, and reshielding of the needle. As a control for that procedure, a control group of stoppered and prefilled syringes were manually manipulated to express air from the needle, then the plunger was pulled back to approximate the original stopper position and form a >3 mm headspace followed by reshielding of the needle.

For the mechanical stopper placement method, a mechanical stopper placement unit (Groninger, model SVH200) was used. Syringes containing samples were placed in the unit and stoppers were mechanically positioned. To perform this method, a stopper placement tube of smaller diameter than the syringe placed the stopper within the syringe barrel. The stopper placement tube was then retracted, and the stopper expanded to fill the syringe barrel. To produce stoppered and prefilled syringes with minimized headspace, the stoppers were positioned against the upper surface of the liquid composition such that the stopper was as close as possible to the liquid surface with a maximum of contact between the bottom surface of the stopper and the upper surface of the liquid.

The headspace for each prefilled and stoppered syringe was measured manually with a calibrated caliper. The caliper was calibrated by placing it in a fully closed position (0.00″) and then calibrating with gauge blocks 0.050″ and 4.000″according to the manufacturer's instructions. The headspace is the distance in millimeters from the top of the meniscus to the bottom of the flat body of the plunger. In certain prefilled syringes, the headspace varied from 2 mm to 5 mm. In certain prefilled syringes, the headspace was 3 mm±0.001 0.00254 mm. In certain prefilled syringes having a minimized headspace, the headspace was less than 2 mm. In certain prefilled syringes having a minimized headspace, the headspace was less than 1.3 mm.

Prefilled syringes were packaged in boxes and shipped by air at a temperature ranging from 2° C. to 8° C. within C167 polyurethane shippers according to conditions specified by the American Society for Testing and Materials (ATSM). Prefilled syringes were shipped by air from Thousand Oaks, Calif. to Memphis, Tenn., then from Memphis, Tenn. to Puerto Rico, then from Puerto Rico to Memphis, Tenn., and finally from Memphis, Tenn. to Thousand Oaks, Calif., for a total of four airplane flights (four air pressure cycles; each flight having one air pressure cycle, for take-off and for landing). The total transit time was four days or less.

Visual inspection of containers for visible particles was conducted in a visual inspection cabinet the Phoenix Imaging Manual Inspection Booth, catalog no. MIB-100. The visual inspection cabinet has two separate surfaces that are each used as a background for visual inspection of a container. One surface is a non-reflective white surface and the second surface is a non-reflective black surface. The white and black surfaces are of sufficient size so that they may be used as a background for the entire container during the inspection process. The visual inspection cabinet has a light source that provides illumination of at least 2000 Lux at the position of the sample.

To inspect for visible particles, containers were gently swirled or inverted while holding the sample upright at eye level in the visual inspection cabinet. Care was taken to ensure that air bubbles were not introduced while swirling or inverting the containers. Each container was visually observed for approximately five seconds in front of the white surface. Then each container was visually observed for approximately five seconds in front of the black surface. In some cases, a magnifying glass was used, in addition to the light source, to confirm the presence or absence of visible particles.

The presence or absence of visible particles was observed as described above. Then a particle score was assigned to each container and recorded as follows. A score of 0 indicates no particles observed; a score of 1 indicates one or two particles observed; a score of 2 indicates three to nine particles observed; a score of 3 indicates ten to 49 particles observed; a score of 4 indicates 50 or more particles observed.

The results of experiments with prefilled syringes containing αRANKL-1 compositions are shown in Table 4 below. The results show that none of the syringes containing an αRANKL-1 composition and stoppered according to the vacuum stopper placement method to form either a >3 mm headspace or a minimized headspace had visible particles after shipping. The results also show that none of the syringes containing an αRANKL-1 composition and stoppered according to the mechanical stopper placement method to form a minimized headspace had visible particles after shipping.

TABLE 4
Prefilled syringes containing αRANKL-1.

| | | Total | |
|---|---|---|---|
| | | Number of | Number with |
| Stopper Placement | | prefilled | visible |
| Method | Headspace | syringes | particles |
| Vacuum | >3 mm | 100 | 0 |
| Vacuum | Minimized | 20 | 0 |
| | (<1 mm) | | |
| Vacuum | >3 mm | 20 | 0 |
| (control for minimized | | | |
| headspace method) | | | |
| Mechanical | Minimized | 35 | 0 |
| | (<1 mm) | | |

The results of experiments with prefilled syringes containing sTNFR:Fc are shown in Table 5. The results show that all syringes containing a sTNFR:Fc composition and stoppered according to the vacuum stopper placement method to form a >3 mm headspace had visible particles after shipping. 29 (of 30 total) prefilled syringes had a particle score of 3 and one prefilled syringe had a particle score of 2. The results also show that syringes stoppered according to the vacuum stopper placement method to form a minimized headspace reduced the number of visible particles observed after shipping. In that experiment, 29 prefilled syringes had a particle score of 0, while two prefilled syringes had a particle score of 2. The two prefilled syringes that had a particle score of 2 also had a small air bubble remaining after the air was expressed suggesting that the headspace for those syringes was not minimized. In addition, ten prefilled control syringes were tested. The prefilled control syringes were first stoppered according to the vacuum stopper placement method to form a minimized headspace. That method was then followed by repositioning of the plunger to form a >3 mm headspace. As shown in Table 5, all ten prefilled control syringes had visible particles after shipping and each had a particle score of 3.

The results in Table 5 also show that all syringes containing a sTNFR:Fc composition and stoppered according to the mechanical stopper placement method to form a >3 mm headspace had visible particles after shipping. Six (of 10 total) prefilled syringes had a particle score of 3 and four prefilled syringes had a particle score of 2. In addition, the results in Table 5 show that syringes stoppered according to the mechanical stopper placement method to form a minimized headspace reduced the number of visible particles observed after shipping. In that experiment, all 30 prefilled syringes had a particle score of 0.

In summary, the results of stoppering syringes according to two different methods to form a minimized headspace suggested that manufacturing syringes containing specific binding agent compositions and stoppering them according to a method to form a minimized headspace is desirable to reduce or eliminate formation of visible particles during shipping.

TABLE 5
Prefilled syringes containing sTNFR:Fc.

| | | Total | | | | |
|---|---|---|---|---|---|---|
| | | No. of | | | | |
| Stopper Placement | | prefilled | Particle Score | | | |
| Method | Headspace | syringes | 0 | 1 | 2 | 3 |
| Vacuum | >3 mm | 30 | 0 | 0 | 1 | 29 |
| Vacuum | Minimized | 31 | 29 | 0 | 2* | 0 |
| | (<1 mm) | | | | | |
| Vacuum | >3 mm | 10 | 0 | 0 | 0 | 10 |
| (control for minimized | | | | | | |
| headspace method) | | | | | | |
| Mechanical | >3 mm | 10 | 0 | 0 | 4 | 6 |
| Mechanical | Minimized | 30 | 30 | 0 | 0 | 0 |
| | (<1 mm) | | | | | |

*There was a small air bubble in each of these two prefilled syringes.

### Sub-Visible Particle Analysis

In addition to visual inspection of containers for visible particles, sub-visible particle analysis using a Malvern Zetasizer instrument (Malvern, Zetasizer Nano ZS, model no. ZEN3600) was performed on prefilled syringes containing sTNFR:Fc compositions under various conditions.

In the following experiments, the concentration of sTNFR:Fc in the compositions was 50 mg/ml. sTNFR:Fc was formulated in 25 mM phosphate, 25 mM arginine HCl, 100 mM NaCl, 1% sucrose, pH 6.3.

sTNFR:Fc compositions were sterile filtered by passing the solution through a 0.2 μM cellulose filter. Samples (1.0 ml) were then manually added into 1 ml Hypak glass syringes (see Table 1). Syringes containing samples were stoppered with Fluorotec coated plungers (see Table 2) using either a vacuum stopper placement method or a mechanical stopper placement method, as described below.

For the vacuum stopper placement method, a vacuum stopper placement unit (Autoclavable Stopper Placement Unit (ASPU), ImproSystems, catalog number 897400) was used. Syringes containing samples were placed in the unit and stoppered under 75 pounds per square inch inlet pressure with vacuum cycle settings of FC1—24″ Hg, FC2—22.5″ Hg, FC3 26.3″ Hg. The chamber vacuum was 23.5″ Hg. Those settings resulted in a >3 mm headspace.

For the mechanical stopper placement method, a mechanical stopper placement unit (Groninger, model SVH200) was used. Syringes containing samples were placed in the unit and stoppers were mechanically positioned. To perform this method, a stopper placement tube of smaller diameter than the syringe placed the stopper within the syringe barrel. The stopper placement tube was then retracted, and the stopper expanded to fill the syringe barrel. To produce stoppered and prefilled syringes with minimized headspace, the stoppers were positioned against the upper surface of the liquid composition such that the stopper was as close as possible to the liquid surface with a maximum of contact between the bottom surface of the stopper and the upper surface of the liquid.

The headspace for each prefilled and stoppered syringe was measured manually with a calibrated caliper as described in the section entitled “Visible Particle Analysis” above.

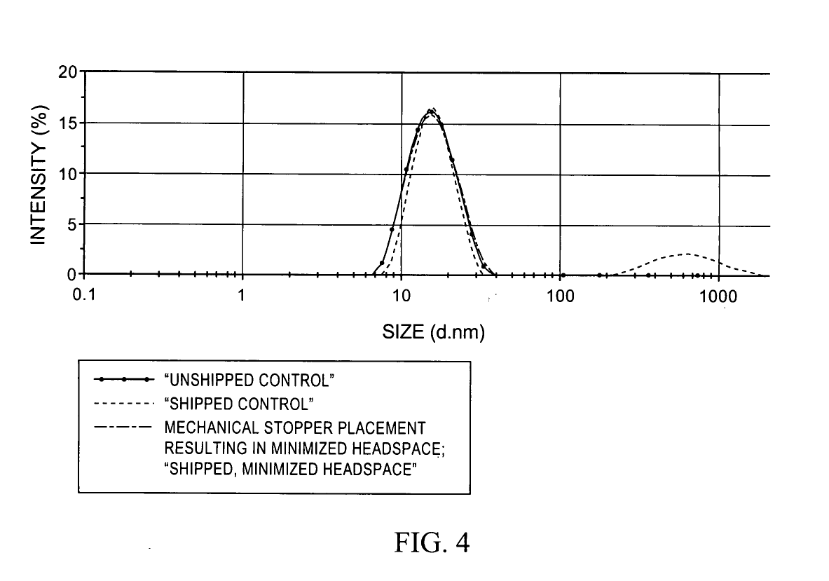

Three prefilled syringes were tested, each under different conditions. A sTNFR:Fc composition was added to one syringe and the syringe was stoppered according to the vacuum stopper placement method to form a >3 mm headspace and was stored under static conditions (FIG. 4, green line, designated “unshipped control”). A sTNFR:Fc composition was also added to a second syringe and the syringe was stoppered according to the vacuum stopper placement method to form a >3 mm headspace and was subjected to shipping conditions as described below (FIG. 4, blue line, designated “shipped control”). A sTNFR:Fc composition was added to the third syringe and the syringe was stoppered according to the mechanical stopper placement method, followed by the procedure described above under the subtitle “visible particle analysis,” to form a minimized headspace (FIG. 4, red line, designated “shipped, minimized headspace”), and was subjected to shipping conditions as follows. For shipping, the prefilled syringes were packaged in boxes and shipped by air at a temperature ranging from 2° C. to 8° C. within C167 polyurethane shippers according to conditions specified by the American Society for Testing and Materials (ATSM). Prefilled syringes were shipped by air from Thousand Oaks, Calif. to Memphis, Tenn., then from Memphis, Tenn. to Puerto Rico, then from Puerto Rico to Memphis, Tenn., and finally from Memphis, Tenn. to Thousand Oaks, Calif., for a total of four airplane flights (four air pressure cycles; each flight having one air pressure cycle, for take-off and for landing). The total transit time was four days or less.

To measure sub-visible particle size using the Malvern Zetasizer instrument, 1 ml sample volumes were placed in a disposable cuvette and measurements were performed at 25° C. Each 1 ml sample was analyzed by five sub-runs of 10 seconds each. A sub-run is a replicate measurement of each sample. Hydrodynamic diameter and polydispersity values were calculated using Dispersants Manager software, using a dispersant viscosity of 0.939 cP.

The intensity weighted size distribution is shown in FIG. 4. Intensity-weighted size distribution is the signal based on the intensity of the light scattered. The results show that the sample from the prefilled syringe designated “shipped control” in FIG. 4 (blue line), had a bimodal distribution with a distinct new peak of large hydrodynamic size. The results also show that the sample from the prefilled syringe designated “unshipped control” in FIG. 4 (green line), and the sample from the prefilled syringe designated “shipped, minimized headspace” in FIG. 4 (red line), did not have the distinct new peak of large hydrodynamic size.

The numerical results of the same experiment are presented in Table 6. Those results show that the sample from the prefilled syringe designated “shipped control” had a larger z-average hydrodynamic diameter and greater polydispersity compared to the sample from the prefilled syringe designated “unshipped control” and the prefilled syringe designated “shipped, minimized headspace”.

TABLE 6

| | Z-average | |
|---|---|---|
| | hydrodynamic | |
| Sample | diameter (nm) | Polydispersity index |
| “unshipped control” | 14.5 | 0.206 |
| “shipped control” | 17.4 | 0.360 |
| “shipped, minimized headspace” | 14.7 | 0.210 |

The results of these experiments suggested that sub-visible particles were not present in the prefilled syringe after shipping, when the syringe was stoppered according to the mechanical stopper placement method to form a minimized headspace. The results thus suggested that manufacturing syringes containing specific binding agent compositions and stoppering them according to a method to form a minimized headspace is desirable to reduce or eliminate formation of particles, including visible and sub-visible particles, during shipping.

We claim:

1. A prefilled syringe containing a composition comprising a specific binding agent, wherein the specific binding agent contained in the prefilled syringe is stabilized.

2. The prefilled syringe of claim 1, wherein the specific binding agent is selected from a specific binding agent to RANKL, a specific binding agent to TNF, and a specific binding agent to IL-1R1.

3. The prefilled syringe of claim 2, wherein the specific binding agent is selected from an antibody, a polyclonal antibody, a monoclonal antibody, an antibody wherein the heavy chain and the light chain are connected by a flexible linker, an Fv molecule, a maxibody, an antigen binding fragment, a Fab fragment, a Fab′ fragment, a F(ab′)₂ molecule, a fully human antibody, a humanized antibody, and a chimeric antibody.

4. The prefilled syringe of claim 3, wherein the specific binding agent is an antibody selected from an antibody that substantially inhibits binding of RANKL to RANK, an antibody that substantially inhibits binding of TNF to TNF-R, and an antibody that substantially inhibits binding of IL-1 to IL-1R1.

5. The prefilled syringe of claim 4, wherein the antibody is an antibody that substantially inhibits binding of RANKL to RANK, wherein the antibody is αRANKL-1, wherein αRANKL-1 comprises a heavy chain and a light chain, wherein the heavy chain comprises an amino acid sequence as set forth in SEQ ID NO: 2 or a fragment thereof, and the light chain comprises an amino acid sequence as set forth in SEQ ID NO: 4 or a fragment thereof.

6. The prefilled syringe of claim 4, wherein the antibody is an antibody that substantially inhibits binding of IL-1 to IL-1R1.

7. The prefilled syringe of claim 1, wherein the composition further comprises at least one additional pharmaceutical agent.

8. The prefilled syringe of claim 1, wherein the composition further comprises at least one stabilizing agent and a buffering agent.

9. The prefilled syringe of claim 8, wherein at least one stabilizing agent is a surfactant.

10. The prefilled syringe of claim 9, wherein the surfactant is selected from polysorbate and polyoxypropylene-polyoxyethylene esters (Pluronic®).

11. The prefilled syringe of claim 10, wherein the surfactant is polysorbate.

12. The prefilled syringe of claim 11, wherein the polysorbate is selected from polysorbate 20 and polysorbate 80.

13. The prefilled syringe of claim 9, wherein the surfactant is present at a concentration of 0.001% to 1%.

14. The prefilled syringe of claim 13, wherein the surfactant is present at a concentration of 0.002% to 0.5%.

15. The prefilled syringe of claim 14, wherein the surfactant is present at a concentration of 0.004%.

16. The prefilled syringe of claim 14, wherein the surfactant is present at a concentration of 0.01%.

17. The prefilled syringe of claim 8, wherein the pH of the composition is below 6.6.

18. The prefilled syringe of claim 17, wherein the pH of the composition is between 5.5 and 6.5.

19. The prefilled syringe of claim 18, wherein the pH of the composition is 6.3.

20. The prefilled syringe of claim 17, wherein the pH of the composition is between 4.5 and 5.5.

21. The prefilled syringe of claim 20, wherein the pH of the composition is 5.2.

22. The prefilled syringe of claim 1, wherein the syringe comprises a material comprising silicone, wherein the silicone is cross-linked.

23. The prefilled syringe of claim 1, wherein the syringe comprises a material comprising silicone, wherein the silicone is baked.

24. The prefilled syringe of claim 1, wherein the syringe lacks silicone, and wherein the syringe closure lacks silicone.

25. The prefilled syringe of claim 1, wherein the syringe comprises a high molecular weight plastic material, wherein the high molecular weight plastic material lacks silicone.

26. The prefilled syringe of claim 25, wherein the high molecular weight plastic material comprises cyclic olefin polymer.

27. The prefilled syringe of claim 25, wherein the high molecular weight plastic material comprises cyclic olefin copolymer.

28. A prefilled syringe containing a composition comprising a specific binding agent, wherein a headspace between the composition and a syringe closure is minimized, and wherein the specific binding agent contained in the prefilled syringe is stabilized.

29. The prefilled syringe of claim 28, wherein the minimized headspace is between 2.5 mm and 3.0 mm.

30. The prefilled syringe of claim 28, wherein the minimized headspace is between 2.0 mm and 2.5 mm.

31. The prefilled syringe of claim 28, wherein the minimized headspace is between 1.5 mm and 2.0 mm.

32. The prefilled syringe of claim 28, wherein the minimized headspace is between 1.0 mm and 1.5 mm.

33. The prefilled syringe of claim 28, wherein the specific binding agent is selected from a specific binding agent to RANKL, a specific binding agent to TNF, and a specific binding agent to IL-1R1.

34. The prefilled syringe of claim 33, wherein the specific binding agent is selected from an antibody, a polyclonal antibody, a monoclonal antibody, an antibody wherein the heavy chain and the light chain are connected by a flexible linker, an Fv molecule, a maxibody, an antigen binding fragment, a Fab fragment, a Fab′ fragment, a F(ab′)₂ molecule, a fully human antibody, a humanized antibody, and a chimeric antibody.

35. The prefilled syringe of claim 34, wherein the specific binding agent is an antibody selected from an antibody that substantially inhibits binding of RANKL to RANK, an antibody that substantially inhibits binding of TNF to TNF-R, and an antibody that substantially inhibits binding of IL-1 to IL-1R1.

36. The prefilled syringe of claim 35, wherein the antibody is an antibody that substantially inhibits binding of RANKL to RANK, wherein the antibody is αRANKL-1, wherein αRANKL-1 comprises a heavy chain and a light chain, wherein the heavy chain comprises an amino acid sequence as set forth in SEQ ID NO: 2 or a fragment thereof, and the light chain comprises an amino acid sequence as set forth in SEQ ID NO: 4 or a fragment thereof.

37. The prefilled syringe of claim 35, wherein the antibody is an antibody that substantially inhibits binding of IL-1 to IL-1R1.

38. The prefilled syringe of claim 28, wherein the composition further comprises at least one additional pharmaceutical agent.

39. The prefilled syringe of claim 28, wherein the composition further comprises at least one stabilizing agent and a buffering agent.

40. The prefilled syringe of claim 39, wherein at least one stabilizing agent is a surfactant.

41. The prefilled syringe of claim 40, wherein the surfactant is selected from polysorbate and polyoxypropylene-polyoxyethylene esters (Pluronic®).

42. The prefilled syringe of claim 41, wherein the surfactant is polysorbate.

43. The prefilled syringe of claim 42, wherein the polysorbate is selected from polysorbate 20 and polysorbate 80.

44. The prefilled syringe of claim 40, wherein the surfactant is present at a concentration of 0.001% to 1%.

45. The prefilled syringe of claim 44, wherein the surfactant is present at a concentration of 0.002% to 0.5%.

46. The prefilled syringe of claim 45, wherein the surfactant is present at a concentration of 0.004%.

47. The prefilled syringe of claim 45, wherein the surfactant is present at a concentration of 0.01%.

48. The prefilled syringe of claim 39, wherein the pH of the composition is below 6.6.

49. The prefilled syringe of claim 48, wherein the pH of the composition is between 5.5 and 6.5.

50. The prefilled syringe of claim 49, wherein the pH of the composition is 6.3.

51. The prefilled syringe of claim 48, wherein the pH of the composition is between 4.5 and 5.5.

52. The prefilled syringe of claim 51, wherein the pH of the composition is 5.2.

53. The prefilled syringe of claim 28, wherein the syringe comprises a material comprising silicone, wherein the silicone is cross-linked.

54. The prefilled syringe of claim 28, wherein the syringe comprises a material comprising silicone, wherein the silicone is baked.

55. The prefilled syringe of claim 28, wherein the syringe lacks silicone, and wherein the syringe closure lacks silicone.

56. The prefilled syringe of claim 28, wherein the syringe comprises a high molecular weight plastic material, wherein the high molecular weight plastic material lacks silicone.

57. The prefilled syringe of claim 56, wherein the high molecular weight plastic material comprises cyclic olefin polymer.

58. The prefilled syringe of claim 56, wherein the high molecular weight plastic material comprises cyclic olefin copolymer.

59. A method of preparing a prefilled syringe comprising introducing into the syringe a composition comprising a specific binding agent such that a headspace between the composition and a syringe closure is minimized, and wherein the specific binding agent contained in the prefilled syringe is stabilized.

60. The method of claim 59, wherein the specific binding agent is selected from a specific binding agent to RANKL, a specific binding agent to TNF, and a specific binding agent to IL-1R1.

61. The method of claim 60, wherein the specific binding agent is selected from an antibody, a polyclonal antibody, a monoclonal antibody, an antibody wherein the heavy chain and the light chain are connected by a flexible linker, an Fv molecule, a maxibody, an antigen binding fragment, a Fab fragment, a Fab′ fragment, a F(ab′)₂ molecule, a fully human antibody, a humanized antibody, and a chimeric antibody.

62. The method of claim 61, wherein the specific binding agent is an antibody selected from an antibody that substantially inhibits binding of RANKL to RANK, an antibody that substantially inhibits binding of TNF to TNF-R, and an antibody that substantially inhibits binding of IL-1 to IL-1R1.

63. The method of claim 62, wherein the antibody is an antibody that substantially inhibits binding of RANKL to RANK, wherein the antibody is αRANKL-1, wherein αRANKL-1 comprises a heavy chain and a light chain, wherein the heavy chain comprises an amino acid sequence as set forth in SEQ ID NO: 2 or a fragment thereof, and the light chain comprises an amino acid sequence as set forth in SEQ ID NO: 4 or a fragment thereof.

64. The method of claim 62, wherein the antibody is an antibody that substantially inhibits binding of IL-1 to IL-1R1.

65. The method of claim 59, wherein the composition further comprises at least one additional pharmaceutical agent.

66. The method of claim 59, wherein the composition further comprises at least one stabilizing agent and a buffering agent.

67. The method of claim 66, wherein at least one stabilizing agent is a surfactant.

68. The method of claim 67, wherein the surfactant is selected from polysorbate and polyoxypropylene-polyoxyethylene esters (Pluronic®).

69. The method of claim 68, wherein the surfactant is polysorbate.

70. The method of claim 69, wherein the polysorbate is selected from polysorbate 20 and polysorbate 80.

71. The method of claim 67, wherein the surfactant is present at a concentration of 0.001% to 1%.

72. The method of claim 71, wherein the surfactant is present at a concentration of 0.002% to 0.5%.

73. The method of claim 72, wherein the surfactant is present at a concentration of 0.004%.

74. The method of claim 72, wherein the surfactant is present at a concentration of 0.01%.

75. The method of claim 66, wherein the pH of the composition is below 6.6.

76. The method of claim 75, wherein the pH of the composition is between 5.5 and 6.5.

77. The method of claim 76, wherein the pH of the composition is 6.3.

78. The method of claim 75, wherein the pH of the composition is between 4.5 and 5.5.

79. The method of claim 78, wherein the pH of the composition is 5.2.

80. The method of claim 59, wherein the syringe comprises a material comprising silicone, wherein the silicone is cross-linked.

81. The method of claim 59, wherein the syringe comprises a material comprising silicone, wherein the silicone is baked.

82. The method of claim 59, wherein the syringe lacks silicone, and wherein the syringe closure lacks silicone.

83. The method of claim 59, wherein the syringe comprises a high molecular weight plastic material, wherein the high molecular weight plastic material lacks silicone.

84. The method of claim 83, wherein the high molecular weight plastic material comprises cyclic olefin polymer.

85. The method of claim 83, wherein the high molecular weight plastic material comprises cyclic olefin copolymer.

86. The prefilled syringe of claim 1 or claim 28, wherein the specific binding agent is at a concentration of 1 mg/ml to 150 mg/ml.

87. The method of claim 59, wherein the specific binding agent is at a concentration of 1 mg/ml to 150 mg/ml.

88. A method for stabilizing a specific binding agent in a composition, wherein the composition is contained in a prefilled syringe, comprising placing the composition in the prefilled syringe such that a headspace between the composition and a syringe closure is minimized, and wherein the specific binding agent contained in the prefilled syringe is stabilized.
